SEQUENCE LISTING (68 sequences)

SEQ ID NO: 1 - protein, 5 aa, synthetic construct
SYSMN

SEQ ID NO: 2 - protein, 17 aa, synthetic construct
SISSSSSYIYYADSVKG

SEQ ID NO: 3 - protein, 12 aa, synthetic construct
RHGYSNSDAFDN

SEQ ID NO: 4 - protein, 11 aa, synthetic construct
RASQGISHYLV

SEQ ID NO: 5 - protein, 7 aa, synthetic construct
AASSLQS

SEQ ID NO: 6 - protein, 9 aa, synthetic construct
LQHNSYPWT

SEQ ID NO: 7 - protein, 121 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSS

SEQ ID NO: 8 - protein, 107 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIK

SEQ ID NO: 9 - protein, 223 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKC

SEQ ID NO: 10 - protein, 214 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 381 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKCDKTHTGGGGQGGGGQGGGGQGGGGQGGGGQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 12 - protein, 214 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQCGNSQESVTEQDSKDSTYSLSSTLTLSKADYEKHKVYACEVTHQGLSSPVTKSFNRGEC

SEQ ID NO: 13 - protein, 447 aa, synthetic construct
  VARIANT 128: note = X is S or C
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPXVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 14 - protein, 447 aa, synthetic construct
  VARIANT 379: note = X is A or C
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVSTLPPSQEEMTKNQVSLMCLVYGFYPSDIXVEWESNGQPENNYKTTPPVLDSDGSFFLYSVLTVDKSRWQEGNVFSCSVMHEALHNHYTQKSLSLSLG

SEQ ID NO: 15 - protein, 228 aa, synthetic construct
  VARIANT 160: note = X is A or C
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQGDMTKNQVQLTCLVKGFYPSDIXVEWESNGQPENNYKTTPPVLDSDGSFFLASRLTVDKSRWQEGNVFSCSVMHEALHNHYTQKSLSLSLG

SEQ ID NO: 16 - protein, 447 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVSTLPPSQEEMTKNQVSLMCLVYGFYPSDIAVEWESNGQPENNYKTTPPVLDSDGSFFLYSVLTVDKSRWQEGNVFSCSVMHEALHNHYTQKSLSLSLG

SEQ ID NO: 17 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQGDMTKNQVQLTCLVKGFYPSDIAVEWESNGQPENNYKTTPPVLDSDGSFFLASRLTVDKSRWQEGNVFSCSVMHEALHNHYTQKSLSLSLG

SEQ ID NO: 18 - protein, 446 aa, synthetic construct
  VARIANT 127: note = X is S or C
QVQLVQSGAEVKKPGSSVKVSCKASGYTFSSYAIEWVRQAPGQGLEWMGGILPGSGTINYNEKFKGRVTITADKSTSTAYMELSSLRSEDTAVYYCARMSSNSDQGFDLWGQGTLVTVSSASTKGPXVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLG

SEQ ID NO: 19 - protein, 214 aa, synthetic construct
DIQMTQSPSSLSASVGDRVTITCKASQGISRFLSWFQQKPGKAPKSLIYAVSSLVDGVPSRFSGSGSGTDFTLTISSLQPEDFATYYCVQYNSYPYGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 5 aa, synthetic construct
ETAVA

SEQ ID NO: 21 - protein, 17 aa, synthetic construct
GIGGGVDITYYADSVKG

SEQ ID NO: 22 - protein, 17 aa, synthetic construct
RPGRPLITSKVADLYPY

SEQ ID NO: 23 - protein, 128 aa, unidentified
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 24 - protein, 20 aa, synthetic construct
GGGGQGGGGQGGGGQGGGGQ

SEQ ID NO: 25 - protein, 6 aa, synthetic construct
GSYWIC

SEQ ID NO: 26 - protein, 18 aa, synthetic construct
CIYSTSGGRTYYASWVKG

SEQ ID NO: 27 - protein, 12 aa, synthetic construct
GDDSISDAYFDL

SEQ ID NO: 28 - protein, 13 aa, synthetic construct
QSSQSVYNNNRLA

SEQ ID NO: 29 - protein, 7 aa, synthetic construct
DASTLAS

SEQ ID NO: 30 - protein, 12 aa, synthetic construct
QGTYFSSGWSWA

SEQ ID NO: 31 - protein, 121 aa, synthetic construct
QSLEESGGDLVKPEGSLTLTCTASGFSFSGSYWICWVRQAPGKGLEWIGCIYSTSGGRTYYASWVKGRFTISKTSSTTVTLQMTSLTAADTATYFCARGDDSISDAYFDLWGPGTLVTVSS

SEQ ID NO: 32 - protein, 112 aa, synthetic construct
ALDMTQTASPVSAAVGGTVTINCQSSQSVYNNNRLAWYQQKPGQPPKLLIYDASTLASGVPSRFKGSGSGTQFTLTISGVQSDDSATYYCQGTYFSSGWSWAFGGGTEVVVK

SEQ ID NO: 33 - protein, 447 aa, synthetic construct
QSLEESGGDLVKPEGSLTLTCTASGFSFSGSYWICWVRQAPGKGLEWIGCIYSTSGGRTYYASWVKGRFTISKTSSTTVTLQMTSLTAADTATYFCARGDDSISDAYFDLWGPGTLVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 34 - protein, 219 aa, synthetic construct
ALDMTQTASPVSAAVGGTVTINCQSSQSVYNNNRLAWYQQKPGQPPKLLIYDASTLASGVPSRFKGSGSGTQFTLTISGVQSDDSATYYCQGTYFSSGWSWAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - RNA, 21 nt, synthetic construct
  source 1..21: mol_type = other RNA
gttgctcgactctaaccgcta

SEQ ID NO: 36 - RNA, 23 nt, synthetic construct
  source 1..23: mol_type = other RNA
tagcggttagagtcgagcaacgg

SEQ ID NO: 37 - RNA, 21 nt, synthetic construct
  source 1..21: mol_type = other RNA
ttcgccaactattctacgtga

SEQ ID NO: 38 - RNA, 23 nt, synthetic construct
  source 1..23: mol_type = other RNA
tcacgtagaatagttggcgaagg

SEQ ID NO: 39 - RNA, 21 nt, synthetic construct
  source 1..21: mol_type = other RNA
accttcgccaactattctaca

SEQ ID NO: 40 - RNA, 23 nt, synthetic construct
  source 1..23: mol_type = other RNA
tgtagaatagttggcgaaggtct

SEQ ID NO: 41 - RNA, 21 nt, synthetic construct
  source 1..21: mol_type = other RNA
ccgcaagaggttctttaggga

SEQ ID NO: 42 - RNA, 23 nt, synthetic construct
  source 1..23: mol_type = other RNA
tccctaaagaacctcttgcgggt

SEQ ID NO: 43 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = um
  modified_base 4: mod_base = gm
  modified_base 5: mod_base = cm
  modified_base 6: mod_base = um
  modified_base 7: mod_base = cm
  modified_base 8: mod_base = gm
  modified_base 9: mod_base = OTHER; note = 2'-fluoroadenosine
  modified_base 10: mod_base = OTHER; note = 2'-fluorocytidine
  modified_base 11: mod_base = OTHER; note = 2'-fluorouridine
  modified_base 12: mod_base = cm
  modified_base 13: mod_base = um
  modified_base 14: mod_base = OTHER; note = 2'-O-methyladenosine
  modified_base 15: mod_base = OTHER; note = 2'-O-methyladenosine
  modified_base 16: mod_base = cm
  modified_base 17: mod_base = cm
  modified_base 18: mod_base = gm
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
gttgctcgactctaaccgcta

SEQ ID NO: 44 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = gm
  modified_base 4: mod_base = cm
  modified_base 5: mod_base = OTHER; note = 2'-fluoroguanosine
  modified_base 6: mod_base = gm
  modified_base 7: mod_base = um
  modified_base 8: mod_base = OTHER; note = 2'-fluorouridine
  modified_base 9: mod_base = OTHER; note = 2'-O-methyladenosine
  modified_base 10: mod_base = gm
  modified_base 11: mod_base = OTHER; note = 2'-O-methyladenosine
  modified_base 12: mod_base = gm
  modified_base 13: mod_base = um
  modified_base 14: mod_base = OTHER; note = 2'-fluorocytidine
  modified_base 15: mod_base = gm
  modified_base 16: mod_base = OTHER; note = 2'-fluoroadenosine
  modified_base 17: mod_base = gm
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methylguanosine with a 3'-phosphorothioate linkage
  modified_base 23: mod_base = gm
tagcggttagagtcgagcaacgg

SEQ ID NO: 45 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methyluridine with a 3'-phosphorothioate linkage
  modified_base 3: mod_base = cm
  modified_base 4: mod_base = gm
  modified_base 5: mod_base = cm
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluoroadenosine
  modified_base 12: mod_base = um
  modified_base 13: mod_base = um
  modified_base 14: mod_base = cm
  modified_base 15: mod_base = um
  modified_base 16: mod_base = OTHER; note = 2'-O-methyladenosine
  modified_base 17: mod_base = cm
  modified_base 18: mod_base = gm
  modified_base 19: mod_base = OTHER; note = 2'-O-methyluridine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
ttcgccaactattctacgtga

SEQ ID NO: 46 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = cm
  modified_base 5: mod_base = OTHER; note = 2'-fluoroguanosine
  modified_base 6: mod_base = um
  modified_base 7: mod_base = OTHER; note = 2'-O-methyladenosine
  modified_base 8: mod_base = OTHER; note = 2'-fluoroguanosine
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = OTHER; note = 2'-O-methyladenosine
  modified_base 13: mod_base = gm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = gm
  modified_base 18: mod_base = cm
  modified_base 19: mod_base = gm
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methylguanosine with a 3'-phosphorothioate linkage
  modified_base 23: mod_base = gm
tcacgtagaatagttggcgaagg

SEQ ID NO: 47 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylcytidine with a 3'-phosphorothioate linkage
  modified_base 3: mod_base = cm
  modified_base 4: mod_base = um
  modified_base 5: mod_base = um
  modified_base 6: mod_base = cm
  modified_base 7: mod_base = gm
  modified_base 8: mod_base = cm
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluoroadenosine
  modified_base 12: mod_base = cm
  modified_base 13: mod_base = um
  modified_base 14: mod_base = OTHER; note = 2'-O-methyladenosine
  modified_base 15: mod_base = um
  modified_base 16: mod_base = um
  modified_base 17: mod_base = cm
  modified_base 18: mod_base = um
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
accttcgccaactattctaca

SEQ ID NO: 48 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = um
  modified_base 4: mod_base = OTHER; note = 2'-O-methyladenosine
  modified_base 5: mod_base = OTHER; note = 2'-fluoroguanosine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluorouridine
  modified_base 9: mod_base = OTHER; note = 2'-O-methyladenosine
  modified_base 10: mod_base = gm
  modified_base 11: mod_base = um
  modified_base 12: mod_base = um
  modified_base 13: mod_base = gm
  modified_base 14: mod_base = OTHER; note = 2'-fluoroguanosine
  modified_base 15: mod_base = cm
  modified_base 16: mod_base = OTHER; note = 2'-fluoroguanosine
  modified_base 17: mod_base = OTHER; note = 2'-O-methyladenosine
  modified_base 18: mod_base = OTHER; note = 2'-O-methyladenosine
  modified_base 19: mod_base = gm
  modified_base 20: mod_base = gm
  modified_base 21: mod_base = OTHER; note = 2'-O-methyluridine with a 3'-phosphorothioate linkage
  modified_base 22: mod_base = OTHER; note = 2'-O-methylcytidine with a 3'-phosphorothioate linkage
  modified_base 23: mod_base = um
tgtagaatagttggcgaaggtct

SEQ ID NO: 49 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroguanosine
  modified_base 11: mod_base = OTHER; note = 2'-fluorouridine
  modified_base 12: mod_base = um
  modified_base 13: mod_base = cm
  modified_base 14: mod_base = um
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
ccgcaagaggttctttaggga

SEQ ID NO: 50 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = cm
  modified_base 4: mod_base = cm
  modified_base 5: mod_base = OTHER; note = 2'-fluorouridine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluoroadenosine
  modified_base 9: mod_base = gm
  modified_base 10: mod_base = OTHER; note = 2'-O-methyladenosine
  modified_base 11: mod_base = OTHER; note = 2'-O-methyladenosine
  modified_base 12: mod_base = cm
  modified_base 13: mod_base = cm
  modified_base 14: mod_base = OTHER; note = 2'-fluorouridine
  modified_base 15: mod_base = cm
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = um
tccctaaagaacctcttgcgggt

SEQ ID NO: 51 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = cm
  modified_base 5: mod_base = OTHER; note = 2'-fluoroguanosine
  modified_base 6: mod_base = um
  modified_base 7: mod_base = OTHER; note = 2'-fluoroadenosine
  modified_base 8: mod_base = gm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = OTHER; note = 2'-O-methyladenosine
  modified_base 13: mod_base = gm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = gm
  modified_base 18: mod_base = cm
  modified_base 19: mod_base = gm
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methylguanosine with a 3'-phosphorothioate linkage
  modified_base 23: mod_base = gm
tcacgtagaatagttggcgaagg

SEQ ID NO: 52 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = OTHER; note = 2'-fluoroadenosine
  modified_base 4: mod_base = cm
  modified_base 5: mod_base = gm
  modified_base 6: mod_base = um
  modified_base 7: mod_base = OTHER; note = 2'-fluoroadenosine
  modified_base 8: mod_base = gm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = OTHER; note = 2'-O-methyladenosine
  modified_base 13: mod_base = gm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = gm
  modified_base 18: mod_base = cm
  modified_base 19: mod_base = gm
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methylguanosine with a 3'-phosphorothioate linkage
  modified_base 23: mod_base = gm
tcacgtagaatagttggcgaagg

SEQ ID NO: 53 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = cm
  modified_base 5: mod_base = gm
  modified_base 6: mod_base = um
  modified_base 7: mod_base = OTHER; note = 2'-O-methyladenosine
  modified_base 8: mod_base = gm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = OTHER; note = 2'-O-methyladenosine
  modified_base 13: mod_base = gm
  modified_base 14: mod_base = OTHER; note = 2'-fluorouridine
  modified_base 15: mod_base = um
  modified_base 16: mod_base = gm
  modified_base 17: mod_base = gm
  modified_base 18: mod_base = cm
  modified_base 19: mod_base = gm
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methylguanosine with a 3'-phosphorothioate linkage
  modified_base 23: mod_base = gm
tcacgtagaatagttggcgaagg

SEQ ID NO: 54 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = gm
  modified_base 4: mod_base = cm
  modified_base 5: mod_base = OTHER; note = 2'-fluoroguanosine
  modified_base 6: mod_base = gm
  modified_base 8: mod_base = um
  modified_base 7: mod_base = OTHER; note = 2'-fluorouridine
  modified_base 9: mod_base = OTHER; note = 2'-O-methyladenosine
  modified_base 10: mod_base = gm
  modified_base 11: mod_base = OTHER; note = 2'-O-methyladenosine
  modified_base 12: mod_base = gm
  modified_base 13: mod_base = um
  modified_base 14: mod_base = OTHER; note = 2'-fluorocytidine
  modified_base 15: mod_base = gm
  modified_base 16: mod_base = OTHER; note = 2'-fluoroadenosine
  modified_base 17: mod_base = gm
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methylguanosine with a 3'-phosphorothioate linkage
  modified_base 23: mod_base = gm
tagcggttagagtcgagcaacgg

SEQ ID NO: 55 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = OTHER; note = 2'-fluoroguanosine
  modified_base 4: mod_base = cm
  modified_base 5: mod_base = gm
  modified_base 6: mod_base = gm
  modified_base 8: mod_base = um
  modified_base 7: mod_base = OTHER; note = 2'-fluorouridine
  modified_base 9: mod_base = OTHER; note = 2'-O-methyladenosine
  modified_base 10: mod_base = gm
  modified_base 11: mod_base = OTHER; note = 2'-O-methyladenosine
  modified_base 12: mod_base = gm
  modified_base 13: mod_base = um
  modified_base 14: mod_base = OTHER; note = 2'-fluorocytidine
  modified_base 15: mod_base = gm
  modified_base 16: mod_base = OTHER; note = 2'-fluoroadenosine
  modified_base 17: mod_base = gm
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methylguanosine with a 3'-phosphorothioate linkage
  modified_base 23: mod_base = gm
tagcggttagagtcgagcaacgg

SEQ ID NO: 56 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = gm
  modified_base 4: mod_base = cm
  modified_base 5: mod_base = gm
  modified_base 6: mod_base = gm
  modified_base 7: mod_base = um
  modified_base 8: mod_base = um
  modified_base 9: mod_base = OTHER; note = 2'-O-methyladenosine
  modified_base 10: mod_base = gm
  modified_base 11: mod_base = OTHER; note = 2'-O-methyladenosine
  modified_base 12: mod_base = gm
  modified_base 13: mod_base = um
  modified_base 14: mod_base = OTHER; note = 2'-fluorocytidine
  modified_base 15: mod_base = gm
  modified_base 16: mod_base = OTHER; note = 2'-O-methyladenosine
  modified_base 17: mod_base = gm
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methylguanosine with a 3'-phosphorothioate linkage
  modified_base 23: mod_base = gm
tagcggttagagtcgagcaacgg

SEQ ID NO: 57 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = cm
  modified_base 4: mod_base = cm
  modified_base 5: mod_base = OTHER; note = 2'-fluorouridine
  modified_base 6: mod_base = OTHER; note = 2'-O-methyladenosine
  modified_base 8: mod_base = OTHER; note = 2'-O-methyladenosine
  modified_base 7: mod_base = OTHER; note = 2'-fluoroadenosine
  modified_base 9: mod_base = gm
  modified_base 10: mod_base = OTHER; note = 2'-O-methyladenosine
  modified_base 11: mod_base = OTHER; note = 2'-O-methyladenosine
  modified_base 12: mod_base = cm
  modified_base 13: mod_base = cm
  modified_base 14: mod_base = OTHER; note = 2'-fluorouridine
  modified_base 15: mod_base = cm
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = um
tccctaaagaacctcttgcgggt

SEQ ID NO: 58 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = OTHER; note = 2'-fluorocytidine
  modified_base 4: mod_base = cm
  modified_base 5: mod_base = um
  modified_base 6: mod_base = OTHER; note = 2'-O-methyladenosine
  modified_base 8: mod_base = OTHER; note = 2'-O-methyladenosine
  modified_base 7: mod_base = OTHER; note = 2'-fluoroadenosine
  modified_base 9: mod_base = gm
  modified_base 10: mod_base = OTHER; note = 2'-O-methyladenosine
  modified_base 11: mod_base = OTHER; note = 2'-O-methyladenosine
  modified_base 12: mod_base = cm
  modified_base 13: mod_base = cm
  modified_base 14: mod_base = OTHER; note = 2'-fluorouridine
  modified_base 15: mod_base = cm
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = um
tccctaaagaacctcttgcgggt

SEQ ID NO: 59 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = cm
  modified_base 4: mod_base = cm
  modified_base 5: mod_base = um
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-O-methyladenosine
  modified_base 9: mod_base = gm
  modified_base 10: mod_base = OTHER; note = 2'-O-methyladenosine
  modified_base 11: mod_base = OTHER; note = 2'-O-methyladenosine
  modified_base 12: mod_base = cm
  modified_base 13: mod_base = cm
  modified_base 14: mod_base = OTHER; note = 2'-fluorouridine
  modified_base 15: mod_base = cm
  modified_base 16: mod_base = um
  modified_base 17: mod_base = um
  modified_base 18: mod_base = gm
  modified_base 19: mod_base = cm
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = um
tccctaaagaacctcttgcgggt

SEQ ID NO: 60 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = gm
  modified_base 4: mod_base = cm
  modified_base 6: mod_base = OTHER; note = 2'-fluoroguanosine
  modified_base 5: mod_base = gm
  modified_base 7: mod_base = um
  modified_base 8: mod_base = um
  modified_base 9: mod_base = OTHER; note = 2'-O-methyladenosine
  modified_base 10: mod_base = gm
  modified_base 11: mod_base = OTHER; note = 2'-O-methyladenosine
  modified_base 12: mod_base = gm
  modified_base 13: mod_base = um
  modified_base 14: mod_base = OTHER; note = 2'-fluorocytidine
  modified_base 15: mod_base = gm
  modified_base 16: mod_base = OTHER; note = 2'-fluoroadenosine
  modified_base 17: mod_base = gm
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methylguanosine with a 3'-phosphorothioate linkage
  modified_base 23: mod_base = gm
tagcggttagagtcgagcaacgg

SEQ ID NO: 61 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = cm
  modified_base 5: mod_base = gm
  modified_base 6: mod_base = OTHER; note = 2'-fluorouridine
  modified_base 7: mod_base = OTHER; note = 2'-O-methyladenosine
  modified_base 8: mod_base = gm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = OTHER; note = 2'-O-methyladenosine
  modified_base 13: mod_base = gm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = gm
  modified_base 18: mod_base = cm
  modified_base 19: mod_base = gm
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methylguanosine with a 3'-phosphorothioate linkage
  modified_base 23: mod_base = gm
tcacgtagaatagttggcgaagg

SEQ ID NO: 62 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = um
  modified_base 4: mod_base = OTHER; note = 2'-O-methyladenosine
  modified_base 5: mod_base = gm
  modified_base 6: mod_base = OTHER; note = 2'-fluoroadenosine
  modified_base 7: mod_base = OTHER; note = 2'-O-methyladenosine
  modified_base 8: mod_base = um
  modified_base 9: mod_base = OTHER; note = 2'-O-methyladenosine
  modified_base 10: mod_base = gm
  modified_base 11: mod_base = um
  modified_base 12: mod_base = um
  modified_base 13: mod_base = gm
  modified_base 14: mod_base = OTHER; note = 2'-fluoroguanosine
  modified_base 15: mod_base = cm
  modified_base 16: mod_base = OTHER; note = 2'-fluoroguanosine
  modified_base 17: mod_base = OTHER; note = 2'-O-methyladenosine
  modified_base 18: mod_base = OTHER; note = 2'-O-methyladenosine
  modified_base 19: mod_base = gm
  modified_base 20: mod_base = gm
  modified_base 21: mod_base = OTHER; note = 2'-O-methyluridine with a 3'-phosphorothioate linkage
  modified_base 22: mod_base = OTHER; note = 2'-O-methylcytidine with a 3'-phosphorothioate linkage
  modified_base 23: mod_base = um
tgtagaatagttggcgaaggtct

SEQ ID NO: 63 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = cm
  modified_base 4: mod_base = cm
  modified_base 5: mod_base = um
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-O-methyladenosine
  modified_base 9: mod_base = gm
  modified_base 10: mod_base = OTHER; note = 2'-O-methyladenosine
  modified_base 11: mod_base = OTHER; note = 2'-O-methyladenosine
  modified_base 12: mod_base = cm
  modified_base 13: mod_base = cm
  modified_base 14: mod_base = OTHER; note = 2'-fluorouridine
  modified_base 15: mod_base = cm
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = um
tccctaaagaacctcttgcgggt

SEQ ID NO: 64 - RNA, 10277 nt, Homo sapiens
  source 1..10277: mol_type = mRNA
atctcccagctcagccgagcccgtgcccaggccacgctttgttccagccgccgcctcctctaccctacggcgtccggagccatccctcgcctgctcgctctctcctttcgcccactccctgcatctgggcctgcatcacctttgccaaccgctcccccgatcctgccgacactcctcccccaaacttctgaccggcacccttgcctggtacccttctctccattcctccccctccatcttctttccccgacccctctcgggtccctcttttcccaaaacccgggtctctccgcgtggccccgcctccaggccggggatgtcccccgcggccccgcgcccatggtcctgacgctgcttctctccgcctacaagctgtgtcgcttcttcgccatgtcgggcccacggccgggcgccgagcggctggcggtgcctgggccagatgggggcggtggcacgggcccatggtgggctgcgggtggccgcgggccccgcgaagtgtcgccgggggcaggcaccgaggtgcaggacgccctggagcgcgcgctgccggagctgcagcaggccttgtccgcgctgaagcaggcgggcggcgcgcgggccgtgggcgccggcctggccgaggtcttccaactggtggaggaggcctggctgctgccggccgtgggccgcgaggtagcccagggtctgtgcgacgccatccgcctcgatggcggcctcgacctgctgttgcggctgctgcaggcgccggagttggagacgcgtgtgcaggccgcgcgcctgctggagcagatcctggtggctgagaaccgagaccgcgtggcgcgcattgggctgggcgtgatcctgaacctggcgaaggaacgcgaacccgtagagctggcgcggagcgtggcaggcatcttggagcacatgttcaagcattcggaggagacatgccagaggctggtggcggccggcggcctggacgcggtgctgtattggtgccgccgcacggaccccgcgctgctgcgccactgcgcgctggcgctgggcaactgcgcgctgcacgggggccaggcggtgcagcgacgcatggtagagaagcgcgcagccgagtggctcttcccgctcgccttctccaaggaggacgagctgcttcggctgcacgcctgcctcgcagtagcggtgttggcgactaacaaggaggtggagcgcgaggtggagcgctcgggcacgctggcgctcgtggagccgcttgtggcctcgctggaccctggccgcttcgcccgctgtctggtggacgccagcgacacaagccagggccgcgggcccgacgacctgcagcgcctcgtgccgttgctcgactctaaccgcttggaggcgcagtgcatcggggctttctacctctgcgccgaggctgccatcaagagcctgcaaggcaagaccaaggtgttcagcgacatcggcgccatccagagcctgaaacgcctggtttcctactctaccaatggcactaagtcggcgctggccaagcgcgcgctgcgcctgctgggcgaggaggtgccacggcccatcctgccctccgtgcccagctggaaggaggccgaggttcagacgtggctgcagcagatcggtttctccaagtactgcgagagcttccgggagcagcaggtggatggcgacctgcttctgcggctcacggaggaggaactccagaccgacctgggcatgaaatcgggcatcacccgcaagaggttctttagggagctcacggagctcaagaccttcgccaactattctacgtgcgaccgcagcaacctggcggactggctgggcagcctggacccgcgcttccgccagtacacctacggcctggtcagctgcggcctggaccgctccctgctgcaccgcgtgtctgagcagcagctgctggaagactgcggcatccacctgggcgtgcaccgcgcccgcatcctcacggcggccagagaaatgctacactccccgctgccctgtactggtggcaaacccagtggggacactccagatgtcttcatcagctaccgccggaactcaggttcccagctggccagtctcctgaaggtgcacctgcagctgcatggcttcagtgtcttcattgatgtggagaagctggaagcaggcaagttcgaggacaaactcatccagagtgtcatgggtgcccgcaactttgtgttggtgctatcacctggagcactggacaagtgcatgcaagaccatgactgcaaggattgggtgcataaggagattgtgactgctttaagctgcggcaagaacattgtgcccatcattgatggcttcgagtggcctgagccccaggtcctgcctgaggacatgcaggctgtgcttactttcaacggtatcaagtggtcccacgaataccaggaggccaccattgagaagatcatccgcttcctgcagggccgctcctcccgggactcatctgcaggctctgacaccagtttggagggtgctgcacccatgggtccaacctaaccagtccccagttccccagccctgctgtgacttccatttccatcgtcctttctgaaggaacagctcctgaaaccagtctccctgggctgagacaacctgggctcttcttaggaaatggctctccctccccctgtcccccaccctcatggcccacctccaacccactttcctcagtatctggagagggaagggaagtcaggcttgggcacgggaggttagaactcccccaggccctgccattgggttgtctgtctccgtcatggggagggtccctgctcagttctggagacactggagttggggtgggggtggttctgcattcccttctcctgctgatagcagtcagcttgaggaggatgacggaaggcagcctcagacaggaattaaggcaatgcccaggcgggcctgggcactgtattctgagcaagggcctgggcccaggagccagccagggatgagtgccatcatggctctccactcagactgtgcctggcccctgcacttacaacttcctgccgctctgtggccttgccctgtaatcactcagtgcccttagctagcctgactaagtcccagatcccctacagcttccttcggtgtggtatcttttgccacatccagggcgagggttgaggcaaaccagccctccctctgacttccttgtcactgcagccagctttgctgcacttgctggtgcacaggagcctcctgtttgggcctgggtctgggcatggggaggccgtgcctcaaagcccaccctaccccatgccttggtgctgtgcctcaggctccttcctggtctggcccagctggcttccccagcccctcagccatccagggctacccactgcttactcagggaccaggcagcccccatggcagtaaaagcagcctagacagaacctgcagctctgtggaaagaggcaaagtcctgaaaaggcaaagggttgtcacttagggcagcttctccaactttaacatgcatccaagtcacctgggaatgttgttaaaatcaggagatctggggtggggcctaggactctgcatttcttacagattcccaggtgagctgatgctggtggttaagggtagcaaatctctaaagcacgaagccctcacaaatctttgccatttcccaaacactccgctccatggtctccagtcatcagagcaactctacctggtattatcatccccattttacagataatgacactgaggctcagaaaggttgaggataagcccactttcctgtcattagtggcagccccagatccagacctaggcctcctggcacccagtccactggcagtggaattgctttcctgagaatcattctgaggctgggctattgcttctcccttgcttcaaagaatctagcagcgggggataggattttgcaacaaaaagctgacccagaggccatacagagcaggaatatcccattgccccctcctccactgggttcagagggtaagaaagcaccctccaataaacccaggctccaggccgtgggggctgctgaaggctctttccccgcaagggccaggtgttgacaccttaaagctggctgcgcccccagccccactcttggctgtgctggccaggtgactcctagttcttggccacatcatcagaaagtcaaaggtctcactccaggtttggggctccttccttccactcccctccctgccagagtctgtcttggccagtgccagcctcgatgctttggttttgaccccacctgatcctcctttcctcatgcagcacaagtgctcaccggggccagagccagggcatggatatgacaagcagggcagcctggacactgccctcacaggacagcgccaataacaatacagtgtctgagtatctccaggggatgatttctggctctttgtctccaatcagtcccactccctcctgaggtccccaagggcagtattcagagaggtttcctgcgttttatttctatttggtataccctccactgttgtccactgccctgtgtggccttctggttgacctctgcccgatcttctgtctctctgagggaatcagagtccagcatccagccccagctggaacagctgaagtcacaagcctcctctaagccaaggccagtgtgttcagaggtgactgccacccatactaggacaaacacagctcagatcaccaggtcaagcacctaggcctggcttctcctgagacagaggactcagaagtggcctttcctccaaagcctgctcagacacaggtctgtagggccagggtgttctgcttggctgggctgcagctgctacccctcggttggggctgagtcagccagatcctccccctacttctccccaagggccaagaactgctcagggacattaaaggtcaaaagtccagccacactcattcatcctttccccaggcccatgaagagaggcatctcattgtagaatgtatgaggaagtgggaagtatctcagagaatcagctaagtttcctaacttgtccatccaaatgtgatcaccacgattcaacaatttggggcattgctgatctagccgttcctagtggggcttgctcaaggttgcacagcgagtcagtagaagccctggctggccccacttggtaccaatccaccaggcagctcagggctcctgcccagcccagcagcttctgttgtctaacgtatggcaggcagactgggagcaggaaaacagagggccccaaagcccaaggcaccagaaggtttgtttcagtttgctgaagctgatttgtaatgattggcactcttcagccaggggagtgggtaggccatagccaaggatcgattccccaaccacagcaaaggcaacactcttcctccagagatcaccaagcccctcttacctccctccctccttcccaaggctggcactaaccaggtaccacattcattgttaaggaatggctgatgactgctacacgtgttgggaacctggttggggctgtgcagtttgggctggaaggagagatgccagccctcgtgctgcctctggtccctgaagtgtcacctctctcaggacctctcctctggcctgtggggttataagtgatggatagcagaaagggagaactgactcctgtcccaaatagctcctctgccacctgtcctgcagtgggcctgtgtgggttatgattctagatcctagacagaggctgggtcagctgtggatggggtggtgccttggtctctcttgactacctcgtccaaagagagcactgcccttagacaagagttgcttgtcctgctgtgggctgggcttccagctgcagacctccagttgcttggtgttcactttgctcctcttgccctctgtcttctggtccaggcagatcaggggctctggggaaactgctggaactcgaggtgaggatcagccttttccagcatcctgtgagagaccagagagagagtttggatttcatgtggggaaccctcaaggcctgtctggagaagtgacacaggatttactggggtgggctggtccaggtagctctcctgaacctcctccttccccaagctgagaagctgagagctggaggacaatatccagggacatggctctggaaaataacttttttttttttaagagacagggtcttgctctgttgtccaggctggagggcagtgacataatcatagctcactgtacccttgaactcctgggctcaagtgatcctcctgcctcagcctccttagtagctgggactaccagtgcataccaccatgcctgggtgattttttaaattttttatacagacaaggtcttgctatgttgcccaggctgatcttgaattcccgggctcaagtggtcctcctgcctcagcctcccacaggatcgggattacaggcaagagcctccacgcccggccatgaaatataattcttaatatcatacaggaaaaagtcagcgggtcaagctagcctgtggcccagccacaactagctgacaaagcttcctggccttccctttaacacagttctgctgccatagttccatctataaaatgggaatggagggaaataggggaactgggagagagaacacagccttgccaagcagcaatgttagcctgatccttcctccacctagctcgccatctcgcccttggaaaatggctcctggaggattaggcagccatctgcaaggagaggggcaacctgggacaagacacccagagggtaaggattccaggaatgaagctgccatttctggttgggaggagaagaggaaacttttaagagaaagggctccattatgagcatgggttcagggccctgcattacccaatcagaacagccgggatgagcaggaggccagctcccaggaggaaggggaaccccttcataaagttcagagtggctgggtagagtgagttgaagatgccggaggccgtcagcatggccaggctattcacacaggccacagcagaaaagagagcacctgtgaagaaataaataccatactctggagtccgaaagggccatattccaactctggcaccaccacctcacagctgtgtgaccgggagtagtcacttaacctatgtctccccttcctcaccagtaaatcctgctacatcatgtactgtgacaaggattcagtaaggtcatatgtggacagtagctggcacagaggggctactaaacaaatggctgctattaaatccacattaaaagtacatgtgatctgacagaacccagcacataaaagaaaaaaaaagtacatgtgatattgtctgatgaaagcttgatggaaatggcttttttctggtttatcctctttggaatcatctcctgtttgggattaactgctggtctgatcagttccaatattcatagcggtgtcaccactgaatagcttcttatcctttgggttcctgttcctccttctgctaaataaggataatacctatttcctagattgtgagcaacattaagttcacatggaaatcacccatcactgggcctggtcccctggaagtagctagttagtaagggctgttcttttctcctgtttctcttgacatctctgggcacagagaaagtgctgggaaaaaaagtttaggtgaatgaatgaagacacatggattctggggacaccagaacccacagtgggctctgtatggcaccagagtctctgtcatcatcagatcctcattccaggacagatggaaaaagatgaatgtttccagactggggcataaagacccagaggctggagaagctgttctttatagatataccaggagaacccacagtttacaaaatgtgcaacaacccaacagaagttgagattaaattctgtcacatctagaggggtctgtgatgtcatcaaaagcaaaccacccacatcacagatgaagaaacaggcctgtggcagggctcggactaaaacccagatcctgagaccagctgcttttaaacacagacgtaggtttgcatcctagctccaccatttactgagtaaccttgggtgagccaatgtaaccccctgggtctctgtttctttatctgtcaactgtggaaaatgaaacccatgtcacaaggttgttcacttctgggcttgtacacgctgaccccagagaaacagggaactctggcatcaccacacccatcttacagacggaaaagctgaggtctgcagagagtaaatcctctgctctggttatctagaaagaacataattgtgctctgctgactgcaaatcccaactctgcggtttgaaaatccaaggtggcatgatcctctgcccattgtgggcaatttcacagaaatgtgtttgttttggccacttacttctccagggtgagaggggggaaggcaagctgttcccccagccatggctgcccatcagcccgtttcgggcagcactggacatgaggaaccagacacaggtgggttctgacactcaccctgctctgtctctctcaccagcttggagagtttagcccggatgacaggtgtgatgactaatgacaggaaaagcaacccatatcctgtggagaaacaaacactcatcaggaaaatggggctggggagaggggcgtccaagggaaaggcagcagagctcctatccataccccacgtggggcttaggttagacccaggaagaacttccttgatggtgagggtgggaagacagtagtcaaggaggaatggagactgcccttgtctgggcttggccacctgctagctctcatgaatgaatgctaattcccattgattgctttcttgtctgaacctcttgtggtcacagcaggcatcacccacccacttggcacttagtagggatatggcagggcacagaaaacaagcatgggctttggagtcagccctgagttcaaaacctgatgccattacatattatctgtgtggcctggggtacttaccctctctgatcctgactccctgtatgaggaagataataaggccttcatcacaggatggttctgaggcataggaggctgaataatggtgcccaatggcatcagattcatagccctggaacctgtaaatactaccttatttggaaaatgagtctatgcaggtgtgcagttaagcctcctgagagagcagagttatcctggattaggttgggccctaaatgccgtcacacatatctttataagaggaaagcagacggagatttggcaccgacagaattgagaaggcacaaagaggaggagagtcaatgtgagcacagaggcagagactggtgatggccgccccaagccaaggaatgccagcagccccagaagctggaagaaatgagaaacacgttctctcctggaggcttgcaagggagcactgcctgctgactgcttccattcagcccggtggtactgactttggacttctggcctccagaactgtgagagaatatgtttctgttgtgttaagcccccaagtttgtggtatgtcattacagcaatctcagggaaccaatacatgaggtaaaaaggtaacatctatgaagagcatggcatagggacacagcaaatgggagttccttttccctttgcattcagttacttacaggcttcctgttttcttcataaccatttctctccctgtgcgactgctgactcctcagcaaaactgcaaactcctacaggacagtggatcctccaaagaaggtatacgatgaggcatccagggaccctagcagtgtcaggcccctcaaatcccactctgttgagacctccccccgacccagagcaatgacagcatctttatcatctctgcatcccccagggccatcagcaggagggaaaggttcccttctgcttaattgtcagacaagcagttgagttaagaaatctgtgattattgtattgttgactatacacagcacattttagggctctatcaaaataaatctgtccctttaaaaaaagttaactaaagccgggcacggtggctcatgcctgtaatcccaacactttgggaggctgaggcaggcggatccttgagctcaggagttagagacctggactgggcaaaatggtgaggaccccatctctataaaaaatacaaaaattagcaaggtgtggtaatgtgcaccagtggtcccagctactagagaggccaaggtgggaggatcatctgggcccgggggatgaggctgcagtgagccatgatcgtgccactgcactctagcctgggtaacaaagcgagaccctgtctctaaatacatcaatcaaataaaaattttaaaaagttaa

SEQ ID NO: 65 - protein, 724 aa, Homo sapiens
MVLTLLLSAYKLCRFFAMSGPRPGAERLAVPGPDGGGGTGPWWAAGGRGPREVSPGAGTEVQDALERALPELQQALSALKQAGGARAVGAGLAEVFQLVEEAWLLPAVGREVAQGLCDAIRLDGGLDLLLRLLQAPELETRVQAARLLEQILVAENRDRVARIGLGVILNLAKEREPVELARSVAGILEHMFKHSEETCQRLVAAGGLDAVLYWCRRTDPALLRHCALALGNCALHGGQAVQRRMVEKRAAEWLFPLAFSKEDELLRLHACLAVAVLATNKEVEREVERSGTLALVEPLVASLDPGRFARCLVDASDTSQGRGPDDLQRLVPLLDSNRLEAQCIGAFYLCAEAAIKSLQGKTKVFSDIGAIQSLKRLVSYSTNGTKSALAKRALRLLGEEVPRPILPSVPSWKEAEVQTWLQQIGFSKYCESFREQQVDGDLLLRLTEEELQTDLGMKSGITRKRFFRELTELKTFANYSTCDRSNLADWLGSLDPRFRQYTYGLVSCGLDRSLLHRVSEQQLLEDCGIHLGVHRARILTAAREMLHSPLPCTGGKPSGDTPDVFISYRRNSGSQLASLLKVHLQLHGFSVFIDVEKLEAGKFEDKLIQSVMGARNFVLVLSPGALDKCMQDHDCKDWVHKEIVTALSCGKNIVPIIDGFEWPEPQVLPEDMQAVLTFNGIKWSHEYQEATIEKIIRFLQGRSSRDSSAGSDTSLEGAAPMGPT

SEQ ID NO: 66 - protein, 681 aa, Mus musculus
HHHHHHCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 67 - protein, 678 aa, Homo sapiens
HHHHHHCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 68 - protein, 193 aa, Homo sapiens
HHHHHHHHGKPIPNPLLGLDSTGGGGSDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGGGGGGLPNIPVQTISRAAAEKLFGNMEGDCPSDWKTDSTCRMVTSESKNVKLTVS

(19) United States
(12) Patent Application Publication
(10) Pub. No.: US 2025/0249118 A1
(43) Pub. Date: Aug. 7, 2025

(54) SARM1 RNAi AGENTS

(71) Applicant: ELI LILLY AND COMPANY, Indianapolis, IN (US)

(72) Inventors: Riazul ALAM, Fishers, IN (US); Nicholas A. BABB, Noblesville, IN (US); Johnny Eugene CROY, Fishers, IN (US); Gregory Lawrence LACKNER, Indianapolis, IN (US); Yea Jin KAESER-WOO, Newton, MA (US)

(21) Appl. No.: 19/042,154

(22) Filed: Jan. 31, 2025

Related U.S. Application Data
(60) Provisional application No. 63/549,060, filed on Feb. 2, 2024.
(60) Provisional application No. 63/685,791, filed on Aug. 22, 2024.

(51) Int. Cl. A61K 47/68; A61K 9/00; A61P 25/28; C07K 16/28; C12N 15/113

(52) U.S. Cl. CPC A61K 47/6807; A61K 9/0019; A61K 47/6849; A61K 47/6889; A61P 25/28; C07K 16/2881; C12N 15/1137; C12N 2310/11; C12N 2310/14; C12N 2310/315; C12N 2310/321; C12N 2310/322; C12Y 302/02006

(57) ABSTRACT

Provided herein are SARM1 RNAi agents and compositions comprising a SARM1 RNAi agent. Also provided herein are methods of using the SARM1 RNAi agents or compositions comprising a SARM1 RNAi agent in reducing SARM1 expression and/or treating SARM1-mediated neurological diseases.

### SEQUENCE LISTING

The present application is being filed along with a Sequence Listing in ST.26 XML format. The Sequence Listing is provided as a file titled “30973_US” created 27 Jan. 2025 and is 293 kilobytes in size. The Sequence Listing information in the ST.26 XML format is incorporated herein by reference in its entirety.

### BACKGROUND

Axon degeneration is a pathological feature of many neurological diseases including neurodegenerative diseases, traumatic brain injury, and peripheral neuropathies (Gerdts et al., Science, 2015, 348:453-457). Damaged or unhealthy axons are eliminated by an intrinsic self-destruction program known as Wallerian degeneration, which is an active process of retrograde degeneration of the distal end of an axon while keeping the proximal axon segment and cell body intact (Gerdts, J., et al., Neuron, 2016, 89, 449-460; Whitmore, A. et al., Cell Death Differ., 2003, 10, 260-261).

SARM1 (sterile alpha and TIR motif containing 1) is a NAD+ hydrolase that plays an important role in axon degeneration. It has been reported that knocking-down or eliminating the expression of SARM1 leads to long-lasting protection of sensory neurons against injury-induced axonal degeneration (Gerdts et al., J Neurosci, 2013, 33, 13569-13580). WO2021/108602 describes SARM1 antisense oligonucleotides.

The blood brain barrier (BBB) is a selective semipermeable border of capillary endothelial cells that prevents solutes, including pathogens, from passing into the central nervous system (CNS). The BBB allows the passage of some small molecules by passive diffusion and the cells of BBB actively transport metabolic products crucial to neural function such as glucose and amino acids across the barrier using specific transport proteins. The BBB has neuroprotective function by tightly controlling access to the brain; but it also impedes access of therapeutic agents to CNS. Antibodies directed to transferrin receptor (“TfR”) have been used for modulating BBB transport. However, attempts at using anti-TfR antibodies to shuttle therapeutic agents across the BBB have proven challenging. To date, there are no approved TfR shuttles or conjugates for the treatment of CNS diseases.

RNA interference (RNAi) is a highly conserved regulatory mechanism in which RNA molecules are involved in sequence-specific suppression of gene expression by double-stranded RNA molecules (dsRNA) (Fire et al., Nature 391:806-811, 1998).

There remains a need for therapeutic agents that can inhibit or adjust the expression of SARM1 for treating SARM1-mediated neurological diseases e.g., by utilizing RNAi. There is also need for conjugates that can deliver SARM1 RNAi agent across the BBB into the CNS for the treatment of various CNS neurodegenerative diseases.

### SUMMARY OF INVENTION

Provided herein are SARM1 RNAi agents capable of crossing BBB and compositions comprising such a SARM1 RNAi agent. Also provided herein are methods of using such SARM1 RNAi agents or compositions comprising a SARM1 RNAi agent for reducing SARM1 expression, reducing axon degeneration, and/or treating SARM1-mediated neurological diseases in a subject.

In one aspect, provided herein are SARM1 RNAi agents comprising Formula (I): (R-L)ₙ-P, wherein R is a double stranded RNA (dsRNA) comprising a sense stand and an antisense strand, and the antisense strand is complementary to SARM1 mRNA; wherein L is a linker, or optionally absent; wherein P is a protein comprising one monovalent human TfR binding domain (“human TfR binding protein”); and wherein n is an integer of 1 to 3. In some embodiments, n is 1. In some embodiments, n is 2. In some embodiments, n is 3.

In some embodiments, provided herein are SARM1 RNAi agents comprising Formula (I): (R-L)ₙ-P, wherein R is a double stranded RNA (dsRNA) comprising a sense stand and an antisense strand, and the antisense strand is complementary to SARM1 mRNA; wherein L is a linker, or optionally absent; wherein P is a protein comprising one monovalent human TfR binding domain, wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6; and wherein n is an integer of 1 to 3. In some embodiments, n is 1. In some embodiments, n is 2. In some embodiments, n is 3. In some embodiments, VH comprises SEQ ID NO: 7 and VL comprises SEQ ID NO: 8. In some embodiments, VH comprises a sequence having at least 95% sequence identity to SEQ ID NO: 7 and VL comprises a sequence having at least 95% sequence identity to SEQ ID NO: 8. Exemplary sequences of human TfR binding domains and proteins are provided in Table 1a and 1b.

In some embodiments, L is a Mal-Tet-TCO linker, SMCC linker, GDM linker, MSPT linker or OD linker (see Table 4). In some embodiments, L is a SMCC linker in Table 4. In some embodiments, L is a MSPT linker in Table 4.

Exemplary unmodified sense strand and antisense strand sequences of dsRNA targeting human SARM1 mRNA are provided in Table 5. In some embodiments, the sense strand and the antisense strand of the dsRNA comprise a pair of nucleic acid sequences selected from the group consisting of:
(a) the sense strand comprises SEQ ID NO: 35, and the antisense strand comprises SEQ ID NO: 36;
(b) the sense strand comprises SEQ ID NO: 37, and the antisense strand comprises SEQ ID NO: 38;
(c) the sense strand comprises SEQ ID NO: 39, and the antisense strand comprises SEQ ID NO: 40; and
(d) the sense strand comprises SEQ ID NO: 41, and the antisense strand comprises SEQ ID NO: 42;
   wherein optionally one or more nucleotides of the sense strand and the antisense strand are independently modified nucleotides, and wherein optionally one or more internucleotide linkages of the sense strand and the antisense strand are modified internucleotide linkages. In some embodiments, the sense strand comprises SEQ ID NO: 35, and the antisense strand comprises SEQ ID NO: 36. In some embodiments, the sense strand comprises SEQ ID NO: 37, and the antisense strand comprises SEQ ID NO: 38. In some embodiments, the sense strand comprises SEQ ID NO: 39, and the antisense strand comprises SEQ ID NO: 40. In some embodiments, the sense strand comprises SEQ ID NO: 41, and the antisense strand comprises SEQ ID NO: 42.

The dsRNA can include modifications. The modifications can be made to one or more nucleotides of the sense and/or antisense strand or to the internucleotide linkages. In some embodiments, one or more nucleotides of the sense strand and/or the antisense strand are independently modified nucleotides, which means the sense strand and the antisense strand can have different modified nucleotides. In some embodiments, each nucleotide of the sense strand is a modified nucleotide. In some embodiments, each nucleotide of the antisense strand is a modified nucleotide. In some embodiments, the modified nucleotide is a 2′-fluoro modified nucleotide, 2′-O-methyl modified nucleotide, 2′ deoxy nucleotide (DNA), or 2′-O-alkyl (e.g., 2′-O—C₁₆ alkyl) modified nucleotide. In some embodiments, each nucleotide of the sense strand and the antisense strand is independently a modified nucleotide, e.g., a 2′-fluoro modified nucleotide, 2′-O-methyl modified nucleotide, 2′ deoxy nucleotide (DNA), or 2′-O-alkyl (e.g., 2′-O—C₁₆ alkyl) modified nucleotide.

In some embodiments, the sense strand has four 2′-fluoro modified nucleotides, e.g., at positions 7, 9, 10, 11 from the 5′ end of the sense strand. In some embodiments, at least one nucleotide of the sense strand is an unmodified RNA nucleotide. In some embodiments, at least one nucleotide of the sense strand is 2′ deoxy nucleotide (DNA). In some embodiments, the other nucleotides of the sense strand are 2′-O-methyl modified nucleotides. In some embodiments, the antisense strand has four 2′-fluoro modified nucleotides, e.g., at positions 2, 6, 14, 16 from the 5′ end of the antisense strand. In some embodiments, the other nucleotides of the antisense strand are 2′-O-methyl modified nucleotides.

In some embodiments, the sense strand has three 2′-fluoro modified nucleotides, e.g., at positions 9, 10, 11 from the 5′ end of the sense strand. In some embodiments, at least one nucleotide of the sense strand is an unmodified RNA nucleotide. In some embodiments, at least one nucleotide of the sense strand is 2′ deoxy nucleotide (DNA). In some embodiments, the other nucleotides of the sense strand are 2′-O-methyl modified nucleotides. In some embodiments, the antisense strand has five 2′-fluoro modified nucleotides, e.g., at positions 2, 5, 7, 14, 16 from the 5′ end of the antisense strand. In some embodiments, the antisense strand has five 2′-fluoro modified nucleotides, e.g., at positions 2, 5, 8, 14, 16 from the 5′ end of the antisense strand. In some embodiments, the antisense strand has five 2′-fluoro modified nucleotides, e.g., at positions 2, 3, 7, 14, 16 from the 5′ end of the antisense strand. In some embodiments, the antisense strand has three 2′-fluoro modified nucleotides, e.g., at positions 2, 14, 16 from the 5′ end of the antisense strand. In some embodiments, the other nucleotides of the antisense strand are 2′-O-methyl modified nucleotides.

In some embodiments, the 5′ end of the antisense strand has a phosphate analog, e.g., 5′-vinylphosphonate (5′-VP).

In some embodiments, the sense strand or the antisense strand comprises an abasic moiety or inverted abasic moiety.

In some embodiments, the sense strand and the antisense strand have one or more modified internucleotide linkages. In some embodiments, the modified internucleotide linkage is phosphorothioate linkage. In some embodiments, the sense strand has four or five phosphorothioate linkages. In some embodiments, the antisense strand has four or five phosphorothioate linkages. In some embodiments, the sense strand and the antisense strand each has four or five phosphorothioate linkages. In some embodiments, the sense strand has four phosphorothioate linkages and the antisense strand has five phosphorothioate linkages.

Exemplary modified sense strand and antisense strand sequences of dsRNA targeting human SARM1 mRNA are provided in Table 7.

In some embodiments, provided herein are SARM1 RNAi agents comprising Formula (I): (R-L)ₙ-P, wherein R is a double stranded RNA (dsRNA) comprising a sense stand and an antisense strand, and the antisense strand is complementary to SARM1 mRNA; wherein L is a linker, or optionally absent; wherein P is a protein comprising one monovalent human TfR binding domain, and P is selected from TBP1, TBP2, TBP3, TBP4, or TBP5 in Table 1b; and wherein n is an integer of 1 to 3. In some embodiments, n is 1. In some embodiments, n is 2. In some embodiments, n is 3. In some embodiments, L is a linker in Table 4 (e.g., a SMCC or MSPT linker in Table 4).

In some embodiments, provided herein are SARM1 RNAi agents comprising Formula (I): (R-L)ₙ-P, wherein R is a double stranded RNA (dsRNA) comprising a sense stand and an antisense strand, and the dsRNA is any dsRNA in Table 5 or 7; wherein L is a linker, or optionally absent; wherein P is a protein comprising one monovalent human TfR binding domain; and wherein n is an integer of 1 to 3. In some embodiments, n is 1. In some embodiments, n is 2. In some embodiments, n is 3. In some embodiments, L is a linker in Table 4 (e.g., a SMCC or MSPT linker in Table 4).

In some embodiments, provided herein are SARM1 RNAi agents comprising Formula (I): (R-L)ₙ-P, wherein R is a double stranded RNA (dsRNA) comprising a sense stand and an antisense strand, and the dsRNA is any dsRNA in Table 5 or 7; wherein L is a linker, or optionally absent; wherein P is a protein comprising one monovalent human TfR binding domain, and P is selected from TBP1, TBP2, TBP3, TBP4, or TBP5 in Table 1b; and wherein n is an integer of 1 to 3. In some embodiments, n is 1. In some embodiments, n is 2. In some embodiments, n is 3. In some embodiments, L is a linker in Table 4 (e.g., a SMCC or MSPT linker in Table 4).

In some embodiments, provided herein are SARM1 RNAi agents comprising Formula (I): (R-L)ₙ-P, wherein R is a double stranded RNA (dsRNA) comprising a sense stand and an antisense strand, and the antisense strand is complementary to SARM1 mRNA; wherein L is a linker, or optionally absent; wherein P is a protein comprising one monovalent human TfR binding domain; wherein the human TfR binding domain comprises two heavy chains HC1 and HC2 and one light chain LC1, wherein HC1 comprises SEQ ID NO: 14, LC1 comprises SEQ ID NO: 10, HC2 comprises SEQ ID NO: 15, and wherein n is 1 or 2. In some embodiments, n is 1. In some embodiments, n is 2. In some embodiments, L is a linker in Table 4 (e.g., a SMCC or MSPT linker in Table 4).

In some embodiments, provided herein are SARM1 RNAi agents comprising Formula (I): (R-L)ₙ-P, wherein R is a double stranded RNA (dsRNA) comprising a sense stand and an antisense strand, and the antisense strand is complementary to SARM1 mRNA; wherein L is a linker, or optionally absent; wherein P is a protein comprising one monovalent human TfR binding domain; wherein the human TfR binding domain comprises two heavy chains HC1 and HC2 and one light chain LC1, wherein HC1 comprises SEQ ID NO: 16, LC1 comprises SEQ ID NO: 10, HC2 comprises SEQ ID NO: 17, and wherein n is 1. In some embodiments, L is a linker in Table 4 (e.g., a SMCC or MSPT linker in Table 4).

In another aspect, provided herein are methods of treating a SARM1-mediated neurologic disease in a patient in need thereof, and such the method comprises administering to the patient an effective amount of the SARM1 RNAi agent or a pharmaceutical composition described herein. The SARM1 RNAi agent or a pharmaceutical composition comprising SARM1 RNAi agent can be administered to the patient intravenously or subcutaneously.

In another aspect, provided herein are SARM1 RNAi agents or pharmaceutical compositions comprising a SARM1 RNAi agent for use in a therapy. Also provided herein are SARM1 RNAi agents or pharmaceutical compositions comprising a SARM1 RNAi agent for use in the treatment of a SARM1-mediated neurological disease. Also provided herein are uses of the SARM1 RNAi agent in the manufacture of a medicament for treating a SARM1-mediated neurological disease.

### BRIEF DESCRIPTION OF THE DRAWINGS

FIG. 1A shows an exemplary preparative anion exchange (AEX) chromatogram of DAR profile for TBP5-SMCC-dsRNA No. 8 conjugate before purification.

FIG. 1B shows an exemplary analytical AEX chromatogram of DAR profile for TBP5-SMCC-dsRNA No. 8 conjugate after purification. FIG. 1C shows an exemplary analytical anion exchange (AEX) chromatogram of DAR profile for TBP5-SMCC-dsRNA No. 7 conjugate before purification. FIG. 1D shows an exemplary analytical AEX chromatogram of DAR profile for TBP5-SMCC-dsRNA No. 7 conjugate after purification. FIG. 1E shows an exemplary analytical anion exchange (AEX) chromatogram of DAR profile for TBP5-SMCC-dsRNA No. 6 conjugate before purification. FIG. 1F shows an exemplary analytical AEX chromatogram of DAR profile for TBP5-SMCC-dsRNA No. 6 conjugate after purification.

FIG. 2 shows in vitro SARM1 knockdown in SH-SY5Y cells with unconjugated dsRNA No. 8, cholesterol conjugated dsRNA No. 8 and TBP5 conjugated dsRNA No. 8.

FIGS. 3A and 3B show hTfR mouse proof of concept data demonstrating pharmacodynamic efficacy of TBP5-SARM1 dsRNA No. 8 conjugate with a single IV dosing at 10 mg/kg siRNA or four weekly IV doses at 10 mg/kg. SARM1 mRNA reduction in mouse hemibrain (3A) and gastrocnemius muscle (3B) are shown 28 days following initial dose. Both dosing groups demonstrated significant reduction of SARM1 mRNA in brain compared to non-dosed PBS groups. The error bars are Standard Deviations and statistical analysis was performed with a one-way Anova with Dunnett's multiple comparison test against PBS control group. Annotations indicate P values>0.0001=****; >0.001=***; >0.01=**; >0.05=*.

FIG. 4A shows reduction of SARM1 protein in Cynomolgus monkey tissues after single peripheral IV administration of TBP5-dsRNA No. 7 conjugate at 10 mg/k.

FIG. 4B shows reduction of SARM1 protein in Cynomolgus monkey tissues after single peripheral IV administration of TBP5-dsRNA No. 6 conjugate at 10 mg/kg siRNA.

FIG. 5 shows an exemplary analytical AEX chromatogram of DAR profile for TBP5-MSPT-dsRNA No. 8 conjugate after purification.

FIGS. 6A and 6B show hTfR transgenic mouse data demonstrating pharmacodynamic efficacy of TBP5-SMCC-dsRNA No. 8 or TBP5-MSPT-dsRNA No. 8 conjugate with a single IV dosing at 10 mg/kg siRNA. SARM1 mRNA reduction in mouse hemibrain (6A) and lumbar spinal cord (6B) are shown 28 days following initial dose. Both dosing groups demonstrated significant reduction of SARM1 mRNA in brain and spinal cord compared to the PBS groups. The error bars are Standard Deviations and statistical analysis was performed with a one-way Anova with Dunnett's multiple comparison test against PBS control group. Annotations indicate P values>0.0001=****; >0.001=***; >0.01=**; >0.05=*.

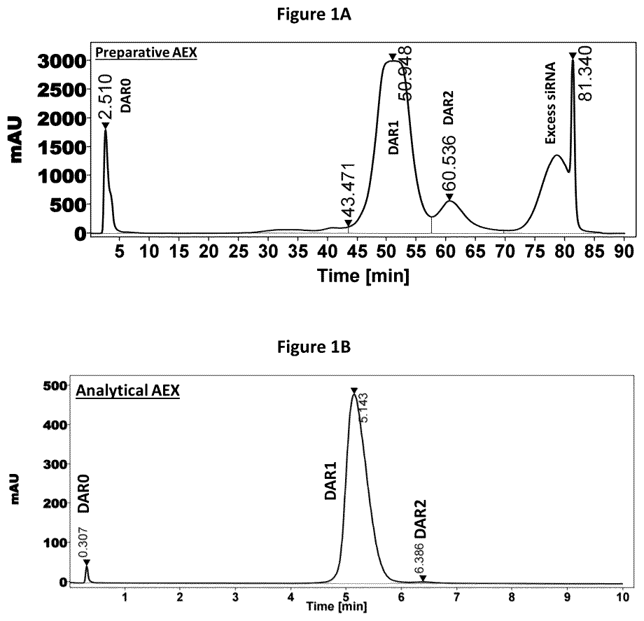

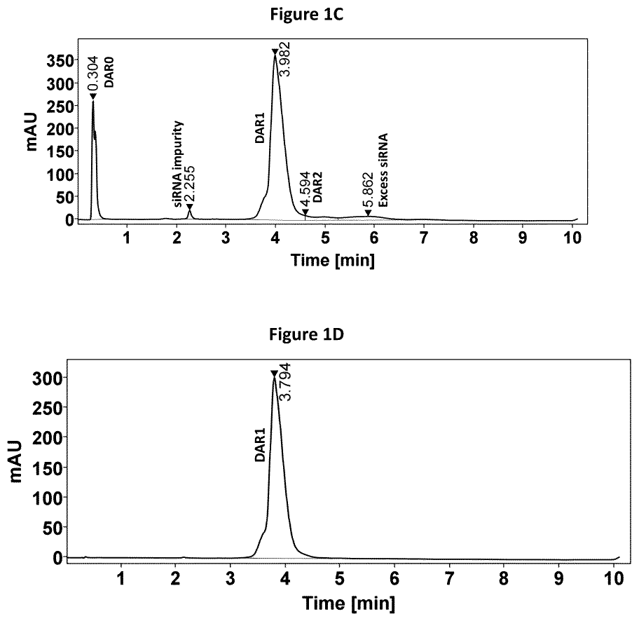

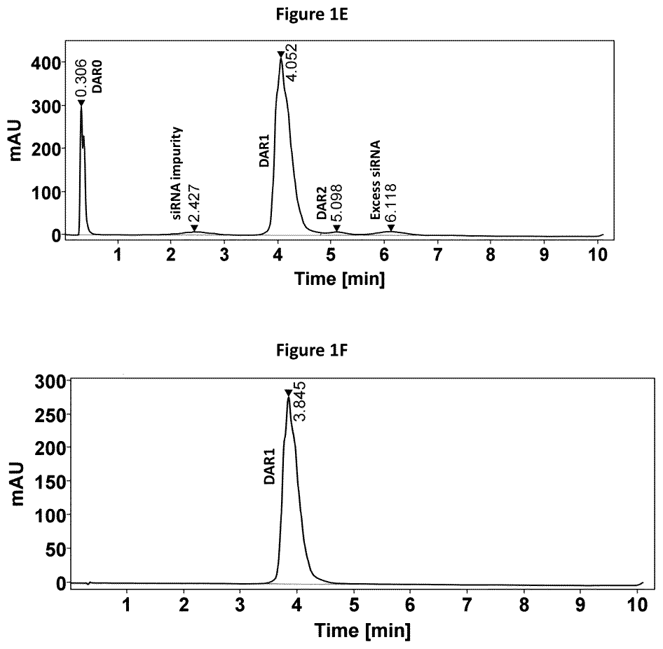

### DETAILED DESCRIPTION

Provided herein are SARM1 RNAi agents and compositions comprising a SARM1 RNAi agent. Also provided herein are methods of using the SARM1 RNAi agents or compositions comprising a SARM1 RNAi agent for reducing SARM1 expression, reducing axon degeneration, and/or treating SARM1-mediated neurological diseases in a subject.

In one aspect, provided herein are SARM1 RNAi agents comprising Formula (I): (R-L)ₙ-P, wherein R is a double stranded RNA (dsRNA) comprising a sense stand and an antisense strand, wherein the antisense strand is complementary to SARM1 mRNA; wherein L is a linker, or optionally absent; wherein P is a protein comprising one monovalent human TfR binding domain (“human TfR binding protein”); and wherein n is an integer of 1 to 3. In some embodiments, n is 1. In some embodiments, n is 2. In some embodiments, n is 3.

In some embodiments, provided herein are SARM1 RNAi agents comprising Formula (I): (R-L)ₙ-P, wherein R is a double stranded RNA (dsRNA) comprising a sense stand and an antisense strand, and the antisense strand is complementary to SARM1 mRNA; wherein L is a linker, or optionally absent; wherein P is a protein comprising one monovalent human TfR binding domain; wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6; and wherein n is an integer of 1 to 3. In some embodiments, n is 1. In some embodiments, n is 2. In some embodiments, n is 3. In some embodiments, L is a linker in Table 4 (e.g., a SMCC or MSPT linker in Table 4).

In some embodiments, provided herein are SARM1 RNAi agents comprising Formula (I): (R-L)ₙ-P, wherein R is a double stranded RNA (dsRNA) comprising a sense stand and an antisense strand, and the antisense strand is complementary to SARM1 mRNA; wherein L is a linker, or optionally absent; wherein P is a protein comprising one monovalent human TfR binding domain, and P is selected from TBP1, TBP2, TBP3, TBP4, or TBP5 in Table 1b; and wherein n is an integer of 1 to 3. In some embodiments, n is 1. In some embodiments, n is 2. In some embodiments, n is 3. In some embodiments, L is a linker in Table 4 (e.g., a SMCC or MSPT linker in Table 4).

In some embodiments, provided herein are SARM1 RNAi agents comprising Formula (I): (R-L)ₙ-P, wherein R is a double stranded RNA (dsRNA) comprising a sense stand and an antisense strand, and the dsRNA is any dsRNA in Table 5 or 7 (e.g., dsRNA No. 1); wherein L is a linker, or optionally absent; wherein P is a protein comprising one monovalent human TfR binding domain; and wherein n is an integer of 1 to 3. In some embodiments, n is 1. In some embodiments, n is 2. In some embodiments, n is 3. In some embodiments, L is a linker in Table 4 (e.g., a SMCC or MSPT linker in Table 4).

In some embodiments, provided herein are SARM1 RNAi agents comprising Formula (I): (R-L)ₙ-P, wherein R is a double stranded RNA (dsRNA) comprising a sense stand and an antisense strand, and the dsRNA is any dsRNA in Table 5 or 7 (e.g., dsRNA No. 1); wherein L is a linker, or optionally absent; wherein P is a protein comprising one monovalent human TfR binding domain, and P is selected from TBP1, TBP2, TBP3, TBP4, or TBP5 in Table 1b; and wherein n is an integer of 1 to 3. In some embodiments, n is 1. In some embodiments, n is 2. In some embodiments, n is 3. In some embodiments, L is a linker in Table 4 (e.g., a SMCC or MSPT linker in Table 4).

### Human TfR Binding Proteins

The SARM1 RNAi agents described herein comprise a protein comprising one monovalent human TfR binding domain (“human TfR binding protein”). Human TfR binding protein of the SARM1 RNAi agents can bind TfR on BBB and transport the dsRNA into the CNS.

Exemplary sequences of human TfR binding domains and proteins are provided in Table 1a and 1b. In some embodiments, the monovalent human TfR binding domain comprises a heavy chain variable region (VH) and a light chain variable region (VL), and the VH comprises heavy chain complementarity determining regions HCDR1, HCDR2, and HCDR3, and the VL comprises light chain complementarity determining regions LCDR1, LCDR2, and LCDR3. In some embodiments, HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6. In some embodiments, VH comprises SEQ ID NO: 7, and VL comprises SEQ ID NO: 8. In some embodiments, VH comprises a sequence having at least 95% sequence identity to SEQ ID NO: 7, and VL comprises a sequence having at least 95% sequence identity to SEQ ID NO: 8.

TABLE 1a
Exemplary sequences of human TfR binding domains and proteins

| Region | Sequence | SEQ ID NO |
|---|---|---|
| HCDR1 | SYSMN | 1 |
| (KABAT) | | |
| HCDR2 | SISSSSSYIYYADSVKG | 2 |
| (KABAT) | | |
| HCDR3 | RHGYSNSDAFDN | 3 |
| (KABAT) | | |
| LCDR1 | RASQGISHYLV | 4 |
| (KABAT) | | |
| LCDR2 | AASSLQS | 5 |
| (KABAT) | | |
| LCDR3 | LQHNSYPWT | 6 |
| (KABAT) | | |
| VH | EVQLVESGGGLVKPGGSLRLSCVASGFTFSSYSMN | 7 |
| | WVRQAPGKGLEWVSSISSSSSYIYYADSVKGRFTIS | |
| | RDNAKNSLYLQMNSLRAEDTAVYYCARRHGYSN | |
| | SDAFDNWGQGTLVTVSS | |
| VL | DIQMTQSPSAMSASVGDRVTITCRASQGISHYLVW | 8 |
| | FQQKPGKVPKRLIYAASSLQSGVPSRFSGSGSGTEF | |
| | TLTISSLQPEDFATYYCLQHNSYPWTFGQGTKVEI | |
| | K | |
| Fab HC | EVQLVESGGGLVKPGGSLRLSCVASGFTFSSYSMN | 9 |
| | WVRQAPGKGLEWVSSISSSSSYIYYADSVKGRFTIS | |
| | RDNAKNSLYLQMNSLRAEDTAVYYCARRHGYSN | |
| | SDAFDNWGQGTLVTVSSASTKGPSVFPLAPSSKST | |
| | SGGTAALGCLVKDYFPEPVTVSWNSGALTSGVHT | |
| | FPAVLQSSGLYSLSSVVTVPSSSLGTQTYICNVNHK | |
| | PSNTKVDKRVEPKC | |
| Fab LC/Fab- | DIQMTQSPSAMSASVGDRVTITCRASQGISHYLVW | 10 |
| VHH LC | FQQKPGKVPKRLIYAASSLQSGVPSRFSGSGSGTEF | |
| | TLTISSLQPEDFATYYCLQHNSYPWTFGQGTKVEI | |
| | KRTVAAPSVFIFPPSDEQLKSGTASVVCLLNNFYPR | |
| | EAKVQWKVDNALQSGNSQESVTEQDSKDSTYSLS | |
| | STLTLSKADYEKHKVYACEVTHQGLSSPVTKSFNR | |
| | GEC | |
| Fab-VHH HC | EVQLVESGGGLVKPGGSLRLSCVASGFTFSSYSMN | 11 |
| | WVRQAPGKGLEWVSSISSSSSYIYYADSVKGRFTIS | |
| | RDNAKNSLYLQMNSLRAEDTAVYYCARRHGYSN | |
| | SDAFDNWGQGTLVTVSSASTKGPCVFPLAPSSKST | |
| | SGGTAALGCLVKDYFPEPVTVSWNSGALTSGVHT | |
| | FPAVLQSSGLYSLSSVVTVPSSSLGTQTYICNVNHK | |
| | PSNTKVDKRVEPKCDKTHTGGGGQGGGGQGGGG | |
| | QGGGGQGGGGQEVQLLESGGGLVQPGGSLRLSCA | |
| | ASGRYIDETAVAWFRQAPGKGREFVAGIGGGVDI | |
| | TYYADSVKGRFTISRDNSKNTLYLQMNSLRPEDTA | |
| | VYYCGARPGRPLITSKVADLYPYWGQGTLVTVSS | |
| | PP | |
| Fab-VHH LC | DIQMTQSPSAMSASVGDRVTITCRASQGISHYLVW | 12 |
| | FQQKPGKVPKRLIYAASSLQSGVPSRFSGSGSGTEF | |
| | TLTISSLQPEDFATYYCLQHNSYPWTFGQGTKVEI | |
| | KRTVAAPSVFIFPPSDEQLKSGTASVVCLLNNFYPR | |
| | EAKVQWKVDNALQCGNSQESVTEQDSKDSTYSLS | |
| | STLTLSKADYEKHKVYACEVTHQGLSSPVTKSFNR | |
| | GEC | |
| hIgG4 PAA HC | EVQLVESGGGLVKPGGSLRLSCVASGFTFSSYSMN | 13 |
| | WVRQAPGKGLEWVSSISSSSSYIYYADSVKGRFTIS | |
| | RDNAKNSLYLQMNSLRAEDTAVYYCARRHGYSN | |
| | SDAFDNWGQGTLVTVSSASTKGPXVFPLAPCSRST | |
| | SESTAALGCLVKDYFPEPVTVSWNSGALTSGVHTF | |
| | PAVLQSSGLYSLSSVVTVPSSSLGTKTYTCNVDHK | |
| | PSNTKVDKRVESKYGPPCPPCPAPEAAGGPSVFLF | |
| | PPKPKDTLMISRTPEVTCVVVDVSQEDPEVQFNW | |
| | YVDGVEVHNAKTKPREEQFNSTYRVVSVLTVLHQ | |
| | DWLNGKEYKCKVSNKGLPSSIEKTISKAKGQPREP | |
| | QVYTLPPSQEEMTKNQVSLTCLVKGFYPSDIAVE | |
| | WESNGQPENNYKTTPPVLDSDGSFFLYSRLTVDKS | |
| | RWQEGNVFSCSVMHEALHNHYTQKSLSLSLG, | |
| | wherein X is S or C. | |
| OAH1 (one arm | EVQLVESGGGLVKPGGSLRLSCVASGFTFSSYSMN | 14 |
| heteromab) HC1 | WVRQAPGKGLEWVSSISSSSSYIYYADSVKGRFTIS | |
| (A378C) | RDNAKNSLYLQMNSLRAEDTAVYYCARRHGYSN | |
| | SDAFDNWGQGTLVTVSSASTKGPSVFPLAPCSRST | |
| | SESTAALGCLVKDYFPEPVTVSWNSGALTSGVHTF | |
| | PAVLQSSGLYSLSSVVTVPSSSLGTKTYTCNVDHK | |
| | PSNTKVDKRVESKYGPPCPPCPAPEAAGGPSVFLF | |
| | PPKPKDTLMISRTPEVTCVVVDVSQEDPEVQFNW | |
| | YVDGVEVHNAKTKPREEQFNSTYRVVSVLTVLHQ | |
| | DWLNGKEYKCKVSNKGLPSSIEKTISKAKGQPREP | |
| | QVSTLPPSQEEMTKNQVSLMCLVYGFYPSDIXVE | |
| | WESNGQPENNYKTTPPVLDSDGSFFLYSVLTVDKS | |
| | RWQEGNVFSCSVMHEALHNHYTQKSLSLSLG, | |
| | wherein X is A or C. | |
| OAH1 HC2 | ESKYGPPCPPCPAPEAAGGPSVFLFPPKPKDTLMIS | 15 |
| (A378C) | RTPEVTCVVVDVSQEDPEVQFNWYVDGVEVHNA | |
| | KTKPREEQFNSTYRVVSVLTVLHQDWLNGKEYKC | |
| | KVSNKGLPSSIEKTISKAKGQPREPQVYTLPPSQGD | |
| | MTKNQVQLTCLVKGFYPSDIXVEWESNGQPENNY | |
| | KTTPPVLDSDGSFFLASRLTVDKSRWQEGNVFSCS | |
| | VMHEALHNHYTQKSLSLSLG, wherein X is A or C. | |
| OAH LC | DIQMTQSPSAMSASVGDRVTITCRASQGISHYLVW | 10 |
| | FQQKPGKVPKRLIYAASSLQSGVPSRFSGSGSGTEF | |
| | TLTISSLQPEDFATYYCLQHNSYPWTFGQGTKVEI | |
| | KRTVAAPSVFIFPPSDEQLKSGTASVVCLLNNFYPR | |
| | EAKVQWKVDNALQSGNSQESVTEQDSKDSTYSLS | |
| | STLTLSKADYEKHKVYACEVTHQGLSSPVTKSFNR | |
| | GEC | |
| OAH2 HC1 | EVQLVESGGGLVKPGGSLRLSCVASGFTFSSYSMN | 16 |
| (S124C) | WVRQAPGKGLEWVSSISSSSSYIYYADSVKGRFTIS | |
| | RDNAKNSLYLQMNSLRAEDTAVYYCARRHGYSN | |
| | SDAFDNWGQGTLVTVSSASTKGPCVFPLAPCSRST | |
| | SESTAALGCLVKDYFPEPVTVSWNSGALTSGVHTF | |
| | PAVLQSSGLYSLSSVVTVPSSSLGTKTYTCNVDHK | |
| | PSNTKVDKRVESKYGPPCPPCPAPEAAGGPSVFLF | |
| | PPKPKDTLMISRTPEVTCVVVDVSQEDPEVQFNW | |
| | YVDGVEVHNAKTKPREEQFNSTYRVVSVLTVLHQ | |
| | DWLNGKEYKCKVSNKGLPSSIEKTISKAKGQPREP | |
| | QVSTLPPSQEEMTKNQVSLMCLVYGFYPSDIAVE | |
| | WESNGQPENNYKTTPPVLDSDGSFFLYSVLTVDKS | |
| | RWQEGNVFSCSVMHEALHNHYTQKSLSLSLG | |
| OAH2 HC2 | ESKYGPPCPPCPAPEAAGGPSVFLFPPKPKDTLMIS | 17 |
| (S124C) | RTPEVTCVVVDVSQEDPEVQFNWYVDGVEVHNA | |
| | KTKPREEQFNSTYRVVSVLTVLHQDWLNGKEYKC | |
| | KVSNKGLPSSIEKTISKAKGQPREPQVYTLPPSQGD | |
| | MTKNQVQLTCLVKGFYPSDIAVEWESNGQPENNY | |
| | KTTPPVLDSDGSFFLASRLTVDKSRWQEGNVFSCS | |
| | VMHEALHNHYTQKSLSLSLG | |
| Null Arm HC | QVQLVQSGAEVKKPGSSVKVSCKASGYTFSSYAIE | 18 |
| | WVRQAPGQGLEWMGGILPGSGTINYNEKFKGRVT | |
| | ITADKSTSTAYMELSSLRSEDTAVYYCARMSSNSD | |
| | QGFDLWGQGTLVTVSSASTKGPXVFPLAPCSRSTS | |
| | ESTAALGCLVKDYFPEPVTVSWNSGALTSGVHTFP | |
| | AVLQSSGLYSLSSVVTVPSSSLGTKTYTCNVDHKP | |
| | SNTKVDKRVESKYGPPCPPCPAPEAAGGPSVFLFP | |
| | PKPKDTLMISRTPEVTCVVVDVSQEDPEVQFNWY | |
| | VDGVEVHNAKTKPREEQFNSTYRVVSVLTVLHQD | |
| | WLNGKEYKCKVSNKGLPSSIEKTISKAKGQPREPQ | |
| | VYTLPPSQEEMTKNQVSLTCLVKGFYPSDIAVEWE | |
| | SNGQPENNYKTTPPVLDSDGSFLLYSKLTVDKSR | |
| | WQEGNVFSCSVMHEALHNHYTQKSLSLSLG, | |
| | wherein X is S or C. | |
| Null Arm LC | DIQMTQSPSSLSASVGDRVTITCKASQGISRFLSWF | 19 |
| | QQKPGKAPKSLIYAVSSLVDGVPSRFSGSGSGTDF | |
| | TLTISSLQPEDFATYYCVQYNSYPYGFGGGTKVEI | |
| | KRTVAAPSVFIFPPSDEQLKSGTASVVCLLNNFYPR | |
| | EAKVQWKVDNALQSGNSQESVTEQDSKDSTYSLS | |
| | STLTLSKADYEKHKVYACEVTHQGLSSPVTKSFNR | |
| | GEC | |

TABLE 1b
Exemplary sequences of human TfR binding proteins

| Human TfR | | | | |
|---|---|---|---|---|
| binding | | | | |
| protein (TBP) | HC1 | LC1 | HC2 | LC2 |
| TBP1 | SEQ ID NO: 9 | SEQ ID NO: 10 | N/A | N/A |
| (Fab) | | | | |
| TBP2 | SEQ ID NO: 11 | SEQ ID NO: 12 | N/A | N/A |
| (Fab-VHH) | | or 10 | | |
| TBP3 | SEQ ID NO: 13 | SEQ ID NO: 10 | SEQ ID NO: 18 | SEQ ID NO: 19 |
| (Heterodimeric | | | | |
| Ab) | | | | |
| TBP4 | SEQ ID NO: 14 | SEQ ID NO: 10 | SEQ ID NO: 15 | N/A* |
| (One Arm | | | | |
| Heteromab 1, | | | | |
| A378C) | | | | |
| TBP5 | SEQ ID NO: 16 | SEQ ID NO: 10 | SEQ ID NO: 17 | N/A* |
| (One Arm | | | | |
| Heteromab 2, | | | | |
| S124C) | | | | |

In some embodiments, the monovalent human TfR binding domain is an antibody fragment, e.g., Fab, scFv, Fv, or scFab (single chain Fab). In some embodiments, the monovalent human TfR binding domain is Fab. In some embodiments, the human TfR binding domain further comprises a heavy chain constant region and/or a light chain constant region.

In some embodiments, the human TfR binding protein further comprises a half-life extender, e.g., an immunoglobulin Fc region or a VHH that binds human serum albumin (HSA).

In some embodiments, the human TfR binding protein further comprises an immunoglobulin Fc region, e.g., a modified human IgG4 Fc region, or a modified human IgG1 Fc region. In some embodiments, the human TfR binding protein further comprises a modified human IgG4 Fc region comprising proline at residue 228, and alanine at residues 234 and 235 (all residues are numbered according to the EU Index numbering, also called hIgG4PAA Fc region). In some embodiments, the human TfR binding protein further comprises a modified human IgG1 Fc region comprising alanine at residues 234, 235, and 329, serine at position 265, aspartic acid at position 436 (all residues are numbered according to the EU Index numbering, also called hIgG1 effector null or hIgG1EN Fc region).

In some embodiments, the human TfR binding protein further comprise a VHH that binds human HSA. In some embodiments, the VHH also binds mouse, rat, and/or cynomolgus monkey albumin. An exemplary VHH that binds human HSA is shown in Table 2. In some embodiments, such a VHH comprises CDR1 comprising SEQ ID NO: 20, CDR2 comprising SEQ ID NO: 21, and CDR3 comprising SEQ ID NO: 22. In some embodiments, such a VHH comprises SEQ ID NO: 23. In some embodiments, the VHH is linked to the TfR binding domain through a peptide linker, e.g., (GGGGQ) 4 (SEQ ID NO: 24). In some embodiments, the VHH is linked to the C-terminus of the TfR binding domain.

TABLE 2
Exemplary sequences of VHH that binds human serum albumin (HSA)

| Region | Sequence | SEQ ID NO |
|---|---|---|
| CDR1 | ETAVA | 20 |
| (KABAT) | | |
| CDR2 | GIGGGVDITYYADSVKG | 21 |
| (KABAT) | | |
| CDR3 | RPGRPLITSKVADLYPY | 22 |
| (KABAT) | | |
| VHH full | EVQLLESGGGLVQPGGSLRLSCAASGRYIDETAV | 23 |
| length | AWFRQAPGKGREFVAGIGGGVDITYYADSVKGR | |
| | FTISRDNSKNTLYLQMNSLRPEDTAVYYCGARPG | |
| | RPLITSKVADLYPYWGQGTLVTVSSPP | |
| Optional | GGGGQGGGGQGGGGQGGGGQ | 24 |
| linker | | |

In some embodiments, the human TfR binding protein is heterodimeric antibody that comprises a first arm comprising one monovalent human TfR binding domain and a second arm that is a null arm, e.g., an arm that does not bind any known human target (e.g., an isotype arm). Heterodimeric antibodies such as heteromab, orthomab or duobody have been described in WO2014150973, WO2016118742, WO2018118616, and WO2011131746. In some embodiments, the first arm comprises any monovalent human TfR binding domain described herein. In some embodiments, the second arm is a null arm that does not bind any known human target (e.g., an isotype arm) comprises the sequences in Table 1a. In some embodiments, the second arm comprises a heavy chain (HC) and a light chain (LC), wherein the HC comprises SEQ ID NO: 18, and the LC comprises SEQ ID NO: 19.

In some embodiments, the human TfR binding protein comprises heterodimeric mutations. In some embodiments, the human TfR binding protein comprises a modified Fc region comprising a first Fc CH3 domain comprising serine at residue 349, methionine at residue 366, tyrosine at residue 370, and valine at residue 409, and a second Fc CH3 domain comprising glycine at residue 356, aspartic acid at residue 357, glutamine at residue 364 and alanine at residue 407 (all residues are numbered according to the EU Index numbering). In some embodiments, the human TfR binding protein comprises a modified Fc region comprising a first Fc CH3 domain comprising leucine at residue 405, and a second Fc CH3 domain comprising arginine at residue 409 (all residues are numbered according to the EU Index numbering).

In some embodiments, the human TfR binding protein comprises one or more native cysteine residues, which can be used for conjugation. For example, in some embodiments, the human TfR binding protein comprises a native cysteine at position 220 of the light chain and/or a native cysteine at position 226 of the heavy chain, which can be used for conjugation (all residues according to the EU Index numbering).

In some embodiments, the human TfR binding protein comprises engineered cysteine residues for conjugation. The approach of including engineered cysteines as a means for conjugation has been described in WO 2018/232088. In some embodiments, the human TfR binding protein comprises a heavy chain comprising one or more cysteines at the following residues: 124, 157, 162, 262, 373, 375, 378, 397, 415 (all residues according to the EU Index numbering). In some embodiments, the human TfR binding protein comprises a light chain (e.g., a kappa light chain) comprising one or more cysteines at the following residues: 156, 171, 191, 193, 202, 208 (all residues according to the EU Index numbering). In some embodiments, the human TfR binding protein comprises a heavy chain constant region comprising cysteine at residue 124 (according to the EU Index numbering). In some embodiments, the human TfR binding protein comprises a light chain constant region comprising cysteine at residue 156 (according to the EU Index numbering). In some embodiments, the human TfR binding protein comprises an immunoglobulin Fc region comprising cysteine at residue 378 (according to the EU Index numbering).

In some embodiments, the human TfR binding protein is any one of the human TfR binding proteins in Table 1b, e.g., TBP1, TBP2, TBP3, TBP4, TBP5.

In some embodiments, the human TfR binding protein has a Fab format, e.g., TBP1. In some embodiments, the human TfR binding protein comprises one HC and one LC, and wherein the HC comprises SEQ ID NO: 9 and the LC comprises SEQ ID NO: 10.

In some embodiments, the human TfR binding protein has a Fab-VHH format, e.g., TBP2. In some embodiments, the human TfR binding proteins comprises one HC and one LC, wherein the HC comprises SEQ ID NO: 11 and the LC comprises SEQ ID NO: 12 or 10.

In some embodiments, the human TfR binding protein has a heterodimeric antibody format, e.g., TBP3. In some embodiments, the human TfR binding protein comprises two heavy chains HC1 and HC2 and two light chains LC1 and LC2, wherein HCl comprises SEQ ID NO: 13, LC1 comprises SEQ ID NO: 10, HC2 comprises SEQ ID NO: 18, and LC2 comprises SEQ ID NO: 19.

In some embodiments, the human TfR binding protein has a one arm heteromab format, e.g., TBP4 or TBP5. In some embodiments, the human TfR binding protein comprises two heavy chains HC1 and HC2 and one light chain LC1, wherein HCl comprises SEQ ID NO: 14, LC1 comprises SEQ ID NO: 10, HC2 comprises SEQ ID NO: 15. In some embodiments, provided herein are human TfR binding proteins comprise two heavy chains HC1 and HC2 and one light chain LC1, wherein HC1 comprises SEQ ID NO: 16, LC1 comprises SEQ ID NO: 10, HC2 comprises SEQ ID NO: 17.

The human TfR binding proteins described herein can be recombinantly produced in a host cell, for example, using an expression vector. For example, an expression vector may include a sequence that encodes one or more signal peptides that facilitate secretion of the polypeptide(s) from a host cell. Expression vectors containing a polynucleotide of interest (e.g., a polynucleotide encoding a heavy chain or light chain of the TfR binding proteins) may be transferred into a host cell by well-known methods. Additionally, expression vectors may contain one or more selection markers, e.g., tetracycline, neomycin, and dihydrofolate reductase, to aide in detection of host cells transformed with the desired polynucleotide sequences.

A host cell includes cells stably or transiently transfected, transformed, transduced or infected with one or more expression vectors expressing all or a portion of the TfR binding proteins described herein. According to some embodiments, a host cell may be stably or transiently transfected, transformed, transduced or infected with an expression vector expressing HC polypeptides and an expression vector expressing LC polypeptides of the TfR binding proteins described herein. In some embodiments, a host cell may be stably or transiently transfected, transformed, transduced or infected with an expression vector expressing HC and LC polypeptides of the TfR binding proteins described herein. The TfR binding proteins may be produced in mammalian cells such as CHO, NSO, HEK293 or COS cells according to techniques well known in the art.

Medium, into which the TfR binding proteins has been secreted, may be purified by conventional techniques, such as mixed-mode methods of ion-exchange and hydrophobic interaction chromatography. For example, the medium may be applied to and eluted from a Protein A or G column using conventional methods; mixed-mode methods of ion-exchange and hydrophobic interaction chromatography may also be used. Soluble aggregate and multimers may be effectively removed by common techniques, including size exclusion, hydrophobic interaction, ion exchange, or hydroxyapatite chromatography. Various methods of protein purification may be employed, and such methods are known in the art and described, for example, in Deutscher, Methods in Enzymology 182:83-89 (1990) and Scopes, Protein Purification: Principles and Practice, 3rd Edition, Springer, NY (1994).

### Mouse TfR Binding Proteins

Some SARM1 RNAi agents used in the Examples below comprise a protein comprising one monovalent mouse TfR binding domain (“mouse TfR binding proteins” or mTBP). Exemplary sequences of mouse TfR binding proteins are provided in Table 3. Such SARM1 RNAi agents comprising a mouse TfR binding protein can serve as surrogate molecules in mouse models for SARM1 RNAi agents comprising a human TfR binding protein.

TABLE 3
Exemplary sequences of mouse TfR binding protein (mTBP1)

| Region | Sequence | SEQ ID NO |
|---|---|---|
| HCDR1 | GSYWIC | 25 |
| (KABAT) | | |
| HCDR2 | CIYSTSGGRTYYASWVKG | 26 |
| (KABAT) | | |
| HCDR3 | GDDSISDAYFDL | 27 |
| (KABAT) | | |
| LCDR1 | QSSQSVYNNNRLA | 28 |
| (KABAT) | | |
| LCDR2 | DASTLAS | 29 |
| (KABAT) | | |
| LCDR3 | QGTYFSSGWSWA | 30 |
| (KABAT) | | |
| VH | QSLEESGGDLVKPEGSLTLTCTASGFSFSGSYWI | 31 |
| | CWVRQAPGKGLEWIGCIYSTSGGRTYYASWVK | |
| | GRFTISKTSSTTVTLQMTSLTAADTATYFCARG | |
| | DDSISDAYFDLWGPGTLVTVSS | |
| VL | ALDMTQTASPVSAAVGGTVTINCQSSQSVYNN | 32 |
| | NRLAWYQQKPGQPPKLLIYDASTLASGVPSRFK | |
| | GSGSGTQFTLTISGVQSDDSATYYCQGTYFSSG | |
| | WSWAFGGGTEVVVK | |
| HC1 | QSLEESGGDLVKPEGSLTLTCTASGFSFSGSYWI | 33 |
| | CWVRQAPGKGLEWIGCIYSTSGGRTYYASWVK | |
| | GRFTISKTSSTTVTLQMTSLTAADTATYFCARG | |
| | DDSISDAYFDLWGPGTLVTVSSASTKGPCVFPL | |
| | APCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPPCPA | |
| | PEAAGGPSVFLFPPKPKDTLMISRTPEVTCVVVD | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPP | |
| | VLDSDGSFFLYSRLTVDKSRWQEGNVFSCSVM | |
| | HEALHNHYTQKSLSLSLG | |
| LC1 | ALDMTQTASPVSAAVGGTVTINCQSSQSVYNN | 34 |
| | NRLAWYQQKPGQPPKLLIYDASTLASGVPSRFK | |
| | GSGSGTQFTLTISGVQSDDSATYYCQGTYFSSG | |
| | WSWAFGGGTEVVVKRTVAAPSVFIFPPSDEQLK | |
| | SGTASVVCLLNNFYPREAKVQWKVDNALQSGN | |
| | SQESVTEQDSKDSTYSLSSTLTLSKADYEKHKV | |
| | YACEVTHQGLSSPVTKSFNRGEC | |
| Null Arm HC | QVQLVQSGAEVKKPGSSVKVSCKASGYTESSY | 18 |
| (HC2) | AIEWVRQAPGQGLEWMGGILPGSGTINYNEKFK | |
| | GRVTITADKSTSTAYMELSSLRSEDTAVYYCAR | |
| | MSSNSDQGFDLWGQGTLVTVSSASTKGPXVFPL | |
| | APCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPPCPA | |
| | PEAAGGPSVFLFPPKPKDTLMISRTPEVTCVVVD | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPP | |
| | VLDSDGSFLLYSKLTVDKSRWQEGNVFSCSVM | |
| | HEALHNHYTQKSLSLSLG, wherein X is S or C. | |
| Null Arm LC | DIQMTQSPSSLSASVGDRVTITCKASQGISRFLS | 19 |
| (LC2) | WFQQKPGKAPKSLIYAVSSLVDGVPSRFSGSGS | |
| | GTDFTLTISSLQPEDFATYYCVQYNSYPYGFGG | |
| | GTKVEIKRTVAAPSVFIFPPSDEQLKSGTASVVC | |
| | LLNNFYPREAKVQWKVDNALQSGNSQESVTEQ | |
| | DSKDSTYSLSSTLTLSKADYEKHKVYACEVTHQ | |
| | GLSSPVTKSFNRGEC | |

### Linker

In some embodiments, the SARM1 RNAi agents described herein comprises a linker that links the human TfR binding protein to the dsRNA. In some embodiments, the linker is a Mal-Tet-TCO linker, SMCC linker, GDM linker, MSPT linker or OD linker (structures of these linkers shown in Table 4). In some embodiments, the linker is a SMCC linker. In some embodiments, the linker is a MSPT linker.

TABLE 4
Exemplary linker structures

| Linker | Structure** |
|---|---|
| 1 | SMCC linker 1* |
| | 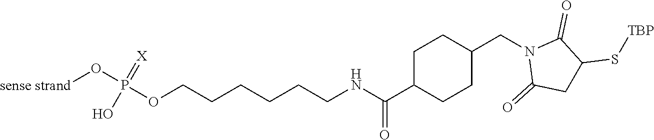 |
| 2 | SMCC linker 2 |
| | 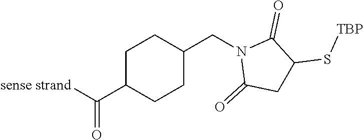 |
| 3 | Hydrolyzed ring open form of SMCC linker 1* |
| | 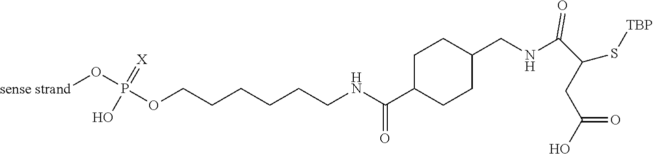 |
| 4 | Hydrolyzed ring open form of SMCC linker 2 |
| | 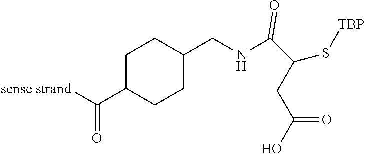 |
| 5 | Mal-Tet-TCO linker1* |
| | 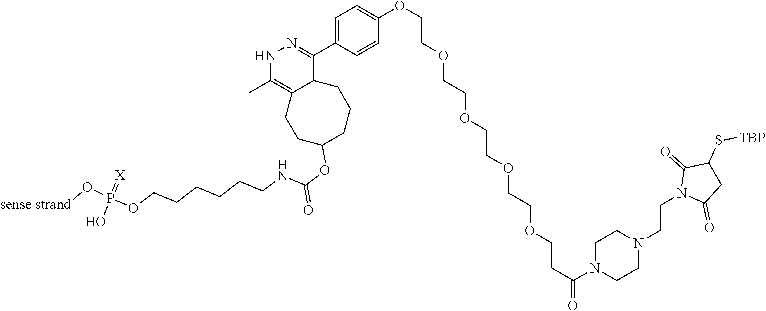 |
| 6 | Mal-Tet-TCO linker2 |
| | 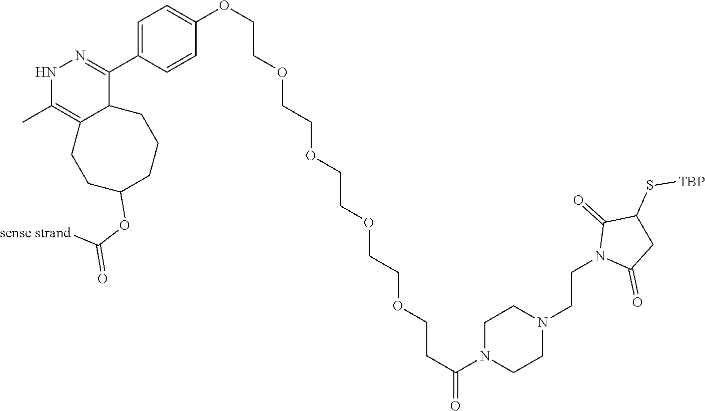 |
| 7 | GDM linker1* |
| | 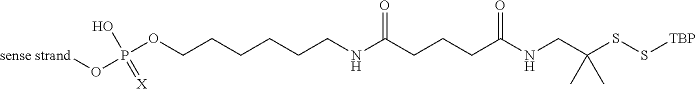 |
| 8 | GDM linker2 |
| | 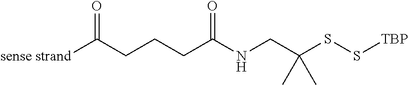 |
| 9 | 3′-OD linker* |
| | 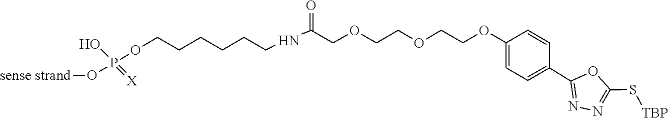 |
| 10 | 3′-MSPT linker* |
| | 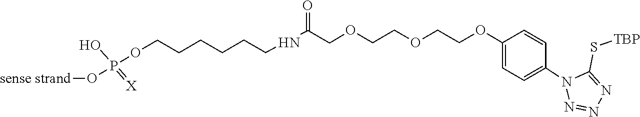 |
| 11 | 5′-OD linker |
| | 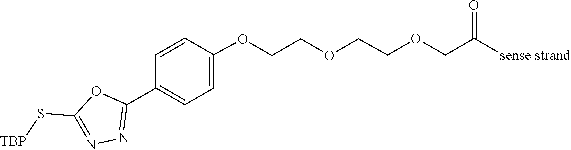 |
| 12 | 5′-MSPT linker |
| | 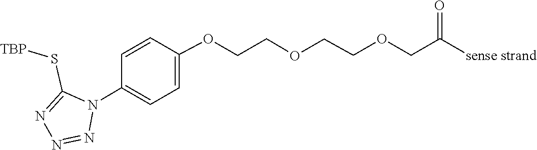 |

*Note-
X is O or S
**One skilled in the art will recognize that the sulfur connection to TBP may be considered as part of the TBP.

dsRNA

The SARM1 RNAi agents described herein comprise a double stranded RNA (dsRNA) comprising a sense stand and an antisense strand, and wherein the antisense strand is complementary to SARM1 mRNA. After the antisense strand of the dsRNA is incorporated into the RNA-induced silencing complex (RISC), the RISC can bind and degrade target SARM1 mRNA.

In some embodiments, the sense strand and the antisense strand of the dsRNA are each 15-30 nucleotides in length, e.g., 20-25 nucleotides in length. In some embodiments, the dsRNA has a sense strand of 21 nucleotides and an antisense strand of 23 nucleotides. In some embodiments, the sense strand and antisense strand of the dsRNA may have overhangs at either the 5′ end or the 3′ end (i.e., 5′ overhang or 3′ overhang). For example, the sense strand and the antisense strand may have 5′ or 3′ overhangs of 1 to 5 nucleotides or 1 to 3 nucleotides. In some embodiments, the antisense strand comprises a 3′ overhang of two nucleotides.

Exemplary unmodified sense strand and antisense strand sequences of dsRNA targeting human SARM1 mRNA are provided in Table 5.

TABLE 5
Unmodified Sequences of dsRNA targeting human SARM1 mRNA

| | | | | | Start position |
|---|---|---|---|---|---|
| | | | | | of antisense |
| | | | | | strand target |
| | | | | | region of |
| | | SEQ | | SEQ | human SARM1 |
| dsRNA | Sense Strand | ID | Antisense Strand | ID | transcript |
| No. | (5′ to 3′) | NO | (5′ to 3′) | NO | NM_015077.4* |
| 1 | GUUGCUCGACUCUAA | 35 | UAGCGGUUAGAGUCGAGC | 36 | 1333 |
| | CCGCUA | | AACGG | | |
| 2 | UUCGCCAACUAUUCU | 37 | UCACGUAGAAUAGUUGGC | 38 | 1763 |
| | ACGUGA | | GAAGG | | |
| 3 | ACCUUCGCCAACUAU | 39 | UGUAGAAUAGUUGGCGA | 40 | 1760 |
| | UCUACA | | AGGUCU | | |
| 4 | CCGCAAGAGGUUCUU | 41 | UCCCUAAAGAACCUCUUG | 42 | 1723 |
| | UAGGGA | | CGGGU | | |

In some embodiments, the sense strand and the antisense strand of the dsRNA comprise a pair of nucleic acid sequences selected from the group consisting of:
(a) the sense strand comprises SEQ ID NO: 35, and the antisense strand comprises SEQ ID NO: 36;
(b) the sense strand comprises SEQ ID NO: 37, and the antisense strand comprises SEQ ID NO: 38;
(c) the sense strand comprises SEQ ID NO: 39, and the antisense strand comprises SEQ ID NO: 40; and
(d) the sense strand comprises SEQ ID NO: 41, and the antisense strand comprises SEQ ID NO: 42;
   wherein optionally one or more nucleotides of the sense strand and the antisense strand are independently modified nucleotides, and wherein optionally one or more internucleotide linkages of the sense strand and the antisense strand are modified internucleotide linkages. In some embodiments, the sense strand comprises SEQ ID NO: 35, and the antisense strand comprises SEQ ID NO: 36. In some embodiments, the sense strand comprises SEQ ID NO: 37, and the antisense strand comprises SEQ ID NO: 38. In some embodiments, the sense strand comprises SEQ ID NO: 39, and the antisense strand comprises SEQ ID NO: 40. In some embodiments, the sense strand comprises SEQ ID NO: 41, and the antisense strand comprises SEQ ID NO: 42.

The dsRNA can include modifications. The modifications can be made to one or more nucleotides of the sense and/or antisense strand or to the internucleotide linkages, which are the bonds between two nucleotides in the sense or antisense strand. For example, some 2′-modifications of ribose or deoxyribose can increase RNA or DNA stability and half-life. Such 2′-modifications can be 2′-fluoro, 2′-O-methyl (i.e., 2′-methoxy), or 2′-O-alkyl (e.g., 2′-O—C₁₆ alkyl).

In some embodiments, one or more nucleotides of the sense strand and/or the antisense strand are independently modified nucleotides, which means the sense strand and the antisense strand can have different modified nucleotides. In some embodiments, each nucleotide of the sense strand is a modified nucleotide. In some embodiments, at least one nucleotide of the sense strand is an unmodified RNA nucleotide. In some embodiments, each nucleotide of the antisense strand is a modified nucleotide. In some embodiments, the modified nucleotide is a 2′-fluoro modified nucleotide, 2′-O-methyl modified nucleotide, 2′ deoxy nucleotide (DNA), or 2′-O-alkyl (e.g., 2′-O—C₁₆ alkyl) modified nucleotide. In some embodiments, each nucleotide of the sense strand and the antisense strand is independently a modified nucleotide, e.g., a 2′-fluoro modified nucleotide, 2′-O-methyl modified nucleotide, 2′ deoxy nucleotide (DNA), or 2′-O-alkyl (e.g., 2′-O—C₁₆ alkyl) modified nucleotide. In some embodiments, at least one nucleotide of the sense strand is 2′ deoxy nucleotide (DNA).

In some embodiments, the sense strand has four 2′-fluoro modified nucleotides, e.g., at positions 7, 9, 10, 11 from the 5′ end of the sense strand. In some embodiments, at least one nucleotide of the sense strand is an unmodified RNA nucleotide. In some embodiments, at least one nucleotide of the sense strand is 2′ deoxy nucleotide (DNA). In some embodiments, the other nucleotides of the sense strand are 2′-O-methyl modified nucleotides.

In some embodiments, the antisense strand has four 2′-fluoro modified nucleotides, e.g., at positions 2, 6, 14, 16 from the 5′ end of the antisense strand. In some embodiments, the other nucleotides of the antisense strand are 2′-O-methyl modified nucleotides.

In some embodiments, the sense strand has three 2′-fluoro modified nucleotides, e.g., at positions 9, 10, 11 from the 5′ end of the sense strand. In some embodiments, at least one nucleotide of the sense strand is an unmodified RNA nucleotide. In some embodiments, at least one nucleotide of the sense strand is 2′ deoxy nucleotide (DNA). In some embodiments, the other nucleotides of the sense strand are 2′-O-methyl modified nucleotides.

In some embodiments, the antisense strand has five 2′-fluoro modified nucleotides, e.g., at positions 2, 5, 7, 14, 16 from the 5′ end of the antisense strand. In some embodiments, the antisense strand has five 2′-fluoro modified nucleotides, e.g., at positions 2, 5, 8, 14, 16 from the 5′ end of the antisense strand. In some embodiments, the antisense strand has five 2′-fluoro modified nucleotides, e.g., at positions 2, 3, 7, 14, 16 from the 5′ end of the antisense strand. In some embodiments, the antisense strand has three 2′-fluoro modified nucleotides, e.g., at positions 2, 14, 16 from the 5′ end of the antisense strand. In some embodiments, the other nucleotides of the antisense strand are 2′-O-methyl modified nucleotides.

In some embodiments, the 5′ end of the antisense strand has a phosphate analog, e.g., 5′-vinylphosphonate (5′-VP).

In some embodiments, the sense strand or the antisense strand comprises an abasic moiety or inverted abasic moiety, e.g., a moiety shown in Table 6.

TABLE 6
Abasic or inverted abasic (iAb) moieties

| | Structure |
|---|---|
| 1 (abasic) | 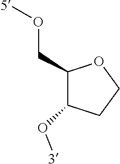 |
| 2 (iAb) | 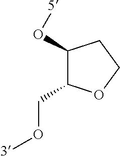 |

″5′″ and ″3′″ indicate the 5′ to 3′ direction of the sequences.

In some embodiments, the sense strand and the antisense strand have one or more modified internucleotide linkages. In some embodiments, the modified internucleotide linkage is phosphorothioate linkage. In some embodiments, the sense strand has four or five phosphorothioate linkages. In some embodiments, the antisense strand has four or five phosphorothioate linkages. In some embodiments, the sense strand and the antisense strand each has four or five phosphorothioate linkages. In some embodiments, the sense strand has four phosphorothioate linkages and the antisense strand has five phosphorothioate linkages.

Exemplary modified sense strand and antisense strand sequences of dsRNA targeting human SARM1 mRNA are provided in Table 7.

In some embodiments, the dsRNA comprises a sense strand that comprises a sequence that has 1, 2, or 3 differences from a sense stand sequence in Table 7. In some embodiments, the dsRNA comprises an antisense strand that comprises a sequence that has 1, 2, or 3 differences from an antisense stand sequence in Table 7.

TABLE 7
Modified Sequences of dsRNA targeting human SARM1 mRNA

| | | | SEQ |
|---|---|---|---|
| dsRNA | | | ID |
| No. | Strand | Sequence from 5′ to 3′ end | NO |
| 5 | S | mG*mU*mUmGmCmUmCmGfAfCfUmCmUmAmAmCmCmGmC*mU*mA | 43 |
| | AS | mU*fA*mGmCfGmGmUfUmAmGmAmGmUfCmGfAmGmCmAmAmC*mG*mG | 44 |
| 6 | S | mU*mU*mCmGmCmCmAmAfCfUfAmUmUmCmUmAmCmGmU*mG*mA | 45 |
| | AS | mU*fC*mAmCfGmUmAfGmAmAmUmAmGfUmUfGmGmCmGmAmA*mG*mG | 46 |
| 7 | S | mA*mC*mCmUmUmCmGmCfCfAfAmCmUmAmUmUmCmUmA*mC*mA | 47 |
| | AS | mU*fG*mUmAfGmAmAfUmAmGmUmUmGfGmCfGmAmAmGmGmU*mC*mU | 48 |
| 8 | S | mC*mC*mGmCmAmAmGmAfGfGfUmUmCmUmUmUmAmGmG*mG*mA | 49 |
| | AS | mU*fC*mCmCfUmAmAfAmGmAmAmCmCfUmCfUmUmGmCmGmG*mG*mU | 50 |
| 9 | S | mU*mU*mCmGmCmCmAmAfCfUfAmUmUmCmUmAmCmGmU*mG*mA | 45 |
| | AS | mU*fC*mAmCfGmUfAmGmAmAmUmAmGfUmUfGmGmCmGmAmA*mG*mG | 51 |
| 10 | S | mU*mU*mCmGmCmCmAmAfCfUfAmUmUmCmUmAmCmGmU*mG*mA | 45 |
| | AS | mU*fC*fAmCmGmUfAmGmAmAmUmAmGfUmUfGmGmCmGmAmA*mG*mG | 52 |
| 11 | S | mU*mU*mCmGmCmCmAmAfCfUfAmUmUmCmUmAmCmGmU*mG*mA | 45 |
| | AS | mU*fC*mAmCmGmUmAmGmAmAmUmAmGfUmUmGmGmCmGmAmA*mG*mG | 53 |
| 12 | S | mG*mU*mUmGmCmUmCmGfAfCfUmCmUmAmAmCmCmGmC*mU*mA | 43 |
| | AS | mU*fA*mGmCfGmGfUmUmAmGmAmGmUfCmGfAmGmCmAmAmC*mG*mG | 54 |
| 13 | S | mG*mU*mUmGmCmUmCmGfAfCfUmCmUmAmAmCmCmGmC*mU*mA | 43 |
| | AS | mU*fA*fGmCmGmGfUmUmAmGmAmGmUfCmGfAmGmCmAmAmC*mG*mG | 55 |
| 14 | S | mG*mU*mUmGmCmUmCmGfAfCfUmCmUmAmAmCmCmGmC*mU*mA | 43 |
| | AS | mU*fA*mGmCmGmGmUmUmAmGmAmGmUfCmGmAmGmCmAmAmC*mG*mG | 56 |
| 15 | S | mC*mC*mGmCmAmAmGmAfGfGfUmUmCmUmUmUmAmGmG*mG*mA | 49 |
| | AS | mU*fC*mCmCfUmAfAmAmGmAmAmCmCfUmCfUmUmGmCmGmG*mG*mU | 57 |
| 16 | S | mC*mC*mGmCmAmAmGmAfGfGfUmUmCmUmUmUmAmGmG*mG*mA | 49 |
| | AS | mU*fC*fCmCmUmAfAmAmGmAmAmCmCfUmCfUmUmGmCmGmG*mG*mU | 58 |
| 17 | S | mC*mC*mGmCmAmAmGmAfGfGfUmUmCmUmUmUmAmGmG*mG*mA | 49 |
| | AS | mU*fC*mCmCmUmAmAmAmGmAmAmCmCfUmCmUmUmGmCmGmG*mG*mU | 59 |
| 18 | S | mG*mU*mUmGmCmUfCmGfAfCfUmCmUmAmAmCmCmGmC*mU*mA | 43 |
| | AS | mU*fA*mGmCmGfGmUmUmAmGmAmGmUfCmGfAmGmCmAmAmC*mG*mG | 60 |
| 19 | S | mU*mU*mCmGmCmCfAmAfCfUfAmUmUmCmUmAmCmGmU*mG*mA | 45 |
| | AS | mU*fC*mAmCmGfUmAmGmAmAmUmAmGfUmUfGmGmCmGmAmA*mG*mG | 61 |
| 20 | S | mA*mC*mCmUmUmCfGmCfCfAfAmCmUmAmUmUmCmUmA*mC*mA | 47 |
| | AS | mU*fG*mUmAmGfAmAmUmAmGmUmUmGfGmCfGmAmAmGmGmU*mC*mU | 62 |
| 21 | S | mC*mC*mGmCmAmAfGmAfGfGfUmUmCmUmUmUmAmGmG*mG*mA | 49 |
| | AS | mU*fC*mCmCmUfAmAmAmGmAmAmCmCfUmCfUmUmGmCmGmG*mG*mU | 63 |

Abbreviations-“m” indicates 2′-OMe; “f” indicates 2′-fluoro; “*” indicates phosphorothioate linkage; “S” means the sense strand; “AS” means the antisense strand; unless otherwise noted, the 5′ position of the AS can include 5′-phosphate or 5′-vinylphosphonate (VP).

In some embodiments, the sense strand and the antisense strand of the dsRNA comprise a pair of nucleic acid sequences selected from the group consisting of:
(a) the sense strand comprises SEQ ID NO: 43, and the antisense strand comprises SEQ ID NO: 44, 54, 55, 56, or 60;
(b) the sense strand comprises SEQ ID NO: 45, and the antisense strand comprises SEQ ID NO: 46, 51, 52, 53, or 61;
(c) the sense strand comprises SEQ ID NO: 47, and the antisense strand comprises SEQ ID NO: 48 or 62; and
(d) the sense strand comprises SEQ ID NO: 49, and the antisense strand comprises SEQ ID NO: 50, 57, 58, 59, or 63.

In some embodiments, the sense strand and the antisense strand of the dsRNA have a pair of nucleic acid sequences selected from the group consisting of:
(a) the sense strand consists of SEQ ID NO: 43, and the antisense strand consists of SEQ ID NO: 44, 54, 55, 56, or 60;
(b) the sense strand consists of SEQ ID NO: 45, and the antisense strand consists of SEQ ID NO: 46, 51, 52, 53, or 61;
(c) the sense strand consists of SEQ ID NO: 47, and the antisense strand consists of SEQ ID NO: 48 or 62; and
(d) the sense strand consists of SEQ ID NO: 49, and the antisense strand consists of SEQ ID NO: 50, 57, 58, 59, or 63.

The sense strand and antisense strand of dsRNA can be synthesized using any nucleic acid polymerization methods known in the art, for example, solid-phase synthesis by employing phosphoramidite chemistry methodology (e.g., Current Protocols in Nucleic Acid Chemistry, Beaucage, S. L. et al. (Edrs.), John Wiley & Sons, Inc., New York, NY, USA), H-phosphonate, phosphortriester chemistry, or enzymatic synthesis. Automated commercial synthesizers can be used, for example, MerMade™ 12 from LGC Biosearch Technologies, or other synthesizers from BioAutomation or Applied Biosystems. Phosphorothioate linkages can be introduced using a sulfurizing reagent such as phenylacetyl disulfide or DDTT (((dimethylaminomethylidene) amino)-3H-1,2,4-dithiazaoline-3-thione). It is well known to use similar techniques and commercially available modified amidites and controlled-pore glass (CPG) products to synthesize modified oligonucleotides or conjugated oligonucleotides.

Purification methods can be used to exclude the unwanted impurities from the final oligonucleotide product. Commonly used purification techniques for single stranded oligonucleotides include reverse-phase ion pair high performance liquid chromatography (RP-IP-HPLC), capillary gel electrophoresis (CGE), anion exchange HPLC (AX-HPLC), and size exclusion chromatography (SEC). After purification, oligonucleotides can be analyzed by mass spectrometry and quantified by spectrophotometry at a wavelength of 260 nm. The sense strand and antisense strand can then be annealed to form a dsRNA.

The RNAi agent described herein can be made by a variety of procedures known to one of ordinary skill in the art, some of which are illustrated in the preparations and examples below, e.g., in Examples 1-3. One of ordinary skill in the art recognizes that the specific synthetic steps for each of the routes described may be combined in different ways, or in conjunction with steps from different schemes, to prepare the RNAi agent. The product of each step can be recovered by conventional methods well known in the art, including extraction, evaporation, precipitation, chromatography, filtration, trituration, and crystallization. The reagents and starting materials are readily available to one of ordinary skill in the art.

In some embodiments, the TfR binding protein with native or engineered cysteines described herein can be first treated with a reducing agent, e.g., DTT, and then re-oxidized with an oxidizing agent, e.g., DHAA. The resulting oxidized TfR binding protein is then incubated with a linker functionalized dsRNA, e.g., linker-dsRNA, to produce the conjugated RNAi agent.

### Pharmaceutical Composition

In another aspect, provided herein are pharmaceutical compositions comprising any of the SARM1 RNAi agents described herein and a pharmaceutically acceptable carrier. Such pharmaceutical compositions can also comprise one or more pharmaceutically acceptable excipient, diluent, or carrier. Pharmaceutical compositions can be prepared by methods well known in the art (e.g., Remington: The Science and Practice of Pharmacy, 23rd edition (2020), A. Loyd et al., Academic Press).

### Method of Treatment and Therapeutic Use

In another aspect, provided herein are methods of reducing axon degeneration in a patient in need thereof, and such method comprises administering to the patient an effective amount of a SARM1 RNAi agent or a pharmaceutical composition described herein.

In another aspect, provided herein are methods of treating a SARM1-mediated neurological disease in a patient in need thereof, and such method comprises administering to the patient an effective amount of the SARM1 RNAi agent or a pharmaceutical composition described herein. Exemplary SARM1-mediated neurological disease includes, but are not limited to, amyotrophic lateral sclerosis (ALS, or Lou Gehrig's disease), Alzheimer's disease, Parkinson's disease, multiple sclerosis (MS), Huntington's disease (HD), senile dementia, Pick's disease, Gaucher's disease, Hurler syndrome, progressive multifocal leukoencephalopathy, Alexander's disease, congenital hypomyelination, encephalomyelitis, acute disseminated encephalomyelitis, central pontine myelinolysis, osmotic hyponatremia, Tay-Sachs disease, motor neuron disease, ataxia, spinal muscular atrophy (SMA), Niemann-Pick disease, acute hemorrhagic leukoencephalitis, trigeminal neuralgia, Bell's palsy, cerebral ischemia, multiple system atrophy, Pelizaeus Merzbacher disease, periventricular leukomalacia, a hereditary ataxia, noise-induced hearing loss, congenital hearing loss, age-related hearing loss, Creutzfeldt-Jakob disease, transmissible spongiform encephalopathy, Lewy Body Dementia, frontotemporal dementia, tauopathy, synucleinopathy, amyloidosis, diabetic neuropathy, globoid cell leukodystrophy (Krabbe's disease), Bassen-Komzweig syndrome, transverse myelitis, motor neuron disease, spinocerebellar ataxia, pre-eclampsia, hereditary spastic paraplegias, spastic paraparesis, familial spastic paraplegia, French settlement disease, Strumpell-Lorrain disease, non-alcoholic steatohepatitis (NASH), adrenomyeloneuropathy, progressive supra nuclear palsy (PSP), Friedrich's ataxia, spinal cord injury, acute optic neuropathy (AON), a genetic or idiopathic retinal condition, Leber congenital amaurosis (LCA), Leber hereditary optic neuropathy (LHON), primary open-angle glaucoma (POAG), acute angle-closure glaucoma (AACG), autosomal dominant optic atrophy, retinal ganglion degeneration, retinitis pigmentosa, an outer retinal neuropathy, optic nerve neuritis, optic nerve degeneration associated with multiple sclerosis, Kjer's optic neuropathy, ischemic optic neuropathy, chemotherapy-induced peripheral neuropathy, neuromyelitis optica, Charcot Marie Tooth disease, deficiency in vitamin B12, deficiency in folic acid (vitamin B9), isolated vitamin E deficiency syndrome, non-arteritic anterior ischemic optic neuropathy, exposure to ethambutol, exposure to cyanide, traumatic brain injury (TBI), spinal cord injury, traumatic axonal injury or chronic traumatic encephalopathy (CTE). In some embodiments, the SARM1-mediated neurological disease is amyotrophic lateral sclerosis, multiple sclerosis, chemotherapy-induced peripheral neuropathy (CIPN), diabetic peripheral neuropathy (DPN), tauopathy, or Charcot Marie Tooth disease.

In some embodiments, SARM1-mediated neurological disease is a CNS neurodegenerative disease. In some embodiments, the SARM1-mediated neurological disease is amyotrophic lateral sclerosis (ALS, or Lou Gehrig's disease), Alzheimer's disease, Parkinson's disease, multiple sclerosis (MS), Huntington's disease (HD), Pick's disease, progressive multifocal leukoencephalopathy, Alexander's disease, congenital hypomyelination, encephalomyelitis, acute disseminated encephalomyelitis, central pontine myelinolysis, Tay-Sachs disease, spinal muscular atrophy (SMA), cerebral ischemia, Lewy Body Dementia, frontotemporal dementia, tauopathy, synucleinopathy, amyloidosis, diabetic neuropathy, globoid cell leukodystrophy (Krabbe's disease), Strumpell-Lorrain disease, adrenomyeloneuropathy, progressive supra nuclear palsy (PSP), Leber hereditary optic neuropathy (LHON), optic nerve neuritis, optic nerve degeneration associated with multiple sclerosis, Kjer's optic neuropathy, ischemic optic neuropathy, chemotherapy-induced peripheral neuropathy, neuromyelitis optica, traumatic brain injury (TBI), spinal cord injury, traumatic axonal injury or chronic traumatic encephalopathy (CTE).

In some embodiments, the SARM1-mediated neurological disease is amyotrophic lateral sclerosis.

The SARM1 RNAi agent or a pharmaceutical composition comprising SARM1 RNAi agent can be administered to the patient intravenously or subcutaneously.

SARM1 RNAi agent dosage regimens may be adjusted to provide the optimum desired response (e.g., a therapeutic response). For example, a single bolus may be administered, several divided doses may be administered over time, or the dose may be proportionally reduced or increased as indicated by the exigencies of the therapeutic situation.

Dosage values may vary with the type and severity of the condition to be alleviated. It is further understood that for any particular subject, specific dosage regimens should be adjusted over time according to the individual need and the professional judgment of the person administering or supervising the administration of the compositions.

In another aspect, provided herein are SARM1 RNAi agents or pharmaceutical compositions comprising a SARM1 RNAi agent for use in reducing SARM1 expression. Also provided herein are SARM1 RNAi agents or pharmaceutical compositions comprising a SARM1 RNAi agent for use in reducing axon degeneration. Also provided herein are SARM1 RNAi agents or the pharmaceutical composition comprising a SARM1 RNAi agent for use in a therapy. Also provided herein are SARM1 RNAi agents or pharmaceutical compositions comprising a SARM1 RNAi agent for use in the treatment of a SARM1-mediated neurological disease. Also provided herein are uses of SARM1 RNAi agents in the manufacture of a medicament for reducing axon degeneration. Also provided herein are uses of SARM1 RNAi agents in the manufacture of a medicament for the treatment of a SARM1-mediated neurological disease.

### Definitions

As used herein, the terms “a,” “an,” “the,” and similar terms used in the context of the present disclosure (especially in the context of the claims) are to be construed to cover both the singular and plural unless otherwise indicated herein or clearly contradicted by the context.

As used herein, the term “alkyl” means saturated linear or branched-chain monovalent hydrocarbon radical, containing the indicated number of carbon atoms. For example, “C₁-C₂₀ alkyl” means a radical having 1-20 carbon atoms in a linear or branched arrangement.

The term “antibody,” as used herein, refers to a molecule that binds an antigen. Embodiments of an antibody include a monoclonal antibody, polyclonal antibody, human antibody, humanized antibody, chimeric antibody, heterodimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4).

An immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212).

Embodiments of the present disclosure also include antibody fragments or antigen-binding fragments that, as used herein, comprise at least a portion of an antibody retaining the ability to specifically interact with an antigen or an epitope of the antigen, such as Fab, Fab′, F(ab′)2, Fv fragments, scFv antibody fragments, scFab, disulfide-linked Fvs (sdFv), a Fd fragment.

The term “antigen binding domain”, as used herein, refers to a portion of an antibody or antibody fragment that binds an antigen or an epitope of the antigen. For example, “TfR binding domain” refers to a portion of an antibody or antibody fragment that binds TfR or an epitope of TfR.

The term “heterodimeric antibody”, as used herein, refers to an antibody that comprises two distinct antigen-binding domains.

As used herein, “antisense strand” means a single-stranded oligonucleotide that is complementary to a region of a target sequence. Likewise, and as used herein, “sense strand” means a single-stranded oligonucleotide that is complementary to a region of an antisense strand.

The terms “bind” and “binds” as used herein are intended to mean, unless indicated otherwise, the ability of a protein or molecule to form a chemical bond or attractive interaction with another protein or molecule, which results in proximity of the two proteins or molecules as determined by common methods known in the art.

As used herein, “complementary” means a structural relationship between two nucleotides (e.g., on two opposing nucleic acids or on opposing regions of a single nucleic acid strand, e.g., a hairpin) that permits the two nucleotides to form base pairs with one another. For example, a purine nucleotide of one nucleic acid that is complementary to a pyrimidine nucleotide of an opposing nucleic acid may base pair together by forming hydrogen bonds with one another. Complementary nucleotides can base pair in the Watson-Crick manner or in any other manner that allows for the formation of stable duplexes. Likewise, two nucleic acids may have regions of multiple nucleotides that are complementary with each other to form regions of complementarity, as described herein.

As used herein, “duplex,” in reference to nucleic acids or oligonucleotides, means a structure formed through complementary base pairing of two antiparallel sequences of nucleotides (i.e., in opposite directions), whether formed by two separate nucleic acid strands or by a single, folded strand (e.g., via a hairpin).

An “effective amount” refers to an amount necessary (for periods of time and for the means of administration) to achieve the desired therapeutic result. An effective amount of a protein or conjugate may vary according to factors such as the disease state, age, sex, and weight of the individual, and the ability of the protein or conjugate to elicit a desired response in the individual. An effective amount is also one in which any toxic or detrimental effects of the protein or conjugate are outweighed by the therapeutically beneficial effects.

The term “Fc region” as used herein refers to a polypeptide comprising the CH2 and CH3 domains of a constant region of an immunoglobulin, e.g., IgG1, IgG2, IgG3, or IgG4. Optionally, the Fc region may include a portion of the hinge region or the entire hinge region of an immunoglobulin, e.g., IgG1, IgG2, IgG3, or IgG4. In some embodiments, the Fc region is a human IgG Fc region, e.g., a human IgG1 Fc region, human IgG2 Fc region, human IgG3 Fc region or human IgG4 Fc region. In some embodiments, the Fc region is a modified IgG Fc region with reduced or eliminated effector functions compared to the corresponding wild type IgG Fc region. The numbering of the residues in the Fc region is based on the EU index as described in Kabat (Kabat et al, Sequences of Proteins of Immunological Interest, 5th edition, Bethesda, MD: U.S. Dept. of Health and Human Services, Public Health Service, National Institutes of Health, 1991). The boundaries of the Fc region of an immunoglobulin heavy chain might vary, and the human IgG heavy chain Fc region is usually defined as the stretch from the N-terminus of the CH2 domain (e.g., the amino acid residue at position 231 according to the EU index numbering) to the C-terminus of the CH3 domain (or the C-terminus of the immunoglobulin).

The term “knockdown” or “expression knockdown” refers to reduced mRNA or protein expression of a gene after treatment of a reagent.

As used herein, “modified internucleotide linkage” means an internucleotide linkage having one or more chemical modifications when compared with a reference internucleotide linkage having a phosphodiester bond. A modified internucleotide linkage can be a non-naturally occurring linkage. In some embodiments, the modified internucleotide linkage is phosphorothioate linkage.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can have, for example, one or more chemical modification in its sugar, nucleobase, and/or phosphate group. Additionally, or alternatively, a modified nucleotide can have one or more chemical moieties conjugated to a corresponding reference nucleotide. In some embodiments, the modified nucleotide is a 2′-fluoro modified nucleotide, 2′-O-methyl modified nucleotide, 2′ deoxy nucleotide (DNA), or 2′-O-alkyl (e.g., 2′-O—C₁₆ alkyl) modified nucleotide. In some embodiments, the modified nucleotide has a phosphate analog, e.g., 5′-vinylphosphonate. In some embodiments, the modified nucleotide has an abasic moiety or inverted abasic moiety, e.g., a moiety shown in Table 6.

As used herein, “nucleotide” means an organic compound having a nucleoside (a nucleobase, e.g., adenine, cytosine, guanine, thymine, or uracil, and a pentose sugar, e.g., ribose or 2′-deoxyribose) linked to a phosphate group. A “nucleotide” can serve as a monomeric unit of nucleic acid polymers such as deoxyribonucleic acid (DNA) and ribonucleic acid (RNA).

As used herein, a “null arm” means an antibody arm that does not bind any known human target.

As used herein, “oligonucleotide” means a polymer of linked nucleotides, each of which can be modified or unmodified. An oligonucleotide is typically less than about 100 nucleotides in length.

As used herein, “overhang” means the unpaired nucleotide or nucleotides that protrude from the duplex structure of a double stranded oligonucleotide. An overhang may include one or more unpaired nucleotides extending from a duplex region at the 5′ terminus or 3′ terminus of a double stranded oligonucleotide. The overhang can be a 3′ or 5′ overhang on the antisense strand or sense strand of a double stranded oligonucleotide.

The term “patient”, as used herein, refers to a human patient.

As used herein, “phosphate analog” means a chemical moiety that mimics the electrostatic and/or steric properties of a phosphate group. In some embodiments, a phosphate analog is positioned at the 5′ end of an oligonucleotide in place of a 5′-phosphate, which is sometimes susceptible to enzymatic removal. A 5′ phosphate analog can include a phosphatase-resistant linkage. Examples of phosphate analogs include 5′ methylene phosphonate (5′-MP) and 5′-(E)-vinylphosphonate (5′-VP). In some embodiments, the phosphate analog is 5′-VP.

As used herein, “SARM1” (sterile alpha and TIR motif containing 1, also known as SARM; HsTIR; SAMD2; hSARM1; MyD88-5) refers to a human SARM1 mRNA transcript or a human SARM1 protein. The nucleotide sequence of human SARM1 mRNA can be found at NM_015077.4:

| (SEQ ID NO: 64) | |
|---|---|
| 1 | ATCTCCCAGC TCAGCCGAGC CCGTGCCCAG GCCACGCTTT GTTCCAGCCG CCGCCTCCTC |
| 61 | TACCCTACGG CGTCCGGAGC CATCCCTCGC CTGCTCGCTC TCTCCTTTCG CCCACTCCCT |
| 121 | GCATCTGGGC CTGCATCACC TTTGCCAACC GCTCCCCCGA TCCTGCCGAC ACTCCTCCCC |
| 181 | CAAACTTCTG ACCGGCACCC TTGCCTGGTA CCCTTCTCTC CATTCCTCCC CCTCCATCTT |
| 241 | CTTTCCCCGA CCCCTCTCGG GTCCCTCTTT TCCCAAAACC CGGGTCTCTC CGCGTGGCCC |
| 301 | CGCCTCCAGG CCGGGGATGT CCCCCGCGGC CCCGCGCCCA TGGTCCTGAC GCTGCTTCTC |
| 361 | TCCGCCTACA AGCTGTGTCG CTTCTTCGCC ATGTCGGGCC CACGGCCGGG CGCCGAGCGG |
| 421 | CTGGCGGTGC CTGGGCCAGA TGGGGGCGGT GGCACGGGCC CATGGTGGGC TGCGGGTGGC |
| 481 | CGCGGGCCCC GCGAAGTGTC GCCGGGGGCA GGCACCGAGG TGCAGGACGC CCTGGAGCGC |
| 541 | GCGCTGCCGG AGCTGCAGCA GGCCTTGTCC GCGCTGAAGC AGGCGGGCGG CGCGCGGGCC |
| 601 | GTGGGCGCCG GCCTGGCCGA GGTCTTCCAA CTGGTGGAGG AGGCCTGGCT GCTGCCGGCC |
| 661 | GTGGGCCGCG AGGTAGCCCA GGGTCTGTGC GACGCCATCC GCCTCGATGG CGGCCTCGAC |
| 721 | CTGCTGTTGC GGCTGCTGCA GGCGCCGGAG TTGGAGACGC GTGTGCAGGC CGCGCGCCTG |
| 781 | CTGGAGCAGA TCCTGGTGGC TGAGAACCGA GACCGCGTGG CGCGCATTGG GCTGGGCGTG |
| 841 | ATCCTGAACC TGGCGAAGGA ACGCGAACCC GTAGAGCTGG CGCGGAGCGT GGCAGGCATC |
| 901 | TTGGAGCACA TGTTCAAGCA TTCGGAGGAG ACATGCCAGA GGCTGGTGGC GGCCGGCGGC |
| 961 | CTGGACGCGG TGCTGTATTG GTGCCGCCGC ACGGACCCCG CGCTGCTGCG CCACTGCGCG |
| 1021 | CTGGCGCTGG GCAACTGCGC GCTGCACGGG GGCCAGGCGG TGCAGCGACG CATGGTAGAG |
| 1081 | AAGCGCGCAG CCGAGTGGCT CTTCCCGCTC GCCTTCTCCA AGGAGGACGA GCTGCTTCGG |
| 1141 | CTGCACGCCT GCCTCGCAGT AGCGGTGTTG GCGACTAACA AGGAGGTGGA GCGCGAGGTG |
| 1201 | GAGCGCTCGG GCACGCTGGC GCTCGTGGAG CCGCTTGTGG CCTCGCTGGA CCCTGGCCGC |
| 1261 | TTCGCCCGCT GTCTGGTGGA CGCCAGCGAC ACAAGCCAGG GCCGCGGGCC CGACGACCTG |
| 1321 | CAGCGCCTCG TGCCGTTGCT CGACTCTAAC CGCTTGGAGG CGCAGTGCAT CGGGGCTTTC |
| 1381 | TACCTCTGCG CCGAGGCTGC CATCAAGAGC CTGCAAGGCA AGACCAAGGT GTTCAGCGAC |
| 1441 | ATCGGCGCCA TCCAGAGCCT GAAACGCCTG GTTTCCTACT CTACCAATGG CACTAAGTCG |
| 1501 | GCGCTGGCCA AGCGCGCGCT GCGCCTGCTG GGCGAGGAGG TGCCACGGCC CATCCTGCCC |
| 1561 | TCCGTGCCCA GCTGGAAGGA GGCCGAGGTT CAGACGTGGC TGCAGCAGAT CGGTTTCTCC |
| 1621 | AAGTACTGCG AGAGCTTCCG GGAGCAGCAG GTGGATGGCG ACCTGCTTCT GCGGCTCACG |
| 1681 | GAGGAGGAAC TCCAGACCGA CCTGGGCATG AAATCGGGCA TCACCCGCAA GAGGTTCTTT |
| 1741 | AGGGAGCTCA CGGAGCTCAA GACCTTCGCC AACTATTCTA CGTGCGACCG CAGCAACCTG |
| 1801 | GCGGACTGGC TGGGCAGCCT GGACCCGCGC TTCCGCCAGT ACACCTACGG CCTGGTCAGC |
| 1861 | TGCGGCCTGG ACCGCTCCCT GCTGCACCGC GTGTCTGAGC AGCAGCTGCT GGAAGACTGC |
| 1921 | GGCATCCACC TGGGCGTGCA CCGCGCCCGC ATCCTCACGG CGGCCAGAGA AATGCTACAC |
| 1981 | TCCCCGCTGC CCTGTACTGG TGGCAAACCC AGTGGGGACA CTCCAGATGT CTTCATCAGC |
| 2041 | TACCGCCGGA ACTCAGGTTC CCAGCTGGCC AGTCTCCTGA AGGTGCACCT GCAGCTGCAT |
| 2101 | GGCTTCAGTG TCTTCATTGA TGTGGAGAAG CTGGAAGCAG GCAAGTTCGA GGACAAACTC |
| 2161 | ATCCAGAGTG TCATGGGTGC CCGCAACTTT GTGTTGGTGC TATCACCTGG AGCACTGGAC |
| 2221 | AAGTGCATGC AAGACCATGA CTGCAAGGAT TGGGTGCATA AGGAGATTGT GACTGCTTTA |
| 2281 | AGCTGCGGCA AGAACATTGT GCCCATCATT GATGGCTTCG AGTGGCCTGA GCCCCAGGTC |
| 2341 | CTGCCTGAGG ACATGCAGGC TGTGCTTACT TTCAACGGTA TCAAGTGGTC CCACGAATAC |
| 2401 | CAGGAGGCCA CCATTGAGAA GATCATCCGC TTCCTGCAGG GCCGCTCCTC CCGGGACTCA |
| 2461 | TCTGCAGGCT CTGACACCAG TTTGGAGGGT GCTGCACCCA TGGGTCCAAC CTAACCAGTC |
| 2521 | CCCAGTTCCC CAGCCCTGCT GTGACTTCCA TTTCCATCGT CCTTTCTGAA GGAACAGCTC |
| 2581 | CTGAAACCAG TCTCCCTGGG CTGAGACAAC CTGGGCTCTT CTTAGGAAAT GGCTCTCCCT |
| 2641 | CCCCCTGTCC CCCACCCTCA TGGCCCACCT CCAACCCACT TTCCTCAGTA TCTGGAGAGG |
| 2701 | GAAGGGAAGT CAGGCTTGGG CACGGGAGGT TAGAACTCCC CCAGGCCCTG CCATTGGGTT |
| 2761 | GTCTGTCTCC GTCATGGGGA GGGTCCCTGC TCAGTTCTGG AGACACTGGA GTTGGGGTGG |
| 2821 | GGGTGGTTCT GCATTCCCTT CTCCTGCTGA TAGCAGTCAG CTTGAGGAGG ATGACGGAAG |
| 2881 | GCAGCCTCAG ACAGGAATTA AGGCAATGCC CAGGCGGGCC TGGGCACTGT ATTCTGAGCA |
| 2941 | AGGGCCTGGG CCCAGGAGCC AGCCAGGGAT GAGTGCCATC ATGGCTCTCC ACTCAGACTG |
| 3001 | TGCCTGGCCC CTGCACTTAC AACTTCCTGC CGCTCTGTGG CCTTGCCCTG TAATCACTCA |
| 3061 | GTGCCCTTAG CTAGCCTGAC TAAGTCCCAG ATCCCCTACA GCTTCCTTCG GTGTGGTATC |
| 3121 | TTTTGCCACA TCCAGGGCGA GGGTTGAGGC AAACCAGCCC TCCCTCTGAC TTCCTTGTCA |
| 3181 | CTGCAGCCAG CTTTGCTGCA CTTGCTGGTG CACAGGAGCC TCCTGTTTGG GCCTGGGTCT |
| 3241 | GGGCATGGGG AGGCCGTGCC TCAAAGCCCA CCCTACCCCA TGCCTTGGTG CTGTGCCTCA |
| 3301 | GGCTCCTTCC TGGTCTGGCC CAGCTGGCTT CCCCAGCCCC TCAGCCATCC AGGGCTACCC |
| 3361 | ACTGCTTACT CAGGGACCAG GCAGCCCCCA TGGCAGTAAA AGCAGCCTAG ACAGAACCTG |
| 3421 | CAGCTCTGTG GAAAGAGGCA AAGTCCTGAA AAGGCAAAGG GTTGTCACTT AGGGCAGCTT |
| 3481 | CTCCAACTTT AACATGCATC CAAGTCACCT GGGAATGTTG TTAAAATCAG GAGATCTGGG |
| 3541 | GTGGGGCCTA GGACTCTGCA TTTCTTACAG ATTCCCAGGT GAGCTGATGC TGGTGGTTAA |
| 3601 | GGGTAGCAAA TCTCTAAAGC ACGAAGCCCT CACAAATCTT TGCCATTTCC CAAACACTCC |
| 3661 | GCTCCATGGT CTCCAGTCAT CAGAGCAACT CTACCTGGTA TTATCATCCC CATTTTACAG |
| 3721 | ATAATGACAC TGAGGCTCAG AAAGGTTGAG GATAAGCCCA CTTTCCTGTC ATTAGTGGCA |
| 3781 | GCCCCAGATC CAGACCTAGG CCTCCTGGCA CCCAGTCCAC TGGCAGTGGA ATTGCTTTCC |
| 3841 | TGAGAATCAT TCTGAGGCTG GGCTATTGCT TCTCCCTTGC TTCAAAGAAT CTAGCAGCGG |
| 3901 | GGGATAGGAT TTTGCAACAA AAAGCTGACC CAGAGGCCAT ACAGAGCAGG AATATCCCAT |
| 3961 | TGCCCCCTCC TCCACTGGGT TCAGAGGGTA AGAAAGCACC CTCCAATAAA CCCAGGCTCC |
| 4021 | AGGCCGTGGG GGCTGCTGAA GGCTCTTTCC CCGCAAGGGC CAGGTGTTGA CACCTTAAAG |
| 4081 | CTGGCTGCGC CCCCAGCCCC ACTCTTGGCT GTGCTGGCCA GGTGACTCCT AGTTCTTGGC |
| 4141 | CACATCATCA GAAAGTCAAA GGTCTCACTC CAGGTTTGGG GCTCCTTCCT TCCACTCCCC |
| 4201 | TCCCTGCCAG AGTCTGTCTT GGCCAGTGCC AGCCTCGATG CTTTGGTTTT GACCCCACCT |
| 4261 | GATCCTCCTT TCCTCATGCA GCACAAGTGC TCACCGGGGC CAGAGCCAGG GCATGGATAT |
| 4321 | GACAAGCAGG GCAGCCTGGA CACTGCCCTC ACAGGACAGC GCCAATAACA ATACAGTGTC |
| 4381 | TGAGTATCTC CAGGGGATGA TTTCTGGCTC TTTGTCTCCA ATCAGTCCCA CTCCCTCCTG |
| 4441 | AGGTCCCCAA GGGCAGTATT CAGAGAGGTT TCCTGCGTTT TATTTCTATT TGGTATACCC |
| 4501 | TCCACTGTTG TCCACTGCCC TGTGTGGCCT TCTGGTTGAC CTCTGCCCGA TCTTCTGTCT |
| 4561 | CTCTGAGGGA ATCAGAGTCC AGCATCCAGC CCCAGCTGGA ACAGCTGAAG TCACAAGCCT |
| 4621 | CCTCTAAGCC AAGGCCAGTG TGTTCAGAGG TGACTGCCAC CCATACTAGG ACAAACACAG |
| 4681 | CTCAGATCAC CAGGTCAAGC ACCTAGGCCT GGCTTCTCCT GAGACAGAGG ACTCAGAAGT |
| 4741 | GGCCTTTCCT CCAAAGCCTG CTCAGACACA GGTCTGTAGG GCCAGGGTGT TCTGCTTGGC |
| 4801 | TGGGCTGCAG CTGCTACCCC TCGGTTGGGG CTGAGTCAGC CAGATCCTCC CCCTACTTCT |
| 4861 | CCCCAAGGGC CAAGAACTGC TCAGGGACAT TAAAGGTCAA AAGTCCAGCC ACACTCATTC |
| 4921 | ATCCTTTCCC CAGGCCCATG AAGAGAGGCA TCTCATTGTA GAATGTATGA GGAAGTGGGA |
| 4981 | AGTATCTCAG AGAATCAGCT AAGTTTCCTA ACTTGTCCAT CCAAATGTGA TCACCACGAT |
| 5041 | TCAACAATTT GGGGCATTGC TGATCTAGCC GTTCCTAGTG GGGCTTGCTC AAGGTTGCAC |
| 5101 | AGCGAGTCAG TAGAAGCCCT GGCTGGCCCC ACTTGGTACC AATCCACCAG GCAGCTCAGG |
| 5161 | GCTCCTGCCC AGCCCAGCAG CTTCTGTTGT CTAACGTATG GCAGGCAGAC TGGGAGCAGG |
| 5221 | AAAACAGAGG GCCCCAAAGC CCAAGGCACC AGAAGGTTTG TTTCAGTTTG CTGAAGCTGA |
| 5281 | TTTGTAATGA TTGGCACTCT TCAGCCAGGG GAGTGGGTAG GCCATAGCCA AGGATCGATT |
| 5341 | CCCCAACCAC AGCAAAGGCA ACACTCTTCC TCCAGAGATC ACCAAGCCCC TCTTACCTCC |
| 5401 | CTCCCTCCTT CCCAAGGCTG GCACTAACCA GGTACCACAT TCATTGTTAA GGAATGGCTG |
| 5461 | ATGACTGCTA CACGTGTTGG GAACCTGGTT GGGGCTGTGC AGTTTGGGCT GGAAGGAGAG |
| 5521 | ATGCCAGCCC TCGTGCTGCC TCTGGTCCCT GAAGTGTCAC CTCTCTCAGG ACCTCTCCTC |
| 5581 | TGGCCTGTGG GGTTATAAGT GATGGATAGC AGAAAGGGAG AACTGACTCC TGTCCCAAAT |
| 5641 | AGCTCCTCTG CCACCTGTCC TGCAGTGGGC CTGTGTGGGT TATGATTCTA GATCCTAGAC |
| 5701 | AGAGGCTGGG TCAGCTGTGG ATGGGGTGGT GCCTTGGTCT CTCTTGACTA CCTCGTCCAA |
| 5761 | AGAGAGCACT GCCCTTAGAC AAGAGTTGCT TGTCCTGCTG TGGGCTGGGC TTCCAGCTGC |
| 5821 | AGACCTCCAG TTGCTTGGTG TTCACTTTGC TCCTCTTGCC CTCTGTCTTC TGGTCCAGGC |
| 5881 | AGATCAGGGG CTCTGGGGAA ACTGCTGGAA CTCGAGGTGA GGATCAGCCT TTTCCAGCAT |
| 5941 | CCTGTGAGAG ACCAGAGAGA GAGTTTGGAT TTCATGTGGG GAACCCTCAA GGCCTGTCTG |
| 6001 | GAGAAGTGAC ACAGGATTTA CTGGGGTGGG CTGGTCCAGG TAGCTCTCCT GAACCTCCTC |
| 6061 | CTTCCCCAAG CTGAGAAGCT GAGAGCTGGA GGACAATATC CAGGGACATG GCTCTGGAAA |
| 6121 | ATAACTTTTT TTTTTTTAAG AGACAGGGTC TTGCTCTGTT GTCCAGGCTG GAGGGCAGTG |
| 6181 | ACATAATCAT AGCTCACTGT ACCCTTGAAC TCCTGGGCTC AAGTGATCCT CCTGCCTCAG |
| 6241 | CCTCCTTAGT AGCTGGGACT ACCAGTGCAT ACCACCATGC CTGGGTGATT TTTTAAATTT |
| 6301 | TTTATACAGA CAAGGTCTTG CTATGTTGCC CAGGCTGATC TTGAATTCCC GGGCTCAAGT |
| 6361 | GGTCCTCCTG CCTCAGCCTC CCACAGGATC GGGATTACAG GCAAGAGCCT CCACGCCCGG |
| 6421 | CCATGAAATA TAATTCTTAA TATCATACAG GAAAAAGTCA GCGGGTCAAG CTAGCCTGTG |
| 6481 | GCCCAGCCAC AACTAGCTGA CAAAGCTTCC TGGCCTTCCC TTTAACACAG TTCTGCTGCC |
| 6541 | ATAGTTCCAT CTATAAAATG GGAATGGAGG GAAATAGGGG AACTGGGAGA GAGAACACAG |
| 6601 | CCTTGCCAAG CAGCAATGTT AGCCTGATCC TTCCTCCACC TAGCTCGCCA TCTCGCCCTT |
| 6661 | GGAAAATGGC TCCTGGAGGA TTAGGCAGCC ATCTGCAAGG AGAGGGGCAA CCTGGGACAA |
| 6721 | GACACCCAGA GGGTAAGGAT TCCAGGAATG AAGCTGCCAT TTCTGGTTGG GAGGAGAAGA |
| 6781 | GGAAACTTTT AAGAGAAAGG GCTCCATTAT GAGCATGGGT TCAGGGCCCT GCATTACCCA |
| 6841 | ATCAGAACAG CCGGGATGAG CAGGAGGCCA GCTCCCAGGA GGAAGGGGAA CCCCTTCATA |
| 6901 | AAGTTCAGAG TGGCTGGGTA GAGTGAGTTG AAGATGCCGG AGGCCGTCAG CATGGCCAGG |
| 6961 | CTATTCACAC AGGCCACAGC AGAAAAGAGA GCACCTGTGA AGAAATAAAT ACCATACTCT |
| 7021 | GGAGTCCGAA AGGGCCATAT TCCAACTCTG GCACCACCAC CTCACAGCTG TGTGACCGGG |
| 7081 | AGTAGTCACT TAACCTATGT CTCCCCTTCC TCACCAGTAA ATCCTGCTAC ATCATGTACT |
| 7141 | GTGACAAGGA TTCAGTAAGG TCATATGTGG ACAGTAGCTG GCACAGAGGG GCTACTAAAC |
| 7201 | AAATGGCTGC TATTAAATCC ACATTAAAAG TACATGTGAT CTGACAGAAC CCAGCACATA |
| 7261 | AAAGAAAAAA AAAGTACATG TGATATTGTC TGATGAAAGC TTGATGGAAA TGGCTTTTTT |
| 7321 | CTGGTTTATC CTCTTTGGAA TCATCTCCTG TTTGGGATTA ACTGCTGGTC TGATCAGTTC |
| 7381 | CAATATTCAT AGCGGTGTCA CCACTGAATA GCTTCTTATC CTTTGGGTTC CTGTTCCTCC |
| 7441 | TTCTGCTAAA TAAGGATAAT ACCTATTTCC TAGATTGTGA GCAACATTAA GTTCACATGG |
| 7501 | AAATCACCCA TCACTGGGCC TGGTCCCCTG GAAGTAGCTA GTTAGTAAGG GCTGTTCTTT |
| 7561 | TCTCCTGTTT CTCTTGACAT CTCTGGGCAC AGAGAAAGTG CTGGGAAAAA AAGTTTAGGT |
| 7621 | GAATGAATGA AGACACATGG ATTCTGGGGA CACCAGAACC CACAGTGGGC TCTGTATGGC |
| 7681 | ACCAGAGTCT CTGTCATCAT CAGATCCTCA TTCCAGGACA GATGGAAAAA GATGAATGTT |
| 7741 | TCCAGACTGG GGCATAAAGA CCCAGAGGCT GGAGAAGCTG TTCTTTATAG ATATACCAGG |
| 7801 | AGAACCCACA GTTTACAAAA TGTGCAACAA CCCAACAGAA GTTGAGATTA AATTCTGTCA |
| 7861 | CATCTAGAGG GGTCTGTGAT GTCATCAAAA GCAAACCACC CACATCACAG ATGAAGAAAC |
| 7921 | AGGCCTGTGG CAGGGCTCGG ACTAAAACCC AGATCCTGAG ACCAGCTGCT TTTAAACACA |
| 7981 | GACGTAGGTT TGCATCCTAG CTCCACCATT TACTGAGTAA CCTTGGGTGA GCCAATGTAA |
| 8041 | CCCCCTGGGT CTCTGTTTCT TTATCTGTCA ACTGTGGAAA ATGAAACCCA TGTCACAAGG |
| 8101 | TTGTTCACTT CTGGGCTTGT ACACGCTGAC CCCAGAGAAA CAGGGAACTC TGGCATCACC |
| 8161 | ACACCCATCT TACAGACGGA AAAGCTGAGG TCTGCAGAGA GTAAATCCTC TGCTCTGGTT |
| 8221 | ATCTAGAAAG AACATAATTG TGCTCTGCTG ACTGCAAATC CCAACTCTGC GGTTTGAAAA |
| 8281 | TCCAAGGTGG CATGATCCTC TGCCCATTGT GGGCAATTTC ACAGAAATGT GTTTGTTTTG |
| 8341 | GCCACTTACT TCTCCAGGGT GAGAGGGGGG AAGGCAAGCT GTTCCCCCAG CCATGGCTGC |
| 8401 | CCATCAGCCC GTTTCGGGCA GCACTGGACA TGAGGAACCA GACACAGGTG GGTTCTGACA |
| 8461 | CTCACCCTGC TCTGTCTCTC TCACCAGCTT GGAGAGTTTA GCCCGGATGA CAGGTGTGAT |
| 8521 | GACTAATGAC AGGAAAAGCA ACCCATATCC TGTGGAGAAA CAAACACTCA TCAGGAAAAT |
| 8581 | GGGGCTGGGG AGAGGGGCGT CCAAGGGAAA GGCAGCAGAG CTCCTATCCA TACCCCACGT |
| 8641 | GGGGCTTAGG TTAGACCCAG GAAGAACTTC CTTGATGGTG AGGGTGGGAA GACAGTAGTC |
| 8701 | AAGGAGGAAT GGAGACTGCC CTTGTCTGGG CTTGGCCACC TGCTAGCTCT CATGAATGAA |
| 8761 | TGCTAATTCC CATTGATTGC TTTCTTGTCT GAACCTCTTG TGGTCACAGC AGGCATCACC |
| 8821 | CACCCACTTG GCACTTAGTA GGGATATGGC AGGGCACAGA AAACAAGCAT GGGCTTTGGA |
| 8881 | GTCAGCCCTG AGTTCAAAAC CTGATGCCAT TACATATTAT CTGTGTGGCC TGGGGTACTT |
| 8941 | ACCCTCTCTG ATCCTGACTC CCTGTATGAG GAAGATAATA AGGCCTTCAT CACAGGATGG |
| 9001 | TTCTGAGGCA TAGGAGGCTG AATAATGGTG CCCAATGGCA TCAGATTCAT AGCCCTGGAA |
| 9061 | CCTGTAAATA CTACCTTATT TGGAAAATGA GTCTATGCAG GTGTGCAGTT AAGCCTCCTG |
| 9121 | AGAGAGCAGA GTTATCCTGG ATTAGGTTGG GCCCTAAATG CCGTCACACA TATCTTTATA |
| 9181 | AGAGGAAAGC AGACGGAGAT TTGGCACCGA CAGAATTGAG AAGGCACAAA GAGGAGGAGA |
| 9241 | GTCAATGTGA GCACAGAGGC AGAGACTGGT GATGGCCGCC CCAAGCCAAG GAATGCCAGC |
| 9301 | AGCCCCAGAA GCTGGAAGAA ATGAGAAACA CGTTCTCTCC TGGAGGCTTG CAAGGGAGCA |
| 9361 | CTGCCTGCTG ACTGCTTCCA TTCAGCCCGG TGGTACTGAC TTTGGACTTC TGGCCTCCAG |
| 9421 | AACTGTGAGA GAATATGTTT CTGTTGTGTT AAGCCCCCAA GTTTGTGGTA TGTCATTACA |
| 9481 | GCAATCTCAG GGAACCAATA CATGAGGTAA AAAGGTAACA TCTATGAAGA GCATGGCATA |
| 9541 | GGGACACAGC AAATGGGAGT TCCTTTTCCC TTTGCATTCA GTTACTTACA GGCTTCCTGT |
| 9601 | TTTCTTCATA ACCATTICTC TCCCTGTGCG ACTGCTGACT CCTCAGCAAA ACTGCAAACT |
| 9661 | CCTACAGGAC AGTGGATCCT CCAAAGAAGG TATACGATGA GGCATCCAGG GACCCTAGCA |
| 9721 | GTGTCAGGCC CCTCAAATCC CACTCTGTTG AGACCTCCCC CCGACCCAGA GCAATGACAG |
| 9781 | CATCTTTATC ATCTCTGCAT CCCCCAGGGC CATCAGCAGG AGGGAAAGGT TCCCTTCTGC |
| 9841 | TTAATTGTCA GACAAGCAGT TGAGTTAAGA AATCTGTGAT TATTGTATTG TTGACTATAC |
| 9901 | ACAGCACATT TTAGGGCTCT ATCAAAATAA ATCTGTCCCT TTAAAAAAAG TTAACTAAAG |
| 9961 | CCGGGCACGG TGGCTCATGC CTGTAATCCC AACACTTTGG GAGGCTGAGG CAGGCGGATC |
| 10021 | CTTGAGCTCA GGAGTTAGAG ACCTGGACTG GGCAAAATGG TGAGGACCCC ATCTCTATAA |
| 10081 | AAAATACAAA AATTAGCAAG GTGTGGTAAT GTGCACCAGT GGTCCCAGCT ACTAGAGAGG |
| 10141 | CCAAGGTGGG AGGATCATCT GGGCCCGGGG GATGAGGCTG CAGTGAGCCA TGATCGTGCC |
| 10201 | ACTGCACTCT AGCCTGGGTA ACAAAGCGAG ACCCTGTCTC TAAATACATC AATCAAATAA |
| 10261 | AAATTTTAAA AAGTTAA. |

The corresponding amino acid sequence of human SARM1 protein can be found at NP_055892.2:

| (SEQ ID NO: 65) | |
|---|---|
| 1 | MVLTLLLSAY KLCRFFAMSG PRPGAERLAV PGPDGGGGTG PWWAAGGRGP REVSPGAGTE |
| 61 | VQDALERALP ELQQALSALK QAGGARAVGA GLAEVFQLVE EAWLLPAVGR EVAQGLCDAI |
| 121 | RLDGGLDLLL RLLQAPELET RVQAARLLEQ ILVAENRDRV ARIGLGVILN LAKEREPVEL |
| 181 | ARSVAGILEH MFKHSEETCQ RLVAAGGLDA VLYWCRRTDP ALLRHCALAL GNCALHGGQA |
| 241 | VQRRMVEKRA AEWLFPLAFS KEDELLRLHA CLAVAVLAIN KEVEREVERS GTLALVEPLV |
| 301 | ASLDPGRFAR CLVDASDTSQ GRGPDDLQRL VPLLDSNRLE AQCIGAFYLC AEAAIKSLQG |
| 361 | KTKVFSDIGA IQSLKRLVSY STNGTKSALA KRALRLLGEE VPRPILPSVP SWKEAEVQTW |
| 421 | LQQIGFSKYC ESFREQQVDG DLLLRLTEEE LQTDLGMKSG ITRKRFFREL TELKTFANYS |
| 481 | TCDRSNLADW LGSLDPRFRQ YTYGLVSCGL DRSLLHRVSE QQLLEDCGIH LGVHRARILT |
| 541 | AAREMLHSPL PCTGGKPSGD TPDVFISYRR NSGSQLASLL KVHLQLHGFS VFIDVEKLEA |
| 601 | GKFEDKLIQS VMGARNFVLV LSPGALDKCM QDHDCKDWVH KEIVTALSCG KNIVPIIDGF |
| 661 | EWPEPQVLPE DMQAVLTFNG IKWSHEYQEA TIEKIIRFLQ GRSSRDSSAG SDTSLEGAAP |
| 721 | MGPT. |

As used herein, the term “SARM1-mediated neurological disease” refers to a neurological disease, disorder, or injury mediated by SARM1 and/or by axonal degeneration.

The term “% sequence identity” or “percentage sequence identity” with respect to a reference nucleic acid sequence is defined as the percentage of nucleotides, nucleosides, or nucleobases in a candidate sequence that are identical with the nucleotides, nucleosides, or nucleobases in the reference nucleic acid sequence, after optimally aligning the sequences and introducing gaps or overhangs, if necessary, to achieve the maximum percent sequence identity. Alignment for purposes of determining percent nucleic acid sequence identity can be achieved in various ways that are within the skill in the art, for instance, using publicly available computer software programs, for example, those described in Current Protocols in Molecular Biology (Ausubel et al., eds., 1987, Supp. 30, section 7.7.18, Table 7.7.1), and including BLAST, BLAST-2, ALIGN, Megalign (DNASTAR), Clustal W2.0 or Clustal X2.0 software. Those skilled in the art can determine appropriate parameters for measuring alignment, including any algorithms needed to achieve maximal alignment over the full length of the sequences being compared. Percentage of “sequence identity” can be determined by comparing two optimally aligned sequences over a comparison window, where the fragment of the nucleic acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical nucleotide, nucleoside, or nucleobase occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence.

The term “polypeptide” or “protein”, as used herein, refers to a polymer of amino acid residues. The term applies to polymers comprising naturally occurring amino acids and polymers comprising one or more non-naturally occurring amino acids.

As used herein, “RNAi,” “RNAi agent,” “iRNA,” “iRNA agent,” or “RNA interference agent” means an agent that mediates sequence-specific degradation of a target mRNA by RNA interference, e.g., via RNA-induced silencing complex (RISC) pathway. In some embodiments, the RNAi agent has a sense strand and an antisense strand, and the sense strand and the antisense strand form a duplex (e.g., a double stranded RNA).

As used herein, “strand” refers to a single, contiguous sequence of nucleotides linked together through internucleotide linkages (e.g., phosphodiester linkages or phosphorothioate linkages). A strand can have two free ends (e.g., a 5′ end and a 3′ end).

As used herein, “treatment” or “treating” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

The following examples are offered to illustrate, but not to limit, the claimed inventions.

### EXAMPLES

### Example 1: Generation and Characterization of TfR Binding Proteins

### Generation of Human TfR Binding Proteins

Antibody against human TfR was generated by immunizing AlivaMab® transgenic mice with the extracellular domains of human Transferrin Receptor 1 protein with a His tag (hTfR-ECD-6His, SEQ ID NO: 67, see Table 8) and mouse Transferrin Receptor protein (mTfR, SEQ ID NO:66). Antigen positive B-cells were sorted from pooled spleens. Binding of individual antibodies cloned from those B-cells to his-tagged hTfR-ECD was verified.

Additional antibody against human TfR was generated by immunizing AlivaMab® transgenic mice with the apical domain of human Transferrin Receptor 1 protein with a His tag (hTfR-ApD-6His, SEQ ID NO: 68, see Table 8). Antigen positive B-cells were sorted from pooled spleens. Binding of individual antibodies cloned from those B-cells to his-tagged hTfR-ECD was verified.

TABLE 8
Sequences of the immunogens used to generate human or mouse TfR
antibodies.

| Immunogen | Sequence | SEQ ID NO |
|---|---|---|
| mTfR-ECD-6His | HHHHHHCKRVEQKEECVKLAETEETDKSETMETEDV | 66 |
| | PTSSRLYWADLKTLLSEKLNSIEFADTIKQLSQNTYTP | |
| | REAGSQKDESLAYYIENQFHEFKFSKVWRDEHYVKI | |
| | QVKSSIGQNMVTIVQSNGNLDPVESPEGYVAFSKPTE | |
| | VSGKLVHANFGTKKDFEELSYSVNGSLVIVRAGEITF | |
| | AEKVANAQSFNAIGVLIYMDKNKFPVVEADLALFGH | |
| | AHLGTGDPYTPGFPSFNHTQFPPSQSSGLPNIPVQTISR | |
| | AAAEKLFGKMEGSCPARWNIDSSCKLELSQNQNVKL | |
| | IVKNVLKERRILNIFGVIKGYEEPDRYVVVGAQRDAL | |
| | GAGVAAKSSVGTGLLLKLAQVFSDMISKDGFRPSRSII | |
| | FASWTAGDFGAVGATEWLEGYLSSLHLKAFTYINLD | |
| | KVVLGTSNFKVSASPLLYTLMGKIMQDVKHPVDGKS | |
| | LYRDSNWISKVEKLSFDNAAYPFLAYSGIPAVSFCFCE | |
| | DADYPYLGTRLDTYEALTQKVPQLNQMVRTAAEVA | |
| | GQLIIKLTHDVELNLDYEMYNSKLLSFMKDLNQFKT | |
| | DIRDMGLSLQWLYSARGDYFRATSRLTTDFHNAEKT | |
| | NRFVMREINDRIMKVEYHFLSPYVSPRESPFRHIFWGS | |
| | GSHTLSALVENLKLRQKNITAFNETLFRNQLALATWT | |
| | IQGVANALSGDIWNIDNEF | |
| hTfR-ECD-6His | HHHHHHCKGVEPKTECERLAGTESPVREEPGEDFPA | 67 |
| | ARRLYWDDLKRKLSEKLDSTDFTGTIKLLNENSYVPR | |
| | EAGSQKDENLALYVENQFREFKLSKVWRDQHFVKIQ | |
| | VKDSAQNSVIIVDKNGRLVYLVENPGGYVAYSKAAT | |
| | VTGKLVHANFGTKKDFEDLYTPVNGSIVIVRAGKITF | |
| | AEKVANAESLNAIGVLIYMDQTKFPIVNAELSFFGHA | |
| | HLGTGDPYTPGFPSFNHTQFPPSRSSGLPNIPVQTISRA | |
| | AAEKLFGNMEGDCPSDWKTDSTCRMVTSESKNVKL | |
| | TVSNVLKEIKILNIFGVIKGFVEPDHYVVVGAQRDAW | |
| | GPGAAKSGVGTALLLKLAQMFSDMVLKDGFQPSRSII | |
| | FASWSAGDFGSVGATEWLEGYLSSLHLKAFTYINLD | |
| | KAVLGTSNFKVSASPLLYTLIEKTMQNVKHPVTGQFL | |
| | YQDSNWASKVEKLTLDNAAFPFLAYSGIPAVSFCFCE | |
| | DTDYPYLGTTMDTYKELIERIPELNKVARAAAEVAG | |
| | QFVIKLTHDVELNLDYERYNSQLLSFVRDLNQYRADI | |
| | KEMGLSLQWLYSARGDFFRATSRLTTDFGNAEKTDR | |
| | FVMKKLNDRVMRVEYHFLSPYVSPKESPFRHVFWGS | |
| | GSHTLPALLENLKLRKQNNGAFNETLFRNQLALATW | |
| | TIQGAANALSGDVWDIDNEF | |
| hTfR-ApD-6His | HHHHHHHHGKPIPNPLLGLDSTGGGGSDSAQNSVIIV | 68 |
| | DKNGRLVYLVENPGGYVAYSKAATVTGKLVHANFG | |
| | TKKDFEDLYTPVNGSIVIVRAGKITFAEKVANAESLN | |
| | AIGVLIYMDQTKFPIVNAELSFFGHAHLGGGGGGLPN | |
| | IPVQTISRAAAEKLFGNMEGDCPSDWKTDSTCRMVTS | |
| | ESKNVKLTVS | |

Affinity variants of the generated human TfR antibodies were made by systematically introducing mutations into individual CDR of each antibody and the resulting variants were subjected to multiple rounds of selection with decreasing concentrations of antigen and/or increasing periods of dissociation to isolate clones with improved affinities. The sequences of individual variants were used to construct a combinatorial library which was subjected to an additional round of selection with increased stringency to identify additive or synergistic mutational pairings between the individual CDR regions. Individual combinatorial clones are sequenced. The heavy chain and light chain CDRs and VH/VL sequences of the human TfR binding domains and proteins are provided in Table 1a.

Human TfR binding proteins were generated by recombinant DNA technology. Such human TfR binding proteins can be expressed in a mammalian cell line such as HEK293 or CHO, either transiently or stably transfected with an expression system using an optimal predetermined HC: LC vector ratio or a single vector system encoding both HC and LC. Clarified media, into which the protein has been secreted, can be purified using the commonly used techniques.

### Binding Affinity

Binding affinity and binding stoichiometry of the exemplified human TfR binding proteins to human and cynomolgus TfR was characterized using a surface plasmon resonance assay on a Biacore 8K instrument primed with HBS-EP+ (10 mM Hepes pH7.4+150 mM NaCl+3 mM EDTA+0.05% (w/v) surfactant P20) running buffer and analysis temperature set at 37° C. Target human and cynomologus TfR ECD's were immobilized on a CM4 chip (Cytiva P/N 29104989) using standard NHS-EDC amine coupling. The TfR binding proteins were prepared at a final concentration of 0.3, 0.1, 0.033, 0.01, 0.0033, 0.001, 0.00033, 0.0001 μM respectively by dilution of stock solution into running buffer.

Binding analysis was performed in a multi-cycle kinetics manner. Each analysis cycle consists of (1) injection of the lowest to highest concentration proteins over all Fc at 50 μL/min for 140 seconds followed by return to buffer flow for 400 seconds to monitor dissociation phase; (2) regeneration of chip surfaces with injection of 3M magnesium chloride, for 30 seconds at 100 μL/min over all cells; and (3) equilibration of chip surfaces with a 50 μL (30-sec) injection of HBS-EP+. Data were processed using standard double-referencing and fit to a 2-state binding model using Biacore 8K Evaluation software, to determine the association rate (kₒₙ, M⁻¹ s⁻¹ units), dissociation rate (k_{off}, s⁻¹ units), and Rₘₐₓ (RU units). The equilibrium dissociation constant (K_{D}) is calculated from the relationship K_{D}=k_{off}/kₒₙ, and is in molar units. Results are provided in Table 9.

TABLE 9
Binding Affinity of Exemplified human TfR binding
proteins to human or cynomolgus TfR at 37° C.

| | | Standard error | | Standard error of |
|---|---|---|---|---|
| Human TfR | | of the mean, | | the mean, Cyno |
| binding | Human TfR K_{D} | Human TfR K_{D} | Cyno TfR K_{D} | TfR K_{D} |
| proteins | (Biacore, nM) | (Biacore, nM) | (Biacore, nM) | (Biacore, nM) |
| (TBP) | at 37° C. | n = 3 | at 37° C. | n = 3 |
| TBP3 | 32.087 | 11.795 | 66.565 | 11.695 |
| TBP4 | 153.642 | 7.949 | 300.180 | 2.565 |
| TBP5 | 0.522 | 0.284 | 502.210 | 8.129 |

### Example 2: Synthesis and Characterization of dsRNAs Targeting SARM1

Single strands (sense and antisense) of the dsRNA duplexes were typically synthesized on solid support via a MerMade™ 12 (LGC Biosearch Technologies) or a similar automated oligonucleotide synthesizer. In some cases the sense strands were synthesized using an appropriate CPG such as 3′-Cholesterol-TEG CNA CPG 500 (LGC Biosearch Technologies) The sequences of the sense and antisense strands were shown in Table 5 or 7.

Standard reagents were used in the oligo synthesis (Table 10), where 0.1M xanthane hydride in pyridine was used as the sulfurization reagent and 20% DEA in ACN was used as an auxiliary wash post synthesis. All monomers (Table 11a) were made at 0.1M in ACN and contained a molecular sieves trap bag.

The oligonucleotides were cleaved and deprotected (C/D) at 45° C. for 20 hours. The sense strands were C/D from the CPG using ammonia hydroxide (28-30%, cold), whereas 3% DEA in ammonia hydroxide (28-30%, cold) was used for the antisense strands. C/D was determined complete by IP-RP LCMS when the resulting mass data confirmed the identity of sequence. Dependent on scale, the CPG was filtered via 0.45 um PVDF syringeless filter, 0.22 um PVDF Steriflip® vacuum filtration or 0.22 um PVDF Stericup® Quick release. The CPG was back washed/rinsed with either 30% ACN/RNAse free water or 30% EtOH/RNAse free water then filtered through the same filtering device and combined with the first filtrate. This was repeated twice. The material was then divided evenly into 50 mL falcon tubes to remove organics via Genevac™. After concentration, the crude oligonucleotides were diluted back to synthesized scale with RNAse free water and filtered either by 0.45 um PVDF syringeless filter, 0.22 um PVDF Steriflip® vacuum filtration or 0.22 μm PVDF Stericup® Quick release.

The crude oligonucleotides were purified via AKTA™ Pure purification system using either anion-exchange (AEX) or reverse phase (RP) a source 15Q-RP column. For AEX, an ES Industry Source™ 15Q column maintaining column temperature at 65° C. with MPA: 20 mM NaH₂PO₄, 15% ACN, pH 7.4 and MPB: 20 mM NaH₂PO₄, 1M NaBr, 15% ACN, pH 7.4. For RP, a Source™ 15Q-RP column with MPA: 50 mM NaOAc with 10% ACN and MPB: 50 mM NaOAc with 80% ACN. In all cases, fractions which contained a mass purity greater than 85% without impurities >5% where combined.

The purified oligonucleotides were desalted using 15 mL 3K MWCO centrifugal spin tubes at 3500×g for ˜30 min. The oligonucleotides were rinsed with RNAse free water until the eluent conductivity reached <100 usemi/cm. After desalting was complete, 2-3 mL of RNAse free water was added then aspirated 10×, the retainment was transferred to a 50 mL falcon tube, this was repeated until complete transfer of oligo by measuring concentration of compound on filter via nanodrop. The final oligonucleotide was then nano filtered 2× via 15 mL 100K MWCO centrifugal spin tubes at 3500×g for 2 min. The final desalted oligonucleotides were analyzed for concentration (nano drop at A260), characterized by IP-RP LCMS for mass purity and UPLC for UV-purity.

For the preparation of duplexes, equimolar amounts of sense and antisense strand were combined and heated at 65° C. for 10 minutes then slowly cooled to ambient temperature over 40 minutes. Integrity of the duplex was confirmed by UPLC analysis and characterized by LCMS using IP-RP. All duplexes were nano filtered then endotoxin levels measured via Charles River Endosafe® Cartridge Device to give the final compounds of RNAi agent. For in vivo analysis, the appropriate amount of duplex was lyophilized then reconstituted in 1×PBS for rodent studies and a CSF for non-human primate studies.

TABLE 10
Oligonucleotide Synthesis Reagents
Reagents

Activator Solution (0.5M ETT in ACN)
Cap A (Acetic Anhydride, Pyridine in THF, 1:1:8)
Cap B (1-Methylimidazole in THF, 16:84)
Oxidation Solution (0.02M Iodine in THF/Pyridine/Water,
70:20:10)
Deblock Solution, 3% TCA in DCM (w/v)
Acetonitrile (Anhydrosolv, Water max. 10 ppm)
Xanthane Hydride (0.1M in Pyridine)
Diethylamine (20% in Acetonitrile)

TABLE 11a
Phosphoramidites

| Phosphoramidite | Abbreviation | Supplier | Catalog # | CAS |
|---|---|---|---|---|
| DMT-2′-F-A(Bz)- | fA | Hongene | PD1-001 | 136834-22-5 |
| CE | | | | |
| Phosphoamidite | | | | |
| DMT-2′-F-C(Ac)- | fC | Hongene | PD3-001 | 159414-99-0 |
| CE | | | | |
| Phosphoamidite | | | | |
| DMT-2′-F- | fG | Hongene | PD2-002 | 144089-97-4 |
| G(iBu)-CE | | | | |
| Phosphoamidite | | | | |
| DMT-2′-F-U-CE | fU | Hongene | PD5-001 | 146954-75-8 |
| Phosphoamidite | | | | |
| DMT-2′-O-Me- | mA | Hongene | PR1-001 | 110782-31-5 |
| A(Bz)-CE | | | | |
| Phosphoamidite | | | | |
| DMT-2′-O-Me- | mC | Hongene | PR3-001 | 199593-09-4 |
| C(Ac)-CE | | | | |
| Phosphoamidite | | | | |
| DMT-2′-O-Me- | mG | Hongene | PR2-002 | 150780-67-9 |
| G(iBu)-CE | | | | |
| Phosphoamidite | | | | |
| DMT-2′-O-Me- | mU | Hongene | PR5-001 | 110764-79-9 |
| U-CE | | | | |
| Phosphoamidite | | | | |
| 5′bis(POM) vinyl | POM-VPmU | Hongene | PR5-032 | BVPMUP23B2A1 |
| phosphate-2′- | | | | |
| Ome-U3′CE | | | | |
| phosphoroamidite | | | | |

TABLE 11b
Cholesterol Conjugate Structure

| | Structure |
|---|---|
| 1 (Cholesterol conjugate) | 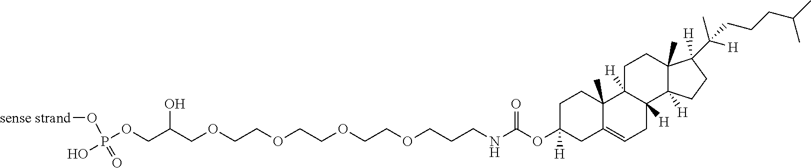 |

### Example 3: Generation of SARM1 RNAi Agents

Certain abbreviations are defined as follows: “ACN” refers to acetonitrile; “aAEX” refers to analytical anion exchange; “AS” refers to antisense strand; “DAR” refers to drug/siRNA to antibody/protein ratio; “DCM” refers to dichloromethane; “DHAA” refers to dehydroascorbic acid; “dsRNA” refers to double stranded ribonucleic acid; “DTT” refers to dithiothreitol; “h” refers to hours; “HPLC” refers to high-performance liquid chromatography; “LC/MS” refers to liquid chromatography mass spectrometry; “LTQ/MS” refers to linear ion trap mass spectrometer; “min” refers to minutes; “MW” refers to molecular weight; “MWCO” refers to molecular weight cut-off; “NHS” refers to N-hydroxysuccinimide; “OD” refers to optical density; “PBS” phosphate-buffered saline; “PEG” refers to polyethylene glycol; “RNAi” refers to RNA interference; “rpm” refers to revolutions per minute; “SEC” refers to size exclusion chromatography; “siRNA” refers to small interfering RNA; “SMCC” refers to succinimidyl-4-(N-maleimidomethyl)cyclohexane-1-carboxylate; “SS” refers to sense strand; “TCO” refers to trans-cyclo-octene; “TfR” refers to transferrin receptor; “THF” refers to tetrahydrofuran; “TRIS” refers to tris(hydroxymethyl)aminomethane; “UPLC” refers to ultra performance liquid chromatography; and “UV” refers to ultraviolet.

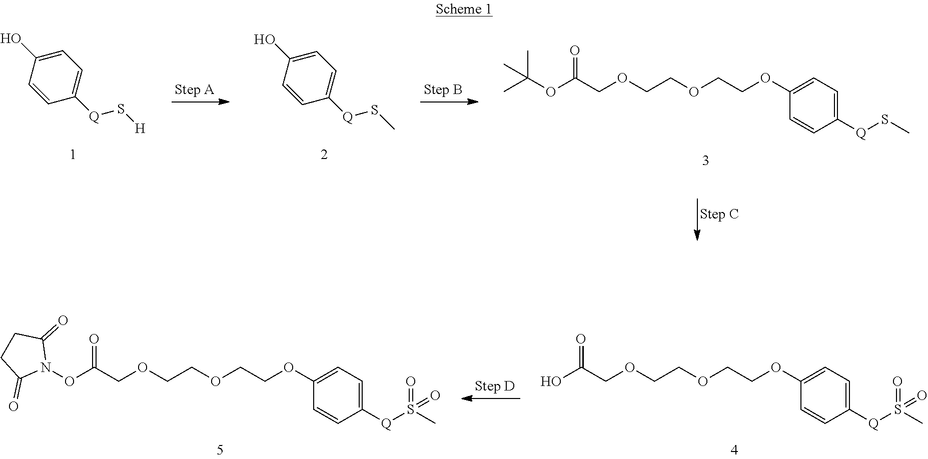

Q is 1,3,4-oxadiazole or 1H-tetrazole.

Scheme 1 depicts the synthetic route to the intermediates used to form the final MSPT and OD linkers shown in Table 4.

Scheme 1, step A depicts the methylation of the thiol on compound (1) using iodomethane and a suitable base such as DIEA in a solvent such as THF to give compound (2). Step B shows an alkylation of compound (2) with tert-butyl 2-(2-(2-bromoethoxy)ethoxy)acetate using a base such as potassium carbonate in a solvent such as acetone to give compound (3). Step C shows the oxidation of compound (3) with hydrogen peroxide and ammonium molybdate (VI) tetrahydrate in a solvent such as EtOH followed by an acidic deprotection using an acid such as TFA in a solvent such as DCM to give compound (4). Note that in the case of the 1H-tetrazole, the deprotection took place during the oxidation step. Step D depicts a coupling of compound (4) and 1-hydroxypyrrolidine-2,5-dione using EDCI in a solvent system such as DCM and THE to give compound (5).

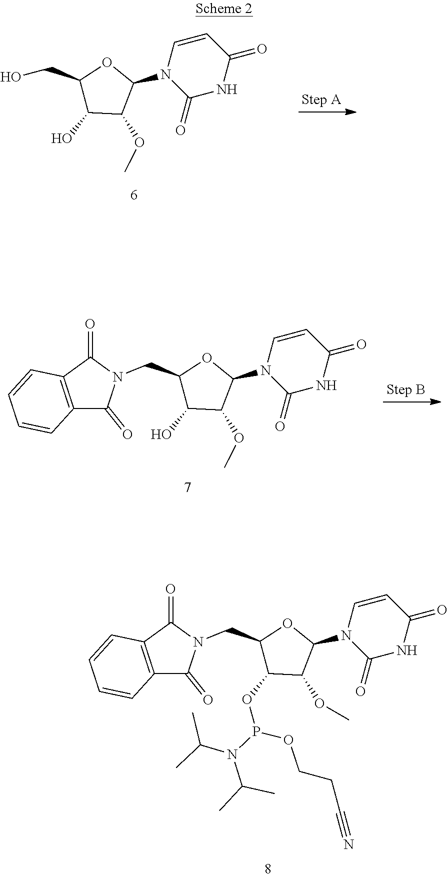 [SMILES: CO[C@@H]1[C@H](O)[C@@H](CO)O[C@H]1n1ccc(=O)[nH]c1=O ; CO[C@@H]1[C@H](O)[C@@H](CN2C(=O)c3ccccc3C2=O)O[C@H]1n1ccc(=O)[nH]c1=O ; CO[C@@H]1[C@H](OP(OCCC#N)N(C(C)C)C(C)C)[C@@H](CN2C(=O)c3ccccc3C2=O)O[C@H]1n1ccc(=O)[nH]c1=O]

Scheme 2, step A shows the coupling of compound (6) and isoindoline-1,3-dione using DIAD and tributyl phosphine in a solvent such as THE to give compound (7). Step B depicts the phosphorylation of compound (7) with 2-cyanoethyl-N,N-diisopropylchlorophosphoramidite using a base such as DIEA in a solvent such as DCM to give compound (8).

### Preparation 1

### 4-(5-(Methylthio)-1H-tetrazol-1-yl)phenol

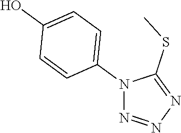

A solution of 4-(5-mercapto-1H-tetrazol-1-yl)phenol (4.00 g, 20.6 mmol) in THF (50 mL) was cooled to 0° C. DIEA (4.31 g, 33.3 mmol) was added then stirred for 10 minutes before adding iodomethane (1.54 mL, 24.7 mmol) dropwise over a period of 1 minute. The mixture was stirred at 0° C. for 20 minutes, and then stirred at ambient temperature for 12 hours. After this time, the mixture was diluted with EtOAc (100 mL) and washed with saturated aqueous NH₄Cl (2×50 mL). The organic layer was separated, dried over sodium sulfate, and concentrated in vacuo to give the title compound (4.2 g, 93%). ES/MS m/z: 209 (M+H).

The compound in Table 12 was prepared in a manner essentially analogous to that found in Preparation 1.

TABLE 12

| Prep | Name | Structure | ES/MS m/z |
|---|---|---|---|
| 2 | 4-(5-(Methylthio)- 1,3,4-oxadiazol-2- yl)phenol | 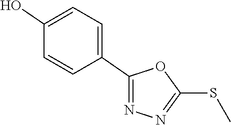 | 209 (M + H) |

### Preparation 3

### tert-Butyl 2-(2-(2-(4-(5-(methylthio)-1H-tetrazol-1-yl)phenoxy)ethoxy)ethoxy)acetate

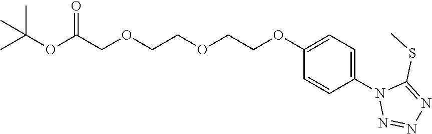 [SMILES: CSc1nnnn1-c1ccc(OCCOCCOCC(=O)OC(C)(C)C)cc1]

In a pressure vessel, potassium carbonate (3.15 g, 22.8 mmol) was added to tert-butyl 2-(2-(2-bromoethoxy)ethoxy)acetate (4.33 g, 14.8 mmol) and 4-(5-(methylthio)-1H-tetrazol-1-yl)phenol (2.5 g, 11.4 mmol) in acetone (60 mL). The pressure vessel was sealed and heated at 80° C. for 8 hours with vigorous stirring. After this time, the mixture was cooled to ambient temperature then filtered while washing through with acetone/EtOAc/DCM (30 mL each). The filtrate was concentrated in vacuo and purified via silica gel column chromatography eluting with 0-100% EtOAc/DCM to give the title compound as a white solid (3.98 g, 85%). ES/MS m/z: 411 (M+H).

The compound in Table 13 was prepared in a manner essentially analogous to that found in Preparation 3.

TABLE 13

| | | | ES/MS |
|---|---|---|---|
| Prep | Name | Structure | m/z |
| 4 | tert-Butyl 2-(2-(2-(4-(5- (methylthio)-1,3,4- oxadiazol-2- yl)phenoxy)ethoxy) ethoxy)acetate | 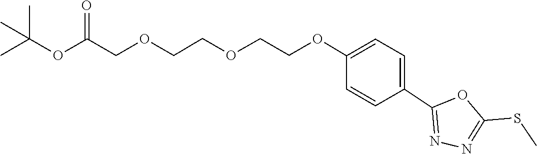 [SMILES: CSc1nnc(-c2ccc(OCCOCCOCC(=O)OC(C)(C)C)cc2)o1] | 411 (M + H) |

### Preparation 5

### 2-(2-(2-(4-(5-(Methylsulfonyl)-1H-tetrazol-1-yl)phenoxy)ethoxy)ethoxy)acetic acid

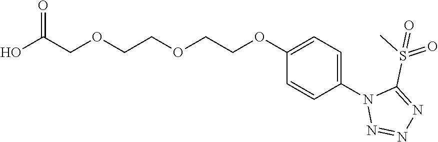 [SMILES: CS(=O)(=O)c1nnnn1-c1ccc(OCCOCCOCC(=O)O)cc1]

tert-Butyl 2-(2-(2-(4-(5-(methylthio)-1H-tetrazol-1-yl)phenoxy)ethoxy)ethoxy)acetate (3.98 g, 9.21 mmol) was dissolved in EtOH (100 mL) and cooled to 5-10° C. Then, 30% hydrogen peroxide (19 mL, 184 mmol) was added, followed by ammonium molybdate (VI) tetrahydrate (1.14 g, 0.921 mmol). The mixture was allowed to warm to ambient temperature and then stirred for 4 hours, after which it was diluted with DCM (150 mL) and washed with saturated aqueous sodium chloride solution. The organic phase was separated, dried over sodium sulfate, and concentrated in vacuo. The resulting residue was purified via silica gel column chromatography eluting with 0-100% EtOAc/DCM to give the title compound as a white solid (3.00 g, 80%). ES/MS m/z: 385 (M−H).

The compound in Table 14 was prepared in a manner essentially analogous to that found in Preparation 5.

TABLE 14

| | | | ES/MS |
|---|---|---|---|
| Prep | Name | Structure | m/z |
| 6 | tert-Butyl 2-(2-(2-(4-(5- (methylsulfonyl)-1,3,4- oxadiazol-2-yl)phenoxy) ethoxy)ethoxy)acetate | 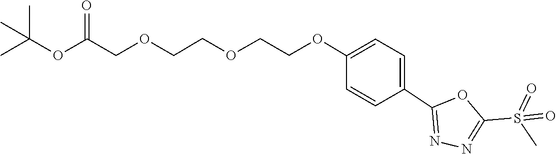 [SMILES: CC(C)(C)OC(=O)COCCOCCOc1ccc(-c2nnc(S(C)(=O)=O)o2)cc1] | 387 (M + H − tBu) |

Note
that the conditions were analogous, but the t-butyl group was not removed in the process.

### Preparation 7

### 2-(2-(2-(4-(5-(Methylsulfonyl)-1,3,4-oxadiazol-2-yl)phenoxy)ethoxy)ethoxy)acetic acid

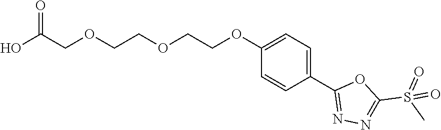 [SMILES: CS(=O)(=O)c1nnc(-c2ccc(OCCOCCOCC(=O)O)cc2)o1]

TFA (20 mL, 12.0 mmol) was added to a solution of tert-butyl 2-(2-(2-(4-(5-(methylsulfonyl)-1,3,4-oxadiazol-2-yl)phenoxy)ethoxy)ethoxy)acetate (5.60 g, 12.0 mmol) in DCM (60 mL). The mixture was stirred at ambient temperature for 2 hours, concentrated in vacuo, and purified via silica gel column chromatography eluting with 0-100% EtOAc/DCM to give the title compound (4.12 g, 82%). ES/MS m/z: 387 (M+H).

### Preparation 8

### 2,5-Dioxopyrrolidin-1-yl 2-(2-(2-(4-(5-(methylsulfonyl)-1H-tetrazol-1-yl)phenoxy)ethoxy)ethoxy)acetate

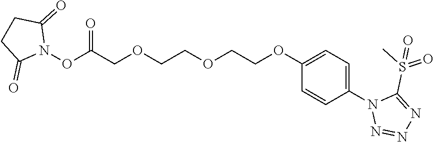 [SMILES: CS(=O)(=O)c1nnnn1-c1ccc(OCCOCCOCC(=O)ON2C(=O)CCC2=O)cc1]

EDCI (1.60 g, 10.3 mmol) was added to a solution of 2-(2-(2-(4-(5-(methylsulfonyl)-1H-tetrazol-1-yl)phenoxy)ethoxy)ethoxy)acetic acid (2.80 g, 7.25 mmol) and 1-hydroxypyrrolidine-2,5-dione (1.33 g, 11.6 mmol) in DCM (50 mL) and THF (70 mL). Another 20 mL of DCM was added to bring the mixture into a solution followed by stirring at ambient temperature for 12 hours. After this time, concentrated in vacuo and purified via silica gel column chromatography eluting with 0-100% EtOAc/DCM to give the title compound (2.61 g, 65%). ES/MS m/z: 484 (M+H).

The compound in Table 15 was prepared in a manner essentially analogous to that found in Preparation 8.

TABLE 15

| | | | ES/MS |
|---|---|---|---|
| Prep | Name | Structure | m/z |
| 9 | 2,5-Dioxopyrrolidin- 1-yl-2-(2-(2-(4-(5- (methylsulfonyl)-1,3,4- oxadiazol-2-yl)phenoxy) ethoxy)ethoxy)acetate | 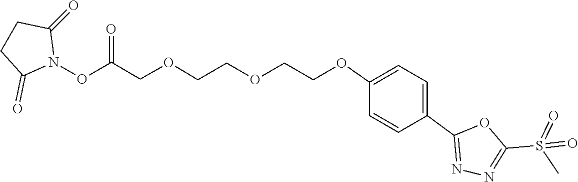 [SMILES: CS(=O)(=O)c1nnc(-c2ccc(OCCOCCOCC(=O)ON3C(=O)CCC3=O)cc2)o1] | 484 (M + H) |

### Preparation 10

### 2-(((2R,3R,4R,5R)-5-(2,4-Dioxo-3,4-dihydropyrimidin-1 (2H)-yl)-3-hydroxy-4-methoxytetrahydrofuran-2-yl)methyl)isoindoline-1,3-dione

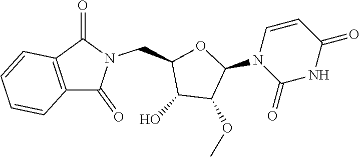 [SMILES: CO[C@@H]1[C@H](O)[C@@H](CN2C(=O)c3ccccc3C2=O)O[C@H]1n1ccc(=O)[nH]c1=O]

A solution of 1-((2R,3R,4R,5R)-4-hydroxy-5-(hydroxymethyl)-3-methoxytetrahydrofuran-2-yl)pyrimidine-2,4 (1H,3H)-dione (30 g, 120 mmol), isoindoline-1,3-dione (21 g, 140 mmol), DIAD (27 mL, 140 mmol), tributyl phosphine (36 mL, 150 mmol), and THF (300 mL) was stirred at ambient temperature for 12 h. The crude reaction was filtered, concentrated in vacuo, and purified via silica gel flash chromatography eluting with 0-100% EtOAc/hexanes to give the title compound as a white solid (6.0 g, 13%).

### Preparation 11

### 2-Cyanoethyl ((2R,3R,4R,5R)-5-(2,4-dioxo-3,4-dihydropyrimidin-1 (2H)-yl)-2-((1,3-dioxoisoindolin-2-yl)methyl)-4-methoxytetrahydrofuran-3-yl) diisopropylphosphoramidite

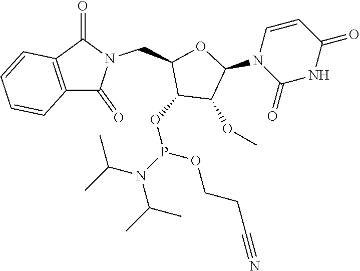 [SMILES: CO[C@@H]1[C@H](OP(OCCC#N)N(C(C)C)C(C)C)[C@@H](CN2C(=O)c3ccccc3C2=O)O[C@H]1n1ccc(=O)[nH]c1=O]

A solution of 2-(((2R,3R,4R,5R)-5-(2,4-dioxo-3,4-dihydropyrimidin-1 (2H)-yl)-3-hydroxy-4-methoxytetrahydrofuran-2-yl)methyl)isoindoline-1,3-dione (3.00 g, 7.74 mmol), 2-cyanoethyl-N,N-diisopropylchlorophosphoramidite (2.47 mL, 11.6 mmol), DIEA (4.05 mL, 23.2 mmol), and DCM (40 mL) was stirred at ambient temperature. After 1 hour, additional 2-cyanoethyl-N,N-diisopropylchlorophosphoramidite (0.82 mL, 3.8 mmol) was added. After 1 hour, the crude reaction was poured into a slurry of silica gel (15 g) in 30 mL of 1% TEA/DCM, concentrated in vacuo to a dry powder, and purified via silica gel flash chromatography eluting with 40-100% EtOAc/hexanes (0.5% TEA) to give the title compound as a white foam (3.70 g, 81%). ¹H NMR (d6-DMSO) d 11.4 (br s, 1H), 7.96-7.78 (m, 5H), 5.83 (dd, 1H), 5.71 (dd, 1H), 4.46-3.47 (m, 9H), 3.39 (s, 1.5H), 3.35 (s, 1.5H), 2.82-2.73 (m, 2H), 1.16-0.97 (m, 12H). ³¹P NMR (d6-DMSO) d 149.7, 149.4.

### Preparation 12

### 3′ Tetrazole Linker-Functionalized Sense Strand

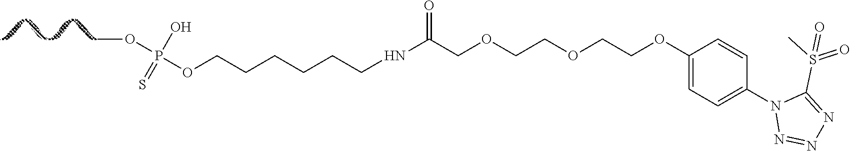 [SMILES: COP(O)(=S)OCCCCCCNC(=O)COCCOCCOc1ccc(-n2nnnc2S(C)(=O)=O)cc1]

To a solution of sodium bicarbonate (24 mg, 0.29 mmol) and SARM1-SSC6A (70 mg, 2.41 mL, 4.00 mM in water) was added a solution of 2,5-dioxopyrrolidin-1-yl 2-(2-(2-(4-(5-(methylsulfonyl)-1H-tetrazol-1-yl)phenoxy)ethoxy)ethoxy)acetate (1.2 mL, 400 mM in MeCN). Additional MeCN was added (1.2 mL) to bring the ratio of water:MeCN to 1:1 and the mixture was placed on a shaker at ambient temperature. After 1.5 h, analysis by LTQ showed that the reaction was complete. The contents of the tube were transferred to a 3,000 MWCO centrifugal spin filter, and the oligonucleotide was rinsed with 10×20 mL of RNAse-free water. The retentate was recovered. The procedure above was repeated in two trials using 70 and 35 mg of SARM1-SSC6A, and the recovered product lots were combined. The final product lot was 34 mL. Optical density measurement (A260) was 125.71, corresponding to a concentration of 590 μM, or 153 mg·ES/MS m/z 7638.5.

### Linker-SARM1 Duplex

MSPT-SARM1 sense strand (33 mL, 590 μM in water) and SARM1 antisense strand (7.047 mL, 3.45 mM in water) were mixed in a sample tube and vortexed for 10 s. UPLC indicated that the annealing was complete. The mixture was filtered through a 100K MWCO centrifugal filter and the filtrate was lyophilized. The lyophilized powder was reconstituted in 12 mL of water. The resulting solution was analyzed by optical density measurement (A260, 587), corresponding to a concentration of 1.592 mM, 292.76 mg. LTQ/MS m/z: 7686.3, 7638.3.

Conjugation of dsRNA to TfR Binding Proteins

Site-specific native or engineered cysteine amino acid residues in the TfR binding proteins were used to conjugate dsRNA. Cysteines can be engineered into the primary amino acid sequence of the TfR binding proteins. The approach of introducing cysteines as a means for conjugation has been described in WO 2018/232088, which is both incorporated by reference in its entirety and incorporated specifically in relation to conjugation via cysteine residues. For engineered cysteine conjugation, the TfR binding proteins were first reduced with 40 molar equivalents reducing agent dithiothreitol (DTT) at 37° C. for two hours, followed by desalting to remove reducing agent via dialysis or desalting columns. This is followed by re-oxidation of the TfR binding protein to reform the structural disulfides with 10 molar equivalent dehydroascorbic acid (DHAA) incubation at ambient temperature for two hours. A follow up desalting was performed to remove oxidizing agent.

Conjugation of dsRNA onto TfR binding proteins were done using the following methods.

### Conjugation Scheme

The conjugation method utilized the 3′SS tetrazole (MSPT)-functionalized dsRNA for conjugating onto the engineered cysteine of the TfR binding proteins. For this method, TfR binding protein was prepared similarly as above to make the engineered thiol available for conjugation by undergoing a reduction and oxidation process of the TfR binding proteins. This is followed by incubating the MSPT-dsRNA with the TfR binding proteins at 1.2 to 2 molar equivalents for overnight conjugation at ambient temperature.

TfR Binding Protein Conjugation with 3′ MSPT Linker

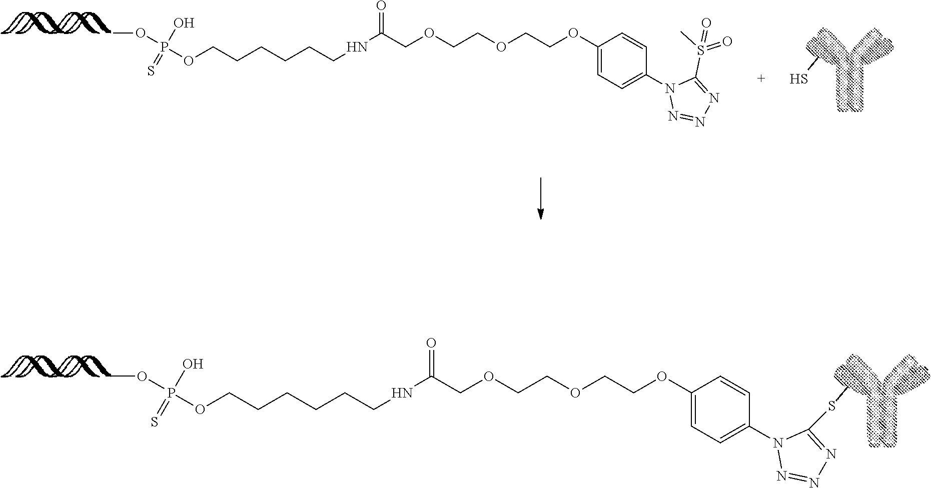

SMCC Linker-Functionalization of SARM1 ssRNA

To a solution of sodium bicarbonate (178 mg, 2.12 mmol) and SARM1 SS-3C6A (14.000 mL, 5.032 mM in water) was added a freshly prepared solution of (2,5-dioxopyrrolidin-1-yl) 4-[(2,5-dioxopyrrol-1-yl)methyl]cyclohexanecarboxylate (14.09 mL, 50 mM) in acetonitrile. The mixture was shaken at ambient temperature for 1.5 h. Analysis by LTQ showed that the reaction was complete. The mixture was acidified with 100 mL of 0.1 M NaH₂PO₄ buffer (pH 6) and filtered through a 0.22 μm filter and rinsed with water. The mixture was desalted via tangential flow filtration (2K MWCO membrane, Hydrosart), rinsing with 2 L of RNAse-free water. The retentate was recovered, frozen and lyophilized. The lyophilized powder was reconstituted in 31 mL RNAse-free water. The optical density measurement of the solution was 463.1, equating to 2.285 mM concentration and 519.27 mg total. Extinction coefficient was 202.68, LTQ/MS m/z 7331.6.

### SMCC-dsRNA Duplex

To a conical tube containing SS-SARM1-AMINO-SMCC, with appended C6-Amino-SMCC (12.05 mL, 0.016 mmol, 1.328 mmol/L), was added its corresponding SS-SARM1-ANTISENSE, with 5′-E-vinyl phosphonate, (0.0165 mmol, 2.619 mmol/L). The solutions were shaken at 25° C. for 30 minutes to give the desired SMCC-functionalized dsRNA (SMCC-dsRNA), then refrigerated to 10° C. for storage. The annealed solutions were sampled for LTQ purity and UPLC non-denaturing chromatography. Analysis via non-denaturing UPLC (run at 10° C.) shows a major single peak of 92% purity. LTQ-MS: (Antisense strand observed deconvoluted m/z=7768.4, calculated 7769.04; Sense strand observed deconvoluted m/z=7360.4, calculated mass 7361.17).

### Conjugation Scheme

The typical conjugation method utilized the SMCC-functionalized dsRNA for conjugating onto the engineered cysteine of the TfR binding proteins. For this method, TfR binding protein was prepared similarly as above to make the engineered thiol available for conjugation by undergoing a reduction and oxidation process of the TfR binding proteins. This is followed by incubating the SMCC-dsRNA at 1.2 molar equivalents with the TfR binding proteins for overnight conjugation at ambient temperature.

Optionally, following conjugation a maleimide hydrolysis step can be done to secure the linker-payload in terminal stage and avoid deconjugation during human body circulation via retro-Michael addition. This succinimide ring hydrolysis process was done by elevating the conjugate pH to 9.0 using 50 mM Arginine (stock solution of 0.7M arginine, pH 9.0 was used) and incubating the solution at 37° C. for 20 hours. The hydrolysis state of the maleimide was confirmed by LCMS characterization of +18 Da that is incurred by the water addition to the succinimide ring.

Step 1a: TfR Binding Protein Conjugation with SMCC Linker

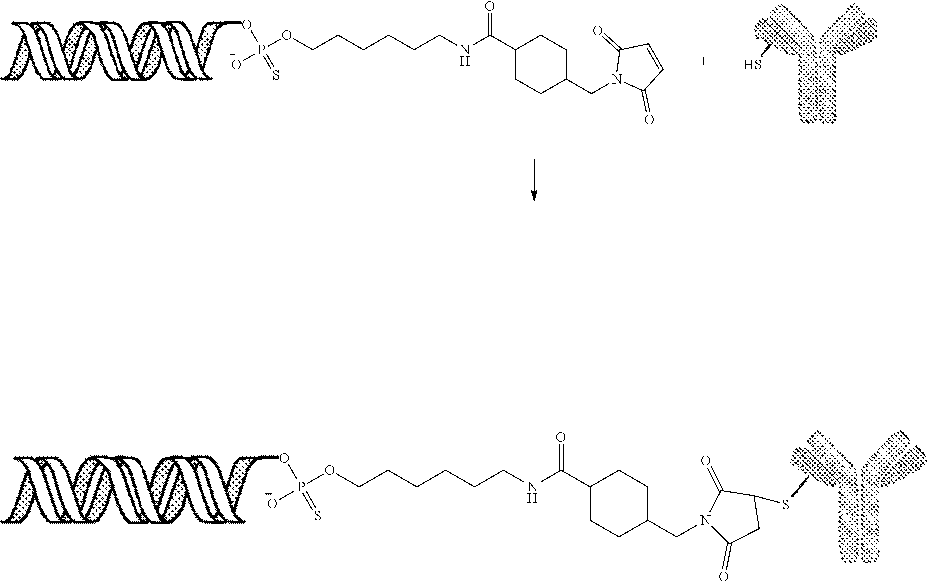

Step 1b: TfR Binding Protein Conjugation with SMCC Linker Ring Opening

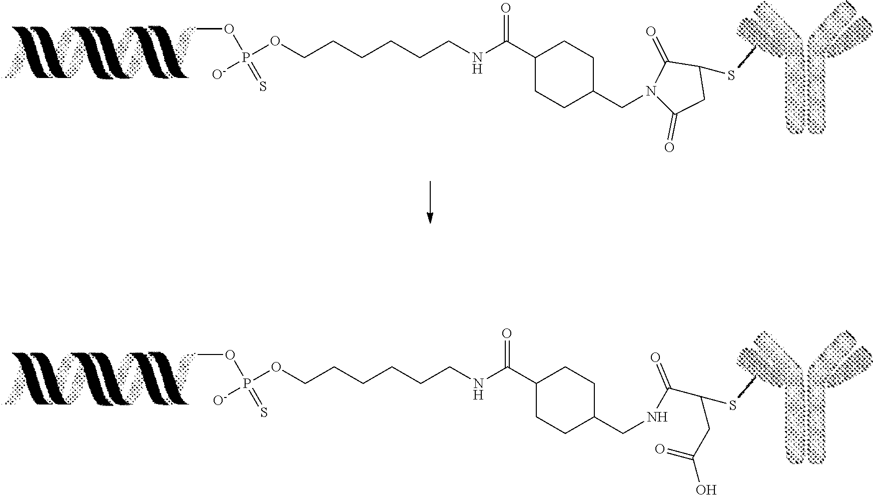 [SMILES: COP([O-])(=S)OCCCCCCNC(=O)C1CCC(CN2C(=O)CC(S)C2=O)CC1 ; COP([O-])(=S)OCCCCCCNC(=O)C1CCC(CNC(=O)C(S)CC(=O)O)CC1]

Conjugation was monitored using analytical anion exchange chromatography. A ProPac™ SAX-10 HPLC Column, 10 μm particle, 4 mm diameter, 250 mm length was utilized with the following method. Flow rate of 1 mL/min, Buffer A: 20 mM TRIS pH 7.0, Buffer B: 20 mM TRIS pH 7.0+1.5M NaCl, at 30° C.

Drug/siRNA to antibody/protein ratio (DAR) was calculated based on peak area % from the analytical anion exchange (aAEX) chromatogram.

Post conjugation of dsRNA to the TfR binding protein, excess dsRNA and unconjugated protein was removed by further purification. Either preparative size exclusion chromatography (SEC) or preparative anion exchange chromatography was utilized for purification of the final conjugate. Preparative SEC was performed using Cytiva Superdex® 200 in 1×PBS pH 7.2 under an isocratic condition. Alternatively, anion exchange, e.g., ThermoFisher POROS™ XQ, was used with starting buffer of 20 mM TRIS pH 7.0 and eluting with 20 column volume gradient with a buffer containing 20 mM TRIS pH 7.0 and 1M NaCl. These resulted in purified TfR binding protein-dsRNA conjugate devoid of excess dsRNA and minimal unconjugated protein. The resulting conjugate profile was analyzed by analytical anion exchange for final DAR quantitation (Table 16).

TABLE 16
siRNA/drug to TBP/antibody ratio (DAR)

| | Average | | | |
|---|---|---|---|---|
| | DAR | % of DAR0 | % of DAR1 | % of DAR2 |
| TBP5-SMCC- | 0.99 | 1.12 | 98.06 | 0.83 |
| dsRNA No. 8 | | | | |
| TBP5-SMCC- | 1 | 0 | 100 | 0 |
| dsRNA No. 7 | | | | |
| TBP5-SMCC- | 1 | 0 | 100 | 0 |
| dsRNA No. 6 | | | | |
| TBP5-MSPT- | 1 | 0.40 | 99.6 | 0 |
| dsRNA No. 8 | | | | |

### Example 4: In Vitro Characterization of SARM1 RNAi Agents

In vitro SARM1 knockdown of TBP5-SMCC-dsRNA No. 8 conjugate was assessed in SH-SH5Y cells and compared to the non-conjugated dsRNA No. 8 and cholesterol conjugated dsRNA No. 8.

SH-SY5Y Cell Culture and RNAi Treatment and Analysis: SH-SY5Y cells (ATCC CRL-2266) were derived from the SK-N-SH neuroblastoma cell line (Ross, R. A., et al., 1983. J Natl Cancer Inst 71, 741-747). The base medium was composed of a 1:1 mixture of ATCC-formulated Eagle's Minimum Essential Medium, (Cat No. 30-2003), and F12 Medium. The complete growth medium was supplemented with 10% fetal bovine serum, 1× amino acids, 1× sodium bicarbonate, and 1× penicillin-streptomycin (Gibco) and cells incubated at 37° C. in a humidified atmosphere of 5% CO₂. On Day One, SH-SY5Y cells were plated in 96 well fibronectin coated tissue culture plates and allowed to attach overnight. On Day Two, complete media was removed and replaced with RNAi agent in serum free media. Cells were incubated with RNAi agent for 72 hours, followed by media change with RNAi reagent for another 72 hours for a total of 144 hours of drug incubation before analysis of gene expression. Analysis of changes in gene expression in RNAi treated SH-SY5Y cells was measured using Cells-to-CT Kits following the manufacturer's protocol (ThermoFisher A35377). Predesigned gene expression assays (supplied as 20× mixtures) were selected from Applied Bio-systems (Foster City, CA, USA). The efficiencies of these assays (ThermoFisher Hs00240906_m1 SARM1 and ThermoFisher Hs99999905_m1 GAPDH) were characterized with a dilution series of cDNA. RT-QPCR was performed in MicroAmp Optical 384-well reaction plates using QuantStudio 7 Flex system. The delta-delta CT method of normalizing to the housekeeping gene GAPDH was used to determine relative amounts of gene expression. GraphPad Prism v9.0 was used to determine IC50 with a four parameter logistic fit.

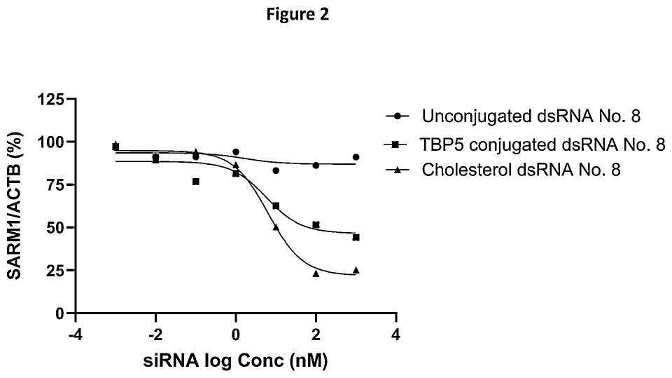

Results are shown in FIG. 2 and Table 17. IC50 potency of TBP5-SMCC-SARM1 dsRNA No. 8 conjugate was 5.79 nM, which is comparable to IC50 of cholesterol-dsRNA No. 8 conjugate. Maximal SARM1 knock down for TBP5-SMCC-dsRNA NO. 8 conjugate is 56%.

TABLE 17
In vitro activity of SARMI RNAi agents

| | | SH-SY5Y, 6 d |
|---|---|---|
| | SH-SY5Y, 6 d | % KD (knockdown) |
| SARM1 RNAi Agent | IC50 (nM) | of SARM1 at 1 μM |
| Unconjugated dsRNA No. 8 | N/A | 13% |
| TBP5-dsRNA No. 8 | 5.79 | 56% |
| Cholesterol-dsRNA No. 8 | 6.39 | 78% |

### Example 5. In Vivo Characterization of Human TBP-dsRNA Conjugates in Human TfR (hTfR) Transgenic Mice Against SARM1

To determine the efficacy of the human TfR binding proteins-dsRNA conjugates against SARM1, they were tested in human TfR transgenic knock-in mice where the extracellular domain of transferrin-receptor have been humanized. TBP5-SMCC-dsRNA No. 8 conjugate was dosed intravenously in hTfR transgenic mice at 10 mg/kg siRNA once or dosed four times weekly to compare against PBS dosed control group (n=5 per group). 28 days following initial dosing, mice were perfused under anesthesia and sacrificed, then hemibrain was collected and processed for assessment of gene expression changes using RT-qPCR.

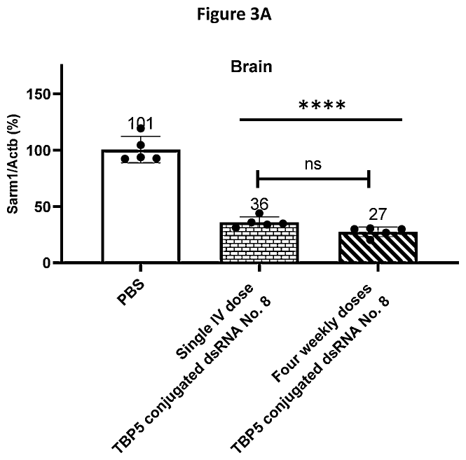

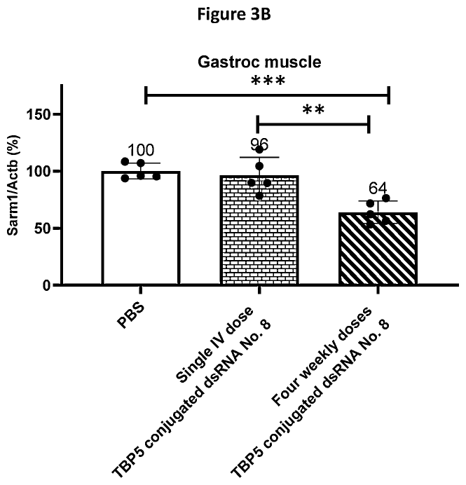

FIG. 3A shows significant reduction of SARM1 mRNA in the hemibrain in both dose groups of TBP5-SMCC-dsRNA No. 8, with a single dose demonstrating 64% reduction and four weekly dosing demonstrating 73% reduction from baseline PBS dosed group. Furthermore, assessment of gastrocnemius muscle demonstrated no reduction of SARM1 mRNA with a single dose, 36% reduction of SARM1 mRNA with four weekly dosing, compared to baseline PBS dosed group (FIG. 3B). Together, this data demonstrates TBP5-dsRNA No. 8 has higher activity in the brain than in the peripheral tissue.

### Example 6: In Vivo Characterization of the Human TfR Binding Proteins-dsRNA Conjugates

Following robust proof of concept demonstration of peripheral siRNA delivery into the CNS across BBB in mice, Pharmacodynamic properties of human TfR binding protein-SARM1 siRNA conjugates were assessed in non-human primates (NHPs) according to the following. Cynomolgus monkeys weighing 2-3 kg were dosed intravenously in the Saphenous vein in the thigh with i) PBS (n=4), ii) TBP5-dsRNA No. 7 (n=4) at 10 mg/kg effective siRNA concentration, or iii) TBP5-dsRNA No. 6 (n=4) at 10 mg/kg effective siRNA concentration and sacrificed 29 days after the first dose. Deeply anesthetized animals underwent cardiac perfusion, then brain, spinal cord and peripheral tissues were collected. The brain was coronally sectioned, 3 mm punches were collected from indicated subregions and frozen, as well as tissues were collected from spinal cord, liver and muscles to assess target mRNA levels by RT-qPCR in tissue homogenates. The total RNA from NHP tissues were isolated using the RNadvance Tissue kit (Beckman Coulter, Indianapolis, IN) manually or on a Biomek i7 liquid handler (Beckman Coulter), following the manufacturer's procedure with some modifications. In brief, the frozen tissue sections were mixed with one 5 mm stainless steel ball, lysis buffer and proteinase K, homogenized for 5 cycles of 30 seconds at 1200 rpm, with an interval of 20 seconds between cycles, on a 2010 GenoGrinder (SPEX SamplePrep, Metuchen, NJ). Tissues from some regions were shaved on dry ice, prior to homogenization. The homogenates were incubated at 37° C. for 1 hour, then extracted with an equal volume of phenol-chloroform. The RNA in the supernatant were purified with the RNadvance tissue kit, where a 30-minute digestion with DNase was included. The concentration and the purity (A260/A280) of the RNA elute were determined by spectrophotometry. RNA was normalized to 15 ng/10 μL PCR, digested again with ezDNase (ds-DNA specific) prior to reverse-transcription using the SSIV VILO kit (Thermo Fisher Scientific, Waltham, MA). The expression of the respective gene targets in the cDNA were determined using TaqMan qPCR assays on the QuantStudio 7 Pro platform (Thermo Fisher Scientific). Gene expression of SARM1 was normalized by GAPDH or ACTB using respective probes (ThermoFisher). The tissues analyzed and their acronyms include: LDRG, lumbar dorsal root ganglion; TSC, thoracic spinal cord; CSC, cervical spinal cord; LSC, lumbar spinal cord; LDRG, lumbar dorsal root ganglion; Medulla; Pons; Midbrain; MCTX, motor cortex.

To determine SARM1 protein levels, approximately 20 mg frozen sections of neural tissue biopsies were mixed with cold RIPA buffer (Pierce, Cat #89901, Thermo Scientific), containing the protease and phosphatase inhibitors (Halt™ Protease and Phosphatase Inhibitor Cocktail, Thermo Scientific), at a ratio of 20 mL buffer to 1 g tissue. The tissue-RIPA mixture was homogenized using a 5 mm stainless steel bead on a 2010 GenoGrinder (Spex SamplePrep). The homogenate was then centrifuged in a refrigerated centrifuge (Eppendorf, Hamburg, Germany), and the supernatant was transferred, made into multiple single-use aliquots, and stored in −80° C. for further analysis.

The protein concentration in the protein lysate was determined using the Pierce™ BCA Protein Assay Kit (Thermo Scientific), following manufacturer's instruction. In particular, the serially diluted BSA standards were analyzed in duplicate; while each protein lysate sample was diluted by 10 folds or by 20 folds in water, then analyzed in singlet, respectively. The protein concentration in the undiluted sample was then obtained by averaging that derived from the 10-fold diluted and that from the 20-fold diluted.

The level of SARM1 protein in the protein lysate was measured using an in-house developed MSD assay. Briefly, the MSD GOLD 96-well Small Spot Streptavidin SECTOR Plate (Meso Scale Diagnostics, Rockville, Maryland)) was simultaneously blocked with bovine serum albumin and coated with the capture antibody (biotinylated-mouse monoclonal anti-hSarm1, MAB7037, R&D Systems, Minneapolis, MN) with shaking at ambient temperature for 1 hour. After washing, the wells on each plate were incubated with the protein lysate or the recombinant hSARM1 protein (ab271737, Abcam, Waltham, MA) in the presence of MSD Blocker A (Meso Scale Diagnostics), with shaking at ambient temperature for 2 hours. The plates were washed again, then incubated with the detection antibody, the SULFO-TAG-conjugated mouse anti-hSARM1 monoclonal antibody (W16079A, BioLegend, San Diego, CA) in the presence of MSD Blocker A with shaking at ambient temperature for 1 hour. After the incubation, the plates were washed, then added with 2×MSD Read Buffer T (Meso Scale Diagnostics). The electrochemiluminescence signal was then measured on an MSD SQ120MM plate reader within 5 minutes of addition of the MSD Read Buffer.

To minimize variation, all biopsies from the same brain region, including those treated with the control article and those with the test article, as well as a set of serially diluted recombinant hSARM1 protein standards, were analyzed on the same MSD plate. All the samples, including the recombinant protein standards, were analyzed in duplicate. The DISCOVERY WORKBENCH software (Meso Scale Diagnostics) was used to analyze the raw electrochemiluminescence signal and calculate the corresponding protein concentration. Briefly, the raw electrochemiluminescence signal was first adjusted for the background. The standard curve on each MSD plate was then created by fitting the adjusted signal (Y-axis) and the SARM1 protein concentration (X-axis) in each of serially diluted SARM1 protein standards with the logistic 4P nonlinear regression model. The concentration of SARM1 protein in each diluted sample was then reversely calculated from respective adjusted signal, based on the standard curve. The level of SARM1 protein in each sample was normalized to the level of total protein, and the remaining SARM1 protein expression in the treated group was calculated as the percent of remaining SARM1 protein expression in the treatment group, relative to the average expression of that protein in the control article-treated control group.

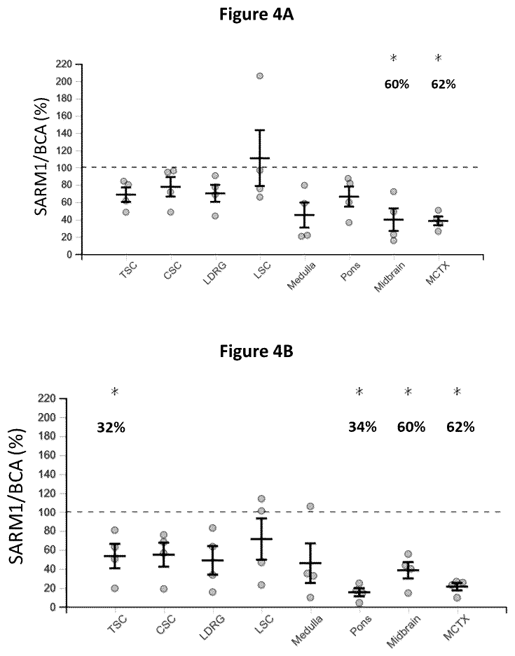

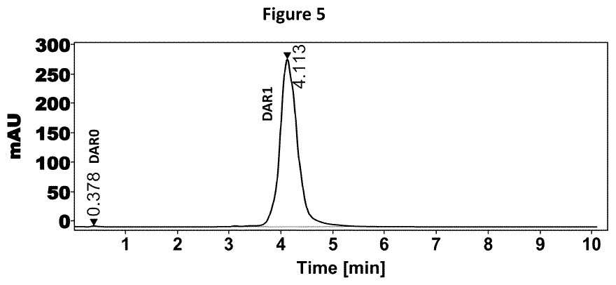

Single peripheral IV administration of TBP5-SARM1 siRNA (dsRNA No. 7 in Table 7) conjugate at 10 mg/kg dose in NHPs led to significant reduction of SARM1 protein in key brain and spinal cord regions compared to PBS treatment group at 29 days post dose. As shown in FIG. 4A, significant reduction of SARM1 protein was observed in Midbrain (60%) and motor cortex (62%). Single peripheral IV administration of TBP5-SARM1 siRNA (dsRNA No. 6 in Table 7) conjugate at 10 mg/kg dose in NHPs led to significant reduction of SARM1 protein in key brain and spinal cord regions compared to PBS treatment group at 29 days following dosing. As shown in FIG. 4B, significant reduction of SARM1 protein was observed in Thoracic Spinal Cord (32%), Pons (34%), Midbrain (60%), and motor cortex (62%).

### Example 7: In Vitro Characterization of SARM1 RNAi Agents

In vitro SARM1 knockdown by TBP5-SMCC-dsRNA No. 8 or TBP5-MSPT-dsRNA No. 8 conjugate was assessed in SH-SH5Y cells and compared.

SH-SY5Y Cell Culture and RNAi Treatment and Analysis: SH-SY5Y cells (ATCC CRL-2266) were derived from the SK-N-SH neuroblastoma cell line (Ross, R. A., et al., 1983. J Natl Cancer Inst 71, 741-747). The base medium was composed of a 1:1 mixture of ATCC-formulated Eagle's Minimum Essential Medium, (Cat No. 30-2003), and F12 Medium. The complete growth medium was supplemented with 10% fetal bovine serum, 1× amino acids, 1× sodium bicarbonate, and 1× penicillin-streptomycin (Gibco) and cells incubated at 37° C. in a humidified atmosphere of 5% CO₂. On Day One, SH-SY5Y cells were plated in 96 well fibronectin coated tissue culture plates and allowed to attach overnight. On Day Two, complete media was removed and replaced with RNAi agent in serum free media. Cells were incubated with RNAi agent for 72 hours, followed by media change with RNAi reagent for another 72 hours for a total of 144 hours of drug incubation before analysis of gene expression. Analysis of changes in gene expression in RNAi treated SH-SY5Y cells was measured using Cells-to-Cr Kits following the manufacturer's protocol (ThermoFisher A35377). Predesigned gene expression assays (supplied as 20× mixtures) were selected from Applied Bio-systems (Foster City, CA, USA). The efficiencies of these assays (ThermoFisher Hs00240906_m1 SARM1 and ThermoFisher Hs99999905_m1 GAPDH) were characterized with a dilution series of cDNA. RT-QPCR was performed in MicroAmp Optical 384-well reaction plates using QuantStudio 7 Flex system. The delta-delta CT method of normalizing to the housekeeping gene GAPDH was used to determine relative amounts of gene expression. GraphPad Prism v9.0 was used to determine IC50 with a four-parameter logistic fit.

Results are shown in Table 18. IC50 potency of TBP5-SMCC-SARM1 dsRNA No. 8 and TBP5-MSPT-SARM1 dsRNA No. 8 conjugates were 0.09 nM and 2.78 nM respectively. Maximal SARM1 knock down for TBP5-SMCC-dsRNA No. 8 and TBP5-MSPT-SARM1 dsRNA No. 8 conjugates were 41% and 59% respectively in SH-SY5Y cells.

TABLE 18
In vitro activity of SARMI RNAi agents

| | | SH-SY5Y, 6 d |
|---|---|---|
| | SH-SY5Y, 6 d | % KD (knockdown) |
| SARM1 RNAi Agent | IC50 (nM) | of SARM1 at 1 μM |
| TBP5-SMCC-dsRNA No. 8 | 0.09 | 41.03% |
| TBP5-MSPT-dsRNA No. 8 | 2.78 | 58.97% |

### Example 8. In Vivo Characterization of Human TBP-SARM1 dsRNA Conjugates with MSPT Linker in Human TfR (hTfR) Transgenic Mice

To determine the efficacy of the human TfR binding proteins-SARM1 dsRNA conjugates with MSPT linker, they were tested in human TfR transgenic knock-in mice where the extracellular domain of transferrin-receptor have been humanized. TBP5-SMCC-dsRNA No. 8 and TBP5-MSPT-dsRNA No. 8 conjugates were dosed intravenously in hTfR transgenic mice at 10 mg/kg siRNA once to compare against PBS dosed control group (n=3 per group). 28 days following initial dosing, mice were perfused under anesthesia and sacrificed, then hemibrain was collected and processed for assessment of gene expression changes using RT-qPCR.

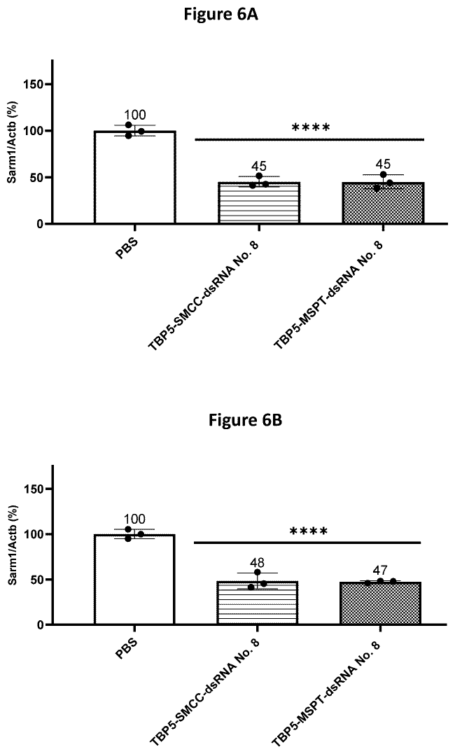

FIG. 6A shows significant reduction of SARM1 mRNA in the hemibrain in both dose groups of TBP5-SMCC-dsRNA No. 8 and TBP5-MSPT-dsRNA No. 8, with a single dose demonstrating 55% reduction from baseline PBS for both dosed groups. Furthermore, assessment of lumbar spinal cord demonstrated 52% and 53% reduction of SARM1 mRNA with a single dose of compared to baseline PBS dosed group with TBP5-SMCC-dsRNA No. 8 and TBP5-MSPT-dsRNA No. 8 respectively (FIG. 6B). Together, this data demonstrates TBP5-MSPT-dsRNA No. 8 has comparable activity is hemibrain and lumbar spinal cord with TBP5-SMCC-dsRNA No. 8.

SEQUENCE LISTING

| SEQ | | |
|---|---|---|
| ID | | |
| NO | Sequence | |
| 1 | SYSMN | |
| 2 | SISSSSSYIYYADSVKG | |
| 3 | RHGYSNSDAFDN | |
| 4 | RASQGISHYLV | |
| 5 | AASSLQS | |
| 6 | LQHNSYPWT | |
| 7 | EVQLVESGGGLVKPGGSLRLSCVASGFTFSSYSMNWVRQAPGKGLEWVSSISSSSSYIYYA | |
| | DSVKGRFTISRDNAKNSLYLQMNSLRAEDTAVYYCARRHGYSNSDAFDNWGQGTLVTVS | |
| | S | |
| 8 | DIQMTQSPSAMSASVGDRVTITCRASQGISHYLVWFQQKPGKVPKRLIYAASSLQSGVPSRF | |
| | SGSGSGTEFTLTISSLQPEDFATYYCLQHNSYPWTFGQGTKVEIK | |
| 9 | EVQLVESGGGLVKPGGSLRLSCVASGFTFSSYSMNWVRQAPGKGLEWVSSISSSSSYIYYA | |
| | DSVKGRFTISRDNAKNSLYLQMNSLRAEDTAVYYCARRHGYSNSDAFDNWGQGTLVTVS | |
| | SASTKGPSVFPLAPSSKSTSGGTAALGCLVKDYFPEPVTVSWNSGALTSGVHTFPAVLQSSG | |
| | LYSLSSVVTVPSSSLGTQTYICNVNHKPSNTKVDKRVEPKC | |
| 10 | DIQMTQSPSAMSASVGDRVTITCRASQGISHYLVWFQQKPGKVPKRLIYAASSLQSGVPSRF | |
| | SGSGSGTEFTLTISSLQPEDFATYYCLQHNSYPWTFGQGTKVEIKRTVAAPSVFIFPPSDEQL | |
| | KSGTASVVCLLNNFYPREAKVQWKVDNALQSGNSQESVTEQDSKDSTYSLSSTLTLSKAD | |
| | YEKHKVYACEVTHQGLSSPVTKSFNRGEC | |
| 11 | EVQLVESGGGLVKPGGSLRLSCVASGFTFSSYSMNWVRQAPGKGLEWVSSISSSSSYIYYA | |
| | DSVKGRFTISRDNAKNSLYLQMNSLRAEDTAVYYCARRHGYSNSDAFDNWGQGTLVTVS | |
| | SASTKGPCVFPLAPSSKSTSGGTAALGCLVKDYFPEPVTVSWNSGALTSGVHTFPAVLQSSG | |
| | LYSLSSVVTVPSSSLGTQTYICNVNHKPSNTKVDKRVEPKCDKTHTGGGGQGGGGQGGGG | |
| | QGGGGQGGGGQEVQLLESGGGLVQPGGSLRLSCAASGRYIDETAVAWFRQAPGKGREFV | |
| | AGIGGGVDITYYADSVKGRFTISRDNSKNTLYLQMNSLRPEDTAVYYCGARPGRPLITSKV | |
| | ADLYPYWGQGTLVTVSSPP | |
| 12 | DIQMTQSPSAMSASVGDRVTITCRASQGISHYLVWFQQKPGKVPKRLIYAASSLQSGVPSRF | |
| | SGSGSGTEFTLTISSLQPEDFATYYCLQHNSYPWTFGQGTKVEIKRTVAAPSVFIFPPSDEQL | |
| | KSGTASVVCLLNNFYPREAKVQWKVDNALQCGNSQESVTEQDSKDSTYSLSSTLTLSKAD | |
| | YEKHKVYACEVTHQGLSSPVTKSFNRGEC | |
| 13 | EVQLVESGGGLVKPGGSLRLSCVASGFTFSSYSMNWVRQAPGKGLEWVSSISSSSSYIYYA | |
| | DSVKGRFTISRDNAKNSLYLQMNSLRAEDTAVYYCARRHGYSNSDAFDNWGQGTLVTVS | |
| | SASTKGPXVFPLAPCSRSTSESTAALGCLVKDYFPEPVTVSWNSGALTSGVHTFPAVLQSSG | |
| | LYSLSSVVTVPSSSLGTKTYTCNVDHKPSNTKVDKRVESKYGPPCPPCPAPEAAGGPSVFLF | |
| | PPKPKDTLMISRTPEVTCVVVDVSQEDPEVQFNWYVDGVEVHNAKTKPREEQFNSTYRVV | |
| | SVLTVLHQDWLNGKEYKCKVSNKGLPSSIEKTISKAKGQPREPQVYTLPPSQEEMTKNQVS | |
| | LTCLVKGFYPSDIAVEWESNGQPENNYKTTPPVLDSDGSFFLYSRLTVDKSRWQEGNVFSC | |
| | SVMHEALHNHYTQKSLSLSLG, wherein X is S or C. | |
| 14 | EVQLVESGGGLVKPGGSLRLSCVASGFTFSSYSMNWVRQAPGKGLEWVSSISSSSSYIYYA | |
| | DSVKGRFTISRDNAKNSLYLQMNSLRAEDTAVYYCARRHGYSNSDAFDNWGQGTLVTVS | |
| | SASTKGPSVFPLAPCSRSTSESTAALGCLVKDYFPEPVTVSWNSGALTSGVHTFPAVLQSSG | |
| | LYSLSSVVTVPSSSLGTKTYTCNVDHKPSNTKVDKRVESKYGPPCPPCPAPEAAGGPSVFLF | |
| | PPKPKDTLMISRTPEVTCVVVDVSQEDPEVQFNWYVDGVEVHNAKTKPREEQFNSTYRVV | |
| | SVLTVLHQDWLNGKEYKCKVSNKGLPSSIEKTISKAKGQPREPQVSTLPPSQEEMTKNQVS | |
| | LMCLVYGFYPSDIXVEWESNGQPENNYKTTPPVLDSDGSFFLYSVLTVDKSRWQEGNVFSC | |
| | SVMHEALHNHYTQKSLSLSLG, wherein X is A or C. | |
| 15 | ESKYGPPCPPCPAPEAAGGPSVFLFPPKPKDTLMISRTPEVTCVVVDVSQEDPEVQFNWYVD | |
| | GVEVHNAKTKPREEQFNSTYRVVSVLTVLHQDWLNGKEYKCKVSNKGLPSSIEKTISKAK | |
| | GQPREPQVYTLPPSQGDMTKNQVQLTCLVKGFYPSDIXVEWESNGQPENNYKTTPPVLDSD | |
| | GSFFLASRLTVDKSRWQEGNVFSCSVMHEALHNHYTQKSLSLSLG, wherein X is A or C. | |
| 16 | EVQLVESGGGLVKPGGSLRLSCVASGFTFSSYSMNWVRQAPGKGLEWVSSISSSSSYIYYA | |
| | DSVKGRFTISRDNAKNSLYLQMNSLRAEDTAVYYCARRHGYSNSDAFDNWGQGTLVTVS | |
| | SASTKGPCVFPLAPCSRSTSESTAALGCLVKDYFPEPVTVSWNSGALTSGVHTFPAVLQSSG | |
| | LYSLSSVVTVPSSSLGTKTYTCNVDHKPSNTKVDKRVESKYGPPCPPCPAPEAAGGPSVFLF | |
| | PPKPKDTLMISRTPEVTCVVVDVSQEDPEVQFNWYVDGVEVHNAKTKPREEQFNSTYRVV | |
| | SVLTVLHQDWLNGKEYKCKVSNKGLPSSIEKTISKAKGQPREPQVSTLPPSQEEMTKNQVS | |
| | LMCLVYGFYPSDIAVEWESNGQPENNYKTTPPVLDSDGSFFLYSVLTVDKSRWQEGNVFSC | |
| | SVMHEALHNHYTQKSLSLSLG | |
| 17 | ESKYGPPCPPCPAPEAAGGPSVFLFPPKPKDTLMISRTPEVTCVVVDVSQEDPEVQFNWYVD | |
| | GVEVHNAKTKPREEQFNSTYRVVSVLTVLHQDWLNGKEYKCKVSNKGLPSSIEKTISKAK | |
| | GQPREPQVYTLPPSQGDMTKNQVQLTCLVKGFYPSDIAVEWESNGQPENNYKTTPPVLDSD | |
| | GSFFLASRLTVDKSRWQEGNVFSCSVMHEALHNHYTQKSLSLSLG | |
| 18 | QVQLVQSGAEVKKPGSSVKVSCKASGYTFSSYAIEWVRQAPGQGLEWMGGILPGSGTINY | |
| | NEKFKGRVTITADKSTSTAYMELSSLRSEDTAVYYCARMSSNSDQGFDLWGQGTLVTVSS | |
| | ASTKGPXVFPLAPCSRSTSESTAALGCLVKDYFPEPVTVSWNSGALTSGVHTFPAVLQSSGL | |
| | YSLSSVVTVPSSSLGTKTYTCNVDHKPSNTKVDKRVESKYGPPCPPCPAPEAAGGPSVFLFP | |
| | PKPKDTLMISRTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPSQEEMTKNQVSL | |
| | TCLVKGFYPSDIAVEWESNGQPENNYKTTPPVLDSDGSFLLYSKLTVDKSRWQEGNVFSCS | |
| | VMHEALHNHYTQKSLSLSLG, wherein X is S or C. | |
| 19 | DIQMTQSPSSLSASVGDRVTITCKASQGISRFLSWFQQKPGKAPKSLIYAVSSLVDGVPSRFS | |
| | GSGSGTDFTLTISSLQPEDFATYYCVQYNSYPYGFGGGTKVEIKRTVAAPSVFIFPPSDEQLK | |
| | SGTASVVCLLNNFYPREAKVQWKVDNALQSGNSQESVTEQDSKDSTYSLSSTLTLSKADYE | |
| | KHKVYACEVTHQGLSSPVTKSFNRGEC | |
| 20 | ETAVA | |
| 21 | GIGGGVDITYYADSVKG | |
| 22 | RPGRPLITSKVADLYPY | |
| 23 | EVQLLESGGGLVQPGGSLRLSCAASGRYIDETAVAWFRQAPGKGREFVAGIGGGVDITYYA | |
| | DSVKGRFTISRDNSKNTLYLQMNSLRPEDTAVYYCGARPGRPLITSKVADLYPYWGQGTLV | |
| | TVSSPP | |
| 24 | GGGGQGGGGQGGGGQGGGGQ | |
| 25 | GSYWIC | |
| 26 | CIYSTSGGRTYYASWVKG | |
| 27 | GDDSISDAYFDL | |
| 28 | QSSQSVYNNNRLA | |
| 29 | DASTLAS | |
| 30 | QGTYFSSGWSWA | |
| 31 | QSLEESGGDLVKPEGSLTLTCTASGFSFSGSYWICWVRQAPGKGLEWIGCIYSTSGGRTYYA | |
| | SWVKGRFTISKTSSTTVTLQMTSLTAADTATYFCARGDDSISDAYFDLWGPGTLVTVSS | |
| 32 | ALDMTQTASPVSAAVGGTVTINCQSSQSVYNNNRLAWYQQKPGQPPKLLIYDASTLASGV | |
| | PSRFKGSGSGTQFTLTISGVQSDDSATYYCQGTYFSSGWSWAFGGGTEVVVK | |
| 33 | QSLEESGGDLVKPEGSLTLTCTASGFSFSGSYWICWVRQAPGKGLEWIGCIYSTSGGRTYYA | |
| | SWVKGRFTISKTSSTTVTLQMTSLTAADTATYFCARGDDSISDAYFDLWGPGTLVTVSSAST | |
| | KGPCVFPLAPCSRSTSESTAALGCLVKDYFPEPVTVSWNSGALTSGVHTFPAVLQSSGLYSL | |
| | SSVVTVPSSSLGTKTYTCNVDHKPSNTKVDKRVESKYGPPCPPCPAPEAAGGPSVFLFPPKP | |
| | KDTLMISRTPEVTCVVVDVSQEDPEVQFNWYVDGVEVHNAKTKPREEQFNSTYRVVSVLT | |
| | VLHQDWLNGKEYKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQEGNVFSCSVM | |
| | HEALHNHYTQKSLSLSLG | |
| 34 | ALDMTQTASPVSAAVGGTVTINCQSSQSVYNNNRLAWYQQKPGQPPKLLIYDASTLASGV | |
| | PSRFKGSGSGTQFTLTISGVQSDDSATYYCQGTYFSSGWSWAFGGGTEVVVKRTVAAPSVF | |
| | IFPPSDEQLKSGTASVVCLLNNFYPREAKVQWKVDNALQSGNSQESVTEQDSKDSTYSLSS | |
| | TLTLSKADYEKHKVYACEVTHQGLSSPVTKSFNRGEC | |
| 35 | GUUGCUCGACUCUAACCGCUA | |
| 36 | UAGCGGUUAGAGUCGAGCAACGG | |
| 37 | UUCGCCAACUAUUCUACGUGA | |
| 38 | UCACGUAGAAUAGUUGGCGAAGG | |
| 39 | ACCUUCGCCAACUAUUCUACA | |
| 40 | UGUAGAAUAGUUGGCGAAGGUCU | |
| 41 | CCGCAAGAGGUUCUUUAGGGA | |
| 42 | UCCCUAAAGAACCUCUUGCGGGU | |
| 43 | mG*mU*mUmGmCmUmCmGfAfCfUmCmUmAmAmCmCmGmC*mU*mA | |
| 44 | mU*fA*mGmCfGmGmUfUmAmGmAmGmUfCmGfAmGmCmAmAmC*mG*mG | |
| 45 | mU*mU*mCmGmCmCmAmAfCfUfAmUmUmCmUmAmCmGmU*mG*mA | |
| 46 | mU*fC*mAmCfGmUmAfGmAmAmUmAmGfUmUfGmGmCmGmAmA*mG*mG | |
| 47 | mA*mC*mCmUmUmCmGmCfCfAfAmCmUmAmUmUmCmUmA*mC*mA | |
| 48 | mU*fG*mUmAfGmAmAfUmAmGmUmUmGfGmCfGmAmAmGmGmU*mC*mU | |
| 49 | mC*mC*mGmCmAmAmGmAfGfGfUmUmCmUmUmUmAmGmG*mG*mA | |
| 50 | mU*fC*mCmCfUmAmAfAmGmAmAmCmCfUmCfUmUmGmCmGmG*mG*mU | |
| 51 | mU*fC*mAmCfGmUfAmGmAmAmUmAmGfUmUfGmGmCmGmAmA*mG*mG | |
| 52 | mU*fC*fAmCmGmUfAmGmAmAmUmAmGfUmUfGmGmCmGmAmA*mG*mG | |
| 53 | mU*fC*mAmCmGmUmAmGmAmAmUmAmGfUmUmGmGmCmGmAmA*mG*mG | |
| 54 | mU*fA*mGmCfGmGfUmUmAmGmAmGmUfCmGfAmGmCmAmAmC*mG*mG | |
| 55 | mU*fA*fGmCmGmGfUmUmAmGmAmGmUfCmGfAmGmCmAmAmC*mG*mG | |
| 56 | mU*fA*mGmCmGmGmUmUmAmGmAmGmUfCmGmAmGmCmAmAmC*mG*mG | |
| 57 | mU*fC*mCmCfUmAfAmAmGmAmAmCmCfUmCfUmUmGmCmGmG*mG*mU | |
| 58 | mU*fC*fCmCmUmAfAmAmGmAmAmCmCfUmCfUmUmGmCmGmG*mG*mU | |
| 59 | mU*fC*mCmCmUmAmAmAmGmAmAmCmCfUmCmUmUmGmCmGmG*mG*mU | |
| 60 | mU*fA*mGmCmGfGmUmUmAmGmAmGmUfCmGfAmGmCmAmAmC*mG*mG | |
| 61 | mU*fC*mAmCmGfUmAmGmAmAmUmAmGfUmUfGmGmCmGmAmA*mG*mG | |
| 62 | mU*fG*mUmAmGfAmAmUmAmGmUmUmGfGmCfGmAmAmGmGmU*mC*mU | |
| 63 | mU*fC*mCmCmUfAmAmAmGmAmAmCmCfUmCfUmUmGmCmGmG*mG*mU | |
| 64 | 1 | ATCTCCCAGC TCAGCCGAGC CCGTGCCCAG GCCACGCTTT GTTCCAGCCG CCGCCTCCTC |
| | 61 | TACCCTACGG CGTCCGGAGC CATCCCTCGC CTGCTCGCTC TCTCCTTTCG CCCACTCCCT |
| | 121 | GCATCTGGGC CTGCATCACC TTTGCCAACC GCTCCCCCGA TCCTGCCGAC ACTCCTCCCC |
| | 181 | CAAACTTCTG ACCGGCACCC TTGCCTGGTA CCCTTCTCTC CATTCCTCCC CCTCCATCTT |
| | 241 | CTTTCCCCGA CCCCTCTCGG GTCCCTCTTT TCCCAAAACC CGGGTCTCTC CGCGTGGCCC |
| | 301 | CGCCTCCAGG CCGGGGATGT CCCCCGCGGC CCCGCGCCCA TGGTCCTGAC GCTGCTTCTC |
| | 361 | TCCGCCTACA AGCTGTGTCG CTTCTTCGCC ATGTCGGGCC CACGGCCGGG CGCCGAGCGG |
| | 421 | CTGGCGGTGC CTGGGCCAGA TGGGGGCGGT GGCACGGGCC CATGGTGGGC TGCGGGTGGC |
| | 481 | CGCGGGCCCC GCGAAGTGTC GCCGGGGGCA GGCACCGAGG TGCAGGACGC CCTGGAGCGC |
| | 541 | GCGCTGCCGG AGCTGCAGCA GGCCTTGTCC GCGCTGAAGC AGGCGGGCGG CGCGCGGGCC |
| | 601 | GTGGGCGCCG GCCTGGCCGA GGTCTTCCAA CTGGTGGAGG AGGCCTGGCT GCTGCCGGCC |
| | 661 | GTGGGCCGCG AGGTAGCCCA GGGTCTGTGC GACGCCATCC GCCTCGATGG CGGCCTCGAC |
| | 721 | CTGCTGTTGC GGCTGCTGCA GGCGCCGGAG TTGGAGACGC GTGTGCAGGC CGCGCGCCTG |
| | 781 | CTGGAGCAGA TCCTGGTGGC TGAGAACCGA GACCGCGTGG CGCGCATTGG GCTGGGCGTG |
| | 841 | ATCCTGAACC TGGCGAAGGA ACGCGAACCC GTAGAGCTGG CGCGGAGCGT GGCAGGCATC |
| | 901 | TTGGAGCACA TGTTCAAGCA TTCGGAGGAG ACATGCCAGA GGCTGGTGGC GGCCGGCGGC |
| | 961 | CTGGACGCGG TGCTGTATTG GTGCCGCCGC ACGGACCCCG CGCTGCTGCG CCACTGCGCG |
| | 1021 | CTGGCGCTGG GCAACTGCGC GCTGCACGGG GGCCAGGCGG TGCAGCGACG CATGGTAGAG |
| | 1081 | AAGCGCGCAG CCGAGTGGCT CTTCCCGCTC GCCTTCTCCA AGGAGGACGA GCTGCTTCGG |
| | 1141 | CTGCACGCCT GCCTCGCAGT AGCGGTGTTG GCGACTAACA AGGAGGTGGA GCGCGAGGTG |
| | 1201 | GAGCGCTCGG GCACGCTGGC GCTCGTGGAG CCGCTTGTGG CCTCGCTGGA CCCTGGCCGC |
| | 1261 | TTCGCCCGCT GTCTGGTGGA CGCCAGCGAC ACAAGCCAGG GCCGCGGGCC CGACGACCTG |
| | 1321 | CAGCGCCTCG TGCCGTTGCT CGACTCTAAC CGCTTGGAGG CGCAGTGCAT CGGGGCTTTC |
| | 1381 | TACCTCTGCG CCGAGGCTGC CATCAAGAGC CTGCAAGGCA AGACCAAGGT GTTCAGCGAC |
| | 1441 | ATCGGCGCCA TCCAGAGCCT GAAACGCCTG GTTTCCTACT CTACCAATGG CACTAAGTCG |
| | 1501 | GCGCTGGCCA AGCGCGCGCT GCGCCTGCTG GGCGAGGAGG TGCCACGGCC CATCCTGCCC |
| | 1561 | TCCGTGCCCA GCTGGAAGGA GGCCGAGGTT CAGACGTGGC TGCAGCAGAT CGGTTTCTCC |
| | 1621 | AAGTACTGCG AGAGCTTCCG GGAGCAGCAG GTGGATGGCG ACCTGCTTCT GCGGCTCACG |
| | 1681 | GAGGAGGAAC TCCAGACCGA CCTGGGCATG AAATCGGGCA TCACCCGCAA GAGGTTCTTT |
| | 1741 | AGGGAGCTCA CGGAGCTCAA GACCTTCGCC AACTATTCTA CGTGCGACCG CAGCAACCTG |
| | 1801 | GCGGACTGGC TGGGCAGCCT GGACCCGCGC TTCCGCCAGT ACACCTACGG CCTGGTCAGC |
| | 1861 | TGCGGCCTGG ACCGCTCCCT GCTGCACCGC GTGTCTGAGC AGCAGCTGCT GGAAGACTGC |
| | 1921 | GGCATCCACC TGGGCGTGCA CCGCGCCCGC ATCCTCACGG CGGCCAGAGA AATGCTACAC |
| | 1981 | TCCCCGCTGC CCTGTACTGG TGGCAAACCC AGTGGGGACA CTCCAGATGT CTTCATCAGC |
| | 2041 | TACCGCCGGA ACTCAGGTTC CCAGCTGGCC AGTCTCCTGA AGGTGCACCT GCAGCTGCAT |
| | 2101 | GGCTTCAGTG TCTTCATTGA TGTGGAGAAG CTGGAAGCAG GCAAGTTCGA GGACAAACTC |
| | 2161 | ATCCAGAGTG TCATGGGTGC CCGCAACTTT GTGTTGGTGC TATCACCTGG AGCACTGGAC |
| | 2221 | AAGTGCATGC AAGACCATGA CTGCAAGGAT TGGGTGCATA AGGAGATTGT GACTGCTTTA |
| | 2281 | AGCTGCGGCA AGAACATTGT GCCCATCATT GATGGCTTCG AGTGGCCTGA GCCCCAGGTC |
| | 2341 | CTGCCTGAGG ACATGCAGGC TGTGCTTACT TTCAACGGTA TCAAGTGGTC CCACGAATAC |
| | 2401 | CAGGAGGCCA CCATTGAGAA GATCATCCGC TTCCTGCAGG GCCGCTCCTC CCGGGACTCA |
| | 2461 | TCTGCAGGCT CTGACACCAG TTTGGAGGGT GCTGCACCCA TGGGTCCAAC CTAACCAGTC |
| | 2521 | CCCAGTTCCC CAGCCCTGCT GTGACTTCCA TTTCCATCGT CCTTTCTGAA GGAACAGCTC |
| | 2581 | CTGAAACCAG TCTCCCTGGG CTGAGACAAC CTGGGCTCTT CTTAGGAAAT GGCTCTCCCT |
| | 2641 | CCCCCTGTCC CCCACCCTCA TGGCCCACCT CCAACCCACT TTCCTCAGTA TCTGGAGAGG |
| | 2701 | GAAGGGAAGT CAGGCTTGGG CACGGGAGGT TAGAACTCCC CCAGGCCCTG CCATTGGGTT |
| | 2761 | GTCTGTCTCC GTCATGGGGA GGGTCCCTGC TCAGTTCTGG AGACACTGGA GTTGGGGTGG |
| | 2821 | GGGTGGTTCT GCATTCCCTT CTCCTGCTGA TAGCAGTCAG CTTGAGGAGG ATGACGGAAG |
| | 2881 | GCAGCCTCAG ACAGGAATTA AGGCAATGCC CAGGCGGGCC TGGGCACTGT ATTCTGAGCA |
| | 2941 | AGGGCCTGGG CCCAGGAGCC AGCCAGGGAT GAGTGCCATC ATGGCTCTCC ACTCAGACTG |
| | 3001 | TGCCTGGCCC CTGCACTTAC AACTTCCTGC CGCTCTGTGG CCTTGCCCTG TAATCACTCA |
| | 3061 | GTGCCCTTAG CTAGCCTGAC TAAGTCCCAG ATCCCCTACA GCTTCCTTCG GTGTGGTATC |
| | 3121 | TTTTGCCACA TCCAGGGCGA GGGTTGAGGC AAACCAGCCC TCCCTCTGAC TTCCTTGTCA |
| | 3181 | CTGCAGCCAG CTTTGCTGCA CTTGCTGGTG CACAGGAGCC TCCTGTTTGG GCCTGGGTCT |
| | 3241 | GGGCATGGGG AGGCCGTGCC TCAAAGCCCA CCCTACCCCA TGCCTTGGTG CTGTGCCTCA |
| | 3301 | GGCTCCTTCC TGGTCTGGCC CAGCTGGCTT CCCCAGCCCC TCAGCCATCC AGGGCTACCC |
| | 3361 | ACTGCTTACT CAGGGACCAG GCAGCCCCCA TGGCAGTAAA AGCAGCCTAG ACAGAACCTG |
| | 3421 | CAGCTCTGTG GAAAGAGGCA AAGTCCTGAA AAGGCAAAGG GTTGTCACTT AGGGCAGCTT |
| | 3481 | CTCCAACTTT AACATGCATC CAAGTCACCT GGGAATGTTG TTAAAATCAG GAGATCTGGG |
| | 3541 | GTGGGGCCTA GGACTCTGCA TTTCTTACAG ATTCCCAGGT GAGCTGATGC TGGTGGTTAA |
| | 3601 | GGGTAGCAAA TCTCTAAAGC ACGAAGCCCT CACAAATCTT TGCCATTTCC CAAACACTCC |
| | 3661 | GCTCCATGGT CTCCAGTCAT CAGAGCAACT CTACCTGGTA TTATCATCCC CATTTTACAG |
| | 3721 | ATAATGACAC TGAGGCTCAG AAAGGTTGAG GATAAGCCCA CTTTCCTGTC ATTAGTGGCA |
| | 3781 | GCCCCAGATC CAGACCTAGG CCTCCTGGCA CCCAGTCCAC TGGCAGTGGA ATTGCTTTCC |
| | 3841 | TGAGAATCAT TCTGAGGCTG GGCTATTGCT TCTCCCTTGC TTCAAAGAAT CTAGCAGCGG |
| | 3901 | GGGATAGGAT TTTGCAACAA AAAGCTGACC CAGAGGCCAT ACAGAGCAGG AATATCCCAT |
| | 3961 | TGCCCCCTCC TCCACTGGGT TCAGAGGGTA AGAAAGCACC CTCCAATAAA CCCAGGCTCC |
| | 4021 | AGGCCGTGGG GGCTGCTGAA GGCTCTTTCC CCGCAAGGGC CAGGTGTTGA CACCTTAAAG |
| | 4081 | CTGGCTGCGC CCCCAGCCCC ACTCTTGGCT GTGCTGGCCA GGTGACTCCT AGTTCTTGGC |
| | 4141 | CACATCATCA GAAAGTCAAA GGTCTCACTC CAGGTTTGGG GCTCCTTCCT TCCACTCCCC |
| | 4201 | TCCCTGCCAG AGTCTGTCTT GGCCAGTGCC AGCCTCGATG CTTTGGTTTT GACCCCACCT |
| | 4261 | GATCCTCCTT TCCTCATGCA GCACAAGTGC TCACCGGGGC CAGAGCCAGG GCATGGATAT |
| | 4321 | GACAAGCAGG GCAGCCTGGA CACTGCCCTC ACAGGACAGC GCCAATAACA ATACAGTGTC |
| | 4381 | TGAGTATCTC CAGGGGATGA TTTCTGGCTC TTTGTCTCCA ATCAGTCCCA CTCCCTCCTG |
| | 4441 | AGGTCCCCAA GGGCAGTATT CAGAGAGGTT TCCTGCGTTT TATTTCTATT TGGTATACCC |
| | 4501 | TCCACTGTTG TCCACTGCCC TGTGTGGCCT TCTGGTTGAC CTCTGCCCGA TCTTCTGTCT |
| | 4561 | CTCTGAGGGA ATCAGAGTCC AGCATCCAGC CCCAGCTGGA ACAGCTGAAG TCACAAGCCT |
| | 4621 | CCTCTAAGCC AAGGCCAGTG TGTTCAGAGG TGACTGCCAC CCATACTAGG ACAAACACAG |
| | 4681 | CTCAGATCAC CAGGTCAAGC ACCTAGGCCT GGCTTCTCCT GAGACAGAGG ACTCAGAAGT |
| | 4741 | GGCCTTTCCT CCAAAGCCTG CTCAGACACA GGTCTGTAGG GCCAGGGTGT TCTGCTTGGC |
| | 4801 | TGGGCTGCAG CTGCTACCCC TCGGTTGGGG CTGAGTCAGC CAGATCCTCC CCCTACTTCT |
| | 4861 | CCCCAAGGGC CAAGAACTGC TCAGGGACAT TAAAGGTCAA AAGTCCAGCC ACACTCATTC |
| | 4921 | ATCCTTTCCC CAGGCCCATG AAGAGAGGCA TCTCATTGTA GAATGTATGA GGAAGTGGGA |
| | 4981 | AGTATCTCAG AGAATCAGCT AAGTTTCCTA ACTTGTCCAT CCAAATGTGA TCACCACGAT |
| | 5041 | TCAACAATTT GGGGCATTGC TGATCTAGCC GTTCCTAGTG GGGCTTGCTC AAGGTTGCAC |
| | 5101 | AGCGAGTCAG TAGAAGCCCT GGCTGGCCCC ACTTGGTACC AATCCACCAG GCAGCTCAGG |
| | 5161 | GCTCCTGCCC AGCCCAGCAG CTTCTGTTGT CTAACGTATG GCAGGCAGAC TGGGAGCAGG |
| | 5221 | AAAACAGAGG GCCCCAAAGC CCAAGGCACC AGAAGGTTTG TTTCAGTTTG CTGAAGCTGA |
| | 5281 | TTTGTAATGA TTGGCACTCT TCAGCCAGGG GAGTGGGTAG GCCATAGCCA AGGATCGATT |
| | 5341 | CCCCAACCAC AGCAAAGGCA ACACTCTTCC TCCAGAGATC ACCAAGCCCC TCTTACCTCC |
| | 5401 | CTCCCTCCTT CCCAAGGCTG GCACTAACCA GGTACCACAT TCATTGTTAA GGAATGGCTG |
| | 5461 | ATGACTGCTA CACGTGTTGG GAACCTGGTT GGGGCTGTGC AGTTTGGGCT GGAAGGAGAG |
| | 5521 | ATGCCAGCCC TCGTGCTGCC TCTGGTCCCT GAAGTGTCAC CTCTCTCAGG ACCTCTCCTC |
| | 5581 | TGGCCTGTGG GGTTATAAGT GATGGATAGC AGAAAGGGAG AACTGACTCC TGTCCCAAAT |
| | 5641 | AGCTCCTCTG CCACCTGTCC TGCAGTGGGC CTGTGTGGGT TATGATTCTA GATCCTAGAC |
| | 5701 | AGAGGCTGGG TCAGCTGTGG ATGGGGTGGT GCCTTGGTCT CTCTTGACTA CCTCGTCCAA |
| | 5761 | AGAGAGCACT GCCCTTAGAC AAGAGTTGCT TGTCCTGCTG TGGGCTGGGC TTCCAGCTGC |
| | 5821 | AGACCTCCAG TTGCTTGGTG TTCACTTTGC TCCTCTTGCC CTCTGTCTTC TGGTCCAGGC |
| | 5881 | AGATCAGGGG CTCTGGGGAA ACTGCTGGAA CTCGAGGTGA GGATCAGCCT TTTCCAGCAT |
| | 5941 | CCTGTGAGAG ACCAGAGAGA GAGTTTGGAT TTCATGTGGG GAACCCTCAA GGCCTGTCTG |
| | 6001 | GAGAAGTGAC ACAGGATTTA CTGGGGTGGG CTGGTCCAGG TAGCTCTCCT GAACCTCCTC |
| | 6061 | CTTCCCCAAG CTGAGAAGCT GAGAGCTGGA GGACAATATC CAGGGACATG GCTCTGGAAA |
| | 6121 | ATAACTTTTT TTTTTTTAAG AGACAGGGTC TTGCTCTGTT GTCCAGGCTG GAGGGCAGTG |
| | 6181 | ACATAATCAT AGCTCACTGT ACCCTTGAAC TCCTGGGCTC AAGTGATCCT CCTGCCTCAG |
| | 6241 | CCTCCTTAGT AGCTGGGACT ACCAGTGCAT ACCACCATGC CTGGGTGATT TTTTAAATTT |
| | 6301 | TTTATACAGA CAAGGTCTTG CTATGTTGCC CAGGCTGATC TTGAATTCCC GGGCTCAAGT |
| | 6361 | GGTCCTCCTG CCTCAGCCTC CCACAGGATC GGGATTACAG GCAAGAGCCT CCACGCCCGG |
| | 6421 | CCATGAAATA TAATTCTTAA TATCATACAG GAAAAAGTCA GCGGGTCAAG CTAGCCTGTG |
| | 6481 | GCCCAGCCAC AACTAGCTGA CAAAGCTTCC TGGCCTTCCC TTTAACACAG TTCTGCTGCC |
| | 6541 | ATAGTTCCAT CTATAAAATG GGAATGGAGG GAAATAGGGG AACTGGGAGA GAGAACACAG |
| | 6601 | CCTTGCCAAG CAGCAATGTT AGCCTGATCC TTCCTCCACC TAGCTCGCCA TCTCGCCCTT |
| | 6661 | GGAAAATGGC TCCTGGAGGA TTAGGCAGCC ATCTGCAAGG AGAGGGGCAA CCTGGGACAA |
| | 6721 | GACACCCAGA GGGTAAGGAT TCCAGGAATG AAGCTGCCAT TTCTGGTTGG GAGGAGAAGA |
| | 6781 | GGAAACTTTT AAGAGAAAGG GCTCCATTAT GAGCATGGGT TCAGGGCCCT GCATTACCCA |
| | 6841 | ATCAGAACAG CCGGGATGAG CAGGAGGCCA GCTCCCAGGA GGAAGGGGAA CCCCTTCATA |
| | 6901 | AAGTTCAGAG TGGCTGGGTA GAGTGAGTTG AAGATGCCGG AGGCCGTCAG CATGGCCAGG |
| | 6961 | CTATTCACAC AGGCCACAGC AGAAAAGAGA GCACCTGTGA AGAAATAAAT ACCATACTCT |
| | 7021 | GGAGTCCGAA AGGGCCATAT TCCAACTCTG GCACCACCAC CTCACAGCTG TGTGACCGGG |
| | 7081 | AGTAGTCACT TAACCTATGT CTCCCCTTCC TCACCAGTAA ATCCTGCTAC ATCATGTACT |
| | 7141 | GTGACAAGGA TTCAGTAAGG TCATATGTGG ACAGTAGCTG GCACAGAGGG GCTACTAAAC |
| | 7201 | AAATGGCTGC TATTAAATCC ACATTAAAAG TACATGTGAT CTGACAGAAC CCAGCACATA |
| | 7261 | AAAGAAAAAA AAAGTACATG TGATATTGTC TGATGAAAGC TTGATGGAAA TGGCTTTTTT |
| | 7321 | CTGGTTTATC CTCTTTGGAA TCATCTCCTG TTTGGGATTA ACTGCTGGTC TGATCAGTTC |
| | 7381 | CAATATTCAT AGCGGTGTCA CCACTGAATA GCTTCTTATC CTTTGGGTTC CTGTTCCTCC |
| | 7441 | TTCTGCTAAA TAAGGATAAT ACCTATTTCC TAGATTGTGA GCAACATTAA GTTCACATGG |
| | 7501 | AAATCACCCA TCACTGGGCC TGGTCCCCTG GAAGTAGCTA GTTAGTAAGG GCTGTTCTTT |
| | 7561 | TCTCCTGTTT CTCTTGACAT CTCTGGGCAC AGAGAAAGTG CTGGGAAAAA AAGTTTAGGT |
| | 7621 | GAATGAATGA AGACACATGG ATTCTGGGGA CACCAGAACC CACAGTGGGC TCTGTATGGC |
| | 7681 | ACCAGAGTCT CTGTCATCAT CAGATCCTCA TTCCAGGACA GATGGAAAAA GATGAATGTT |
| | 7741 | TCCAGACTGG GGCATAAAGA CCCAGAGGCT GGAGAAGCTG TTCTTTATAG ATATACCAGG |
| | 7801 | AGAACCCACA GTTTACAAAA TGTGCAACAA CCCAACAGAA GTTGAGATTA AATTCTGTCA |
| | 7861 | CATCTAGAGG GGTCTGTGAT GTCATCAAAA GCAAACCACC CACATCACAG ATGAAGAAAC |
| | 7921 | AGGCCTGTGG CAGGGCTCGG ACTAAAACCC AGATCCTGAG ACCAGCTGCT TTTAAACACA |
| | 7981 | GACGTAGGTT TGCATCCTAG CTCCACCATT TACTGAGTAA CCTTGGGTGA GCCAATGTAA |
| | 8041 | CCCCCTGGGT CTCTGTTTCT TTATCTGTCA ACTGTGGAAA ATGAAACCCA TGTCACAAGG |
| | 8101 | TTGTTCACTT CTGGGCTTGT ACACGCTGAC CCCAGAGAAA CAGGGAACTC TGGCATCACC |
| | 8161 | ACACCCATCT TACAGACGGA AAAGCTGAGG TCTGCAGAGA GTAAATCCTC TGCTCTGGTT |
| | 8221 | ATCTAGAAAG AACATAATTG TGCTCTGCTG ACTGCAAATC CCAACTCTGC GGTTTGAAAA |
| | 8281 | TCCAAGGTGG CATGATCCTC TGCCCATTGT GGGCAATTTC ACAGAAATGT GTTTGTTTTG |
| | 8341 | GCCACTTACT TCTCCAGGGT GAGAGGGGGG AAGGCAAGCT GTTCCCCCAG CCATGGCTGC |
| | 8401 | CCATCAGCCC GTTTCGGGCA GCACTGGACA TGAGGAACCA GACACAGGTG GGTTCTGACA |
| | 8461 | CTCACCCTGC TCTGTCTCTC TCACCAGCTT GGAGAGITTA GCCCGGATGA CAGGTGTGAT |
| | 8521 | GACTAATGAC AGGAAAAGCA ACCCATATCC TGTGGAGAAA CAAACACTCA TCAGGAAAAT |
| | 8581 | GGGGCTGGGG AGAGGGGCGT CCAAGGGAAA GGCAGCAGAG CTCCTATCCA TACCCCACGT |
| | 8641 | GGGGCTTAGG TTAGACCCAG GAAGAACTTC CTTGATGGTG AGGGTGGGAA GACAGTAGTC |
| | 8701 | AAGGAGGAAT GGAGACTGCC CTTGTCTGGG CTTGGCCACC TGCTAGCTCT CATGAATGAA |
| | 8761 | TGCTAATTCC CATTGATTGC TTTCTTGTCT GAACCTCTTG TGGTCACAGC AGGCATCACC |
| | 8821 | CACCCACTTG GCACTTAGTA GGGATATGGC AGGGCACAGA AAACAAGCAT GGGCTTTGGA |
| | 8881 | GTCAGCCCTG AGTTCAAAAC CTGATGCCAT TACATATTAT CTGTGTGGCC TGGGGTACTT |
| | 8941 | ACCCTCTCTG ATCCTGACTC CCTGTATGAG GAAGATAATA AGGCCTTCAT CACAGGATGG |
| | 9001 | TTCTGAGGCA TAGGAGGCTG AATAATGGTG CCCAATGGCA TCAGATTCAT AGCCCTGGAA |
| | 9061 | CCTGTAAATA CTACCTTATT TGGAAAATGA GTCTATGCAG GTGTGCAGTT AAGCCTCCTG |
| | 9121 | AGAGAGCAGA GTTATCCTGG ATTAGGTTGG GCCCTAAATG CCGTCACACA TATCTTTATA |
| | 9181 | AGAGGAAAGC AGACGGAGAT TTGGCACCGA CAGAATTGAG AAGGCACAAA GAGGAGGAGA |
| | 9241 | GTCAATGTGA GCACAGAGGC AGAGACTGGT GATGGCCGCC CCAAGCCAAG GAATGCCAGC |
| | 9301 | AGCCCCAGAA GCTGGAAGAA ATGAGAAACA CGTTCTCTCC TGGAGGCTTG CAAGGGAGCA |
| | 9361 | CTGCCTGCTG ACTGCTTCCA TTCAGCCCGG TGGTACTGAC TTTGGACTTC TGGCCTCCAG |
| | 9421 | AACTGTGAGA GAATATGTTT CTGTTGTGTT AAGCCCCCAA GTTTGTGGTA TGTCATTACA |
| | 9481 | GCAATCTCAG GGAACCAATA CATGAGGTAA AAAGGTAACA TCTATGAAGA GCATGGCATA |
| | 9541 | GGGACACAGC AAATGGGAGT TCCTTTTCCC TTTGCATTCA GTTACTTACA GGCTTCCTGT |
| | 9601 | TTTCTTCATA ACCATTTCTC TCCCTGTGCG ACTGCTGACT CCTCAGCAAA ACTGCAAACT |
| | 9661 | CCTACAGGAC AGTGGATCCT CCAAAGAAGG TATACGATGA GGCATCCAGG GACCCTAGCA |
| | 9721 | GTGTCAGGCC CCTCAAATCC CACTCTGTTG AGACCTCCCC CCGACCCAGA GCAATGACAG |
| | 9781 | CATCTTTATC ATCTCTGCAT CCCCCAGGGC CATCAGCAGG AGGGAAAGGT TCCCTTCTGC |
| | 9841 | TTAATTGTCA GACAAGCAGT TGAGTTAAGA AATCTGTGAT TATTGTATTG TTGACTATAC |
| | 9901 | ACAGCACATT TTAGGGCTCT ATCAAAATAA ATCTGTCCCT TTAAAAAAAG TTAACTAAAG |
| | 9961 | CCGGGCACGG TGGCTCATGC CTGTAATCCC AACACTTTGG GAGGCTGAGG CAGGCGGATC |
| | 10021 | CTTGAGCTCA GGAGTTAGAG ACCTGGACTG GGCAAAATGG TGAGGACCCC ATCTCTATAA |
| | 10081 | AAAATACAAA AATTAGCAAG GTGTGGTAAT GTGCACCAGT GGTCCCAGCT ACTAGAGAGG |
| | 10141 | CCAAGGTGGG AGGATCATCT GGGCCCGGGG GATGAGGCTG CAGTGAGCCA TGATCGTGCC |
| | 10201 | ACTGCACTCT AGCCTGGGTA ACAAAGCGAG ACCCTGTCTC TAAATACATC AATCAAATAA |
| | 10261 | AAATTTTAAA AAGTTAA |
| 65 | 1 | MVLTLLLSAY KLCRFFAMSG PRPGAERLAV PGPDGGGGTG PWWAAGGRGP REVSPGAGTE |
| | 61 | VQDALERALP ELQQALSALK QAGGARAVGA GLAEVFQLVE EAWLLPAVGR EVAQGLCDAI |
| | 121 | RLDGGLDLLL RLLQAPELET RVQAARLLEQ ILVAENRDRV ARIGLGVILN LAKEREPVEL |
| | 181 | ARSVAGILEH MFKHSEETCQ RLVAAGGLDA VLYWCRRTDP ALLRHCALAL GNCALHGGQA |
| | 241 | VQRRMVEKRA AEWLFPLAFS KEDELLRLHA CLAVAVLATN KEVEREVERS GTLALVEPLV |
| | 301 | ASLDPGRFAR CLVDASDTSQ GRGPDDLQRL VPLLDSNRLE AQCIGAFYLC AEAAIKSLQG |
| | 361 | KTKVFSDIGA IQSLKRLVSY STNGTKSALA KRALRLLGEE VPRPILPSVP SWKEAEVQTW |
| | 421 | LQQIGFSKYC ESFREQQVDG DLLLRLTEEE LQTDLGMKSG ITRKRFFREL TELKTFANYS |
| | 481 | TCDRSNLADW LGSLDPRFRQ YTYGLVSCGL DRSLLHRVSE QQLLEDCGIH LGVHRARILT |
| | 541 | AAREMLHSPL PCTGGKPSGD TPDVFISYRR NSGSQLASLL KVHLQLHGFS VFIDVEKLEA |
| | 601 | GKFEDKLIQS VMGARNFVLV LSPGALDKCM QDHDCKDWVH KEIVTALSCG KNIVPIIDGF |
| | 661 | EWPEPQVLPE DMQAVLTFNG IKWSHEYQEA TIEKIIRFLQ GRSSRDSSAG SDTSLEGAAP |
| | 721 | MGPT |

| | |
|---|---|
| 66 | HHHHHHCKRVEQKEECVKLAETEETDKSETMETEDVPTSSRLYWADLKTLLSEKLNSIEFA |
| | DTIKQLSQNTYTPREAGSQKDESLAYYIENQFHEFKFSKVWRDEHYVKIQVKSSIGQNMVTI |
| | VQSNGNLDPVESPEGYVAFSKPTEVSGKLVHANFGTKKDFEELSYSVNGSLVIVRAGEITFA |
| | EKVANAQSFNAIGVLIYMDKNKFPVVEADLALFGHAHLGTGDPYTPGFPSFNHTQFPPSQS |
| | SGLPNIPVQTISRAAAEKLFGKMEGSCPARWNIDSSCKLELSQNQNVKLIVKNVLKERRILNI |
| | FGVIKGYEEPDRYVVVGAQRDALGAGVAAKSSVGTGLLLKLAQVFSDMISKDGFRPSRSIIF |
| | ASWTAGDFGAVGATEWLEGYLSSLHLKAFTYINLDKVVLGTSNFKVSASPLLYTLMGKIM |
| | QDVKHPVDGKSLYRDSNWISKVEKLSFDNAAYPFLAYSGIPAVSFCFCEDADYPYLGTRLD |
| | TYEALTQKVPQLNQMVRTAAEVAGQLIIKLTHDVELNLDYEMYNSKLLSFMKDLNQFKTD |
| | IRDMGLSLQWLYSARGDYFRATSRLTTDFHNAEKTNRFVMREINDRIMKVEYHFLSPYVSP |
| | RESPFRHIFWGSGSHTLSALVENLKLRQKNITAFNETLFRNQLALATWTIQGVANALSGDIW |
| | NIDNEF |
| 67 | HHHHHHCKGVEPKTECERLAGTESPVREEPGEDFPAARRLYWDDLKRKLSEKLDSTDFTG |
| | TIKLLNENSYVPREAGSQKDENLALYVENQFREFKLSKVWRDQHFVKIQVKDSAQNSVIIV |
| | DKNGRLVYLVENPGGYVAYSKAATVTGKLVHANFGTKKDFEDLYTPVNGSIVIVRAGKIT |
| | FAEKVANAESLNAIGVLIYMDQTKFPIVNAELSFFGHAHLGTGDPYTPGFPSFNHTQFPPSRS |
| | SGLPNIPVQTISRAAAEKLFGNMEGDCPSDWKTDSTCRMVTSESKNVKLTVSNVLKEIKILN |
| | IFGVIKGFVEPDHYVVVGAQRDAWGPGAAKSGVGTALLLKLAQMFSDMVLKDGFQPSRSII |
| | FASWSAGDFGSVGATEWLEGYLSSLHLKAFTYINLDKAVLGTSNFKVSASPLLYTLIEKTM |
| | QNVKHPVTGQFLYQDSNWASKVEKLTLDNAAFPFLAYSGIPAVSFCFCEDTDYPYLGTTM |
| | DTYKELIERIPELNKVARAAAEVAGQFVIKLTHDVELNLDYERYNSQLLSFVRDLNQYRADI |
| | KEMGLSLQWLYSARGDFFRATSRLTTDFGNAEKTDRFVMKKLNDRVMRVEYHFLSPYVSP |
| | KESPFRHVFWGSGSHTLPALLENLKLRKQNNGAFNETLFRNQLALATWTIQGAANALSGD |
| | VWDIDNEF |
| 68 | HHHHHHHHGKPIPNPLLGLDSTGGGGSDSAQNSVIIVDKNGRLVYLVENPGGYVAYSKAA |
| | TVTGKLVHANFGTKKDFEDLYTPVNGSIVIVRAGKITFAEKVANAESLNAIGVLIYMDQTKF |
| | PIVNAELSFFGHAHLGGGGGGLPNIPVQTISRAAAEKLFGNMEGDCPSDWKTDSTCRMVTS |
| | ESKNVKLTVS |

1. A SARM1 RNAi agent comprising Formula (I): (R-L)ₙ-P,
wherein R is a double stranded RNA (dsRNA) comprising a sense stand and an antisense strand, wherein the antisense strand is complementary to SARM1 mRNA;
wherein L is a linker, or optionally absent; and
wherein P is a protein comprising one monovalent human TfR binding domain,
wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6; and
wherein n is an integer of 1 to 3.

2. The SARM1 RNAi agent of claim 1, wherein n is 1.

3. The SARM1 RNAi agent of claim 1, wherein n is 2.

4. The SARM1 RNAi agent of claim 1, wherein VH comprises SEQ ID NO: 7 and VL comprises SEQ ID NO: 8.

5. The SARM1 RNAi agent of claim 1, wherein the human TfR binding domain is a Fab, scFv, Fv, or scFab.

6. The SARM1 RNAi agent of claim 1, wherein the human TfR binding domain further comprises a heavy chain constant region comprising cysteine at residue 124 (according to the EU Index numbering).

7. The SARM1 RNAi agent of claim 1, wherein P further comprises a half-life extender.

8. The SARM1 RNAi agent of claim 7, wherein the half-life extender is an immunoglobulin Fc region or a VHH that binds human serum albumin (HSA).

9. The SARM1 RNAi agent of claim 8, wherein the half-life extender is an immunoglobulin Fc region.

10. The SARM1 RNAi agent of claim 9, wherein the immunoglobulin Fc region is a modified human IgG4 Fc region.

11. The SARM1 RNAi agent of claim 10, wherein the modified human IgG4 Fc region comprises proline at residue 228, and alanine at residues 234 and 235 (all residues are numbered according to the EU Index numbering).

12. The SARM1 RNAi agent of claim 9, wherein P comprises an immunoglobulin Fc region comprising cysteine at residue 378 (according to the EU Index numbering).

13. The SARM1 RNAi agent of claim 9, wherein the immunoglobulin Fc region comprises:
(a) a first Fc CH3 domain comprising a serine at position 349, a methionine at position 366, a tyrosine at position 370, and a valine at position 409; and a second Fc CH3 domain comprising a glycine at position 356, an aspartic acid at position 357, a glutamine at position 364, and an alanine at position 407 (all residues are numbered according to the EU Index numbering); or
(b) a first Fc CH3 domain comprising leucine at residue 405, and a second Fc CH3 domain comprising arginine at residue 409 (all residues are numbered according to the EU Index numbering).

14. The SARM1 RNAi agent of claim 1, wherein P comprises one heavy chain (HC) and one light chain (LC), wherein HC comprises SEQ ID NO: 9 and LC comprises SEQ ID NO: 10.

15. The SARM1 RNAi agent of claim 1, wherein P comprises two heavy chains HC1 and HC2 and one light chain LC1, wherein HC1 comprises SEQ ID NO: 14, LC1 comprises SEQ ID NO: 10, HC2 comprises SEQ ID NO: 15.

16. The SARM1 RNAi agent of claim 1, wherein P comprises two heavy chains HC1 and HC2 and one light chain LC1, wherein HC1 comprises SEQ ID NO: 16, LC1 comprises SEQ ID NO: 10, HC2 comprises SEQ ID NO: 17.

17. The SARM1 RNAi agent of claim 8, wherein the half-life extender is a VHH that binds HSA.

18. The SARM1 RNAi agent of claim 17, wherein the VHH comprises CDR1 comprising SEQ ID NO: 20, CDR2 comprising SEQ ID NO: 21, and CDR3 comprising SEQ ID NO: 22.

19. The SARM1 RNAi agent of claim 17, wherein the VHH comprises SEQ ID NO: 23.

20. The SARM1 RNAi agent of claim 17, wherein P comprises one heavy chain (HC) and one light chain (LC), and wherein the HC comprises SEQ ID NO: 11 and the LC comprises SEQ ID NO: 12 or 10.

21. The SARM1 RNAi agent of claim 1, wherein P is a heterodimeric antibody that comprises a first arm comprising one monovalent human TfR binding domain and a second arm that is a null arm.

22. The SARM1 RNAi agent of claim 21, wherein the second arm comprises one heavy chain (HC) and one light chain (LC), and wherein the HC comprises SEQ ID NO: 18 and the LC comprises SEQ ID NO: 19.

23. The SARM1 RNAi agent of claim 21, wherein P comprises two heavy chains HCl and HC2 and two light chains LC1 and LC2, wherein HC1 comprises SEQ ID NO: 13, LC1 comprises SEQ ID NO: 10, HC2 comprises SEQ ID NO: 18, and LC2 comprises SEQ ID NO: 19.

24. The SARM1 RNAi agent of claim 1, wherein L is a Mal-Tet-TCO linker, SMCC linker, GDM linker, MSPT linker or OD linker.

25. The SARM1 RNAi agent of claim 24, wherein L is a SMCC or MSPT linker.

26. The SARM1 RNAi agent of claim 1, wherein P is linked to the 3′ end of the sense strand of dsRNA, optionally via the linker.

27. The SARM1 RNAi agent of claim 1, wherein the sense strand and the antisense strand comprise a pair of nucleic acid sequences selected from the group consisting of:
(a) the sense strand comprises SEQ ID NO: 35, and the antisense strand comprises SEQ ID NO: 36;
(b) the sense strand comprises SEQ ID NO: 37, and the antisense strand comprises SEQ ID NO: 38;
(c) the sense strand comprises SEQ ID NO: 39, and the antisense strand comprises SEQ ID NO: 40; and
(d) the sense strand comprises SEQ ID NO: 41, and the antisense strand comprises SEQ ID NO: 42,
wherein optionally one or more nucleotides of the sense strand and the antisense strand are independently modified nucleotides, and wherein optionally one or more internucleotide linkages of the sense strand and the antisense strand are modified internucleotide linkages.

28. The SARM1 RNAi agent of claim 27, wherein one or more nucleotides of the sense strand are modified nucleotides.

29. The SARM1 RNAi agent of claim 28, wherein each nucleotide of the sense strand is a modified nucleotide.

30. The SARM1 RNAi agent of claim 27, wherein one or more nucleotides of the antisense strand are modified nucleotides.

31. The SARM1 RNAi agent of claim 30, wherein each nucleotide of the antisense strand is a modified nucleotide.

32. The SARM1 RNAi agent of claim 27, wherein the modified nucleotide is a 2′-fluoro modified nucleotide, 2′-O-methyl modified nucleotide, 2′ deoxy nucleotide (DNA), or 2′-O—C16 alkyl modified nucleotide.

33. The SARM1 RNAi agent of claim 27, wherein the sense strand has four 2′-fluoro modified nucleotides at positions 7, 9, 10, and 11 from the 5′ end of the sense strand.

34. The SARM1 RNAi agent of claim 33, wherein nucleotides at positions other than positions 7, 9, 10, and 11 of the sense strand are 2′-O-methyl modified nucleotides.

35. The SARM1 RNAi agent claim 27, wherein the antisense strand has four 2′-fluoro modified nucleotides at positions 2, 6, 14, and 16 from the 5′ end of the antisense strand.

36. The SARM1 RNAi agent of claim 35, wherein nucleotides at positions other than positions 2, 6, 14 and 16 of the antisense strand are 2′-O-methyl modified nucleotides.

37. The SARM1 RNAi agent of claim 27, wherein the sense strand has three 2′-fluoro modified nucleotides at positions 9, 10, and 11 from the 5′ end of the sense strand.

38. The SARM1 RNAi agent of claim 37, wherein nucleotides at positions other than positions 9, 10, and 11 of the sense strand are 2′-O-methyl modified nucleotides.

39. The SARM1 RNAi agent of claim 27, wherein the antisense strand has five 2′-fluoro modified nucleotides at positions 2, 5, 7, 14, and 16 from the 5′ end of the antisense strand.

40. The SARM1 RNAi agent of claim 39, wherein nucleotides at positions other than positions 2, 5, 7, 14, and 16 of the antisense strand are 2′-O-methyl modified nucleotides.

41. The SARM1 RNAi agent of claim 27, wherein the antisense strand has five 2′-fluoro modified nucleotides at positions 2, 5, 8, 14, and 16 from the 5′ end of the antisense strand.

42. The SARM1 RNAi agent of claim 41, wherein nucleotides at positions other than positions 2, 5, 8, 14, and 16 of the antisense strand are 2′-O-methyl modified nucleotides.

43. The SARM1 RNAi agent of claim 27, wherein the antisense strand has five 2′-fluoro modified nucleotides at positions 2, 3, 7, 14, and 16 from the 5′ end of the antisense strand.

44. The SARM1 RNAi agent of claim 43, wherein nucleotides at positions other than positions 2, 3, 7, 14, and 16 of the antisense strand are 2′-O-methyl modified nucleotides.

45. The SARM1 RNAi agent of claim 27, wherein the antisense strand has three 2′-fluoro modified nucleotides at positions 2, 14, and 16 from the 5′ end of the antisense strand.

46. The SARM1 RNAi agent of claim 45, wherein nucleotides at positions other than positions 2, 14, and 16 of the antisense strand are 2′-O-methyl modified nucleotides.

47. The SARM1 RNAi agent of claim 27, wherein the sense strand and the antisense strand have one or more modified internucleotide linkages.

48. The SARM1 RNAi agent of claim 47, wherein the modified internucleotide linkage is phosphorothioate linkage.

49. The SARM1 RNAi agent of claim 48, wherein the sense strand has four or five phosphorothioate linkages.

50. The SARM1 RNAi agent of claim 48, wherein the antisense strand has four or five phosphorothioate linkages.

51. The SARM1 RNAi agent of claim 27, wherein the antisense strand has a phosphate analog at the 5′ end.

52. The SARM1 RNAi agent of claim 51, wherein the phosphate analog is 5′-vinylphosphonate.

53. The SARM1 RNAi agent of claim 27, wherein the sense strand comprises an abasic moiety or inverted abasic moiety.

54. The SARM1 RNAi agent of claim 27, wherein the sense strand and the antisense strand comprise a pair of nucleic acid sequences selected from the group consisting of:
(a) the sense strand comprises SEQ ID NO: 43, and the antisense strand comprises SEQ ID NO: 44, 54, 55, 56, or 60;
(b) the sense strand comprises SEQ ID NO: 45, and the antisense strand comprises SEQ ID NO: 46, 51, 52, 53, or 61;
(c) the sense strand comprises SEQ ID NO: 47, and the antisense strand comprises SEQ ID NO: 48 or 62; and
(d) the sense strand comprises SEQ ID NO: 49, and the antisense strand comprises SEQ ID NO: 50, 57, 58, 59, or 63.

55. The SARM1 RNAi agent of claim 27, wherein the sense strand and the antisense strand have a pair of nucleic acid sequences selected from the group consisting of:
(a) the sense strand consists of SEQ ID NO: 43, and the antisense strand consists of SEQ ID NO: 44, 54, 55, 56, or 60;
(b) the sense strand consists of SEQ ID NO: 45, and the antisense strand consists of SEQ ID NO: 46, 51, 52, 53, or 61;
(c) the sense strand consists of SEQ ID NO: 47, and the antisense strand consists of SEQ ID NO: 48 or 62; and
(d) the sense strand consists of SEQ ID NO: 49, and the antisense strand consists of SEQ ID NO: 50, 57, 58, 59, or 63.

56. A pharmaceutical composition comprising the SARM1 RNAi agent of claim 27 and a pharmaceutically acceptable carrier.

57. A method of reducing axon degeneration in a patient in need thereof, the method comprising administering to the patient an effective amount of the SARM1 RNAi agent of claim 27.

58. A method of treating a SARM1-mediated neurological disease in a patient in need thereof, the method comprising administering to the patient an effective amount of the SARM1 RNAi agent of claim 27.

59. The method of claim 58, wherein the SARM1-mediated neurological disease is selected from amyotrophic lateral sclerosis (ALS, or Lou Gehrig's disease), Alzheimer's disease, Parkinson's disease, multiple sclerosis (MS), Huntington's disease (HD), senile dementia, Pick's disease, Gaucher's disease, Hurler syndrome, progressive multifocal leukoencephalopathy, Alexander's disease, congenital hypomyelination, encephalomyelitis, acute disseminated encephalomyelitis, central pontine myelinolysis, osmotic hyponatremia, Tay-Sachs disease, motor neuron disease, ataxia, spinal muscular atrophy (SMA), Niemann-Pick disease, acute hemorrhagic leukoencephalitis, trigeminal neuralgia, Bell's palsy, cerebral ischemia, multiple system atrophy, Pelizaeus Merzbacher disease, periventricular leukomalacia, a hereditary ataxia, noise-induced hearing loss, congenital hearing loss, age-related hearing loss, Creutzfeldt-Jakob disease, transmissible spongiform encephalopathy, Lewy Body Dementia, frontotemporal dementia, tauopathy, synucleinopathy, amyloidosis, diabetic neuropathy, globoid cell leukodystrophy (Krabbe's disease), Bassen-Komzweig syndrome, transverse myelitis, motor neuron disease, spinocerebellar ataxia, pre-eclampsia, hereditary spastic paraplegias, spastic paraparesis, familial spastic paraplegia, French settlement disease, Strumpell-Lorrain disease, non-alcoholic steatohepatitis (NASH), adrenomyeloneuropathy, progressive supra nuclear palsy (PSP), Friedrich's ataxia, spinal cord injury, acute optic neuropathy (AON), a genetic or idiopathic retinal condition, Leber congenital amaurosis (LCA), Leber hereditary optic neuropathy (LHON), primary open-angle glaucoma (POAG), acute angle-closure glaucoma (AACG), autosomal dominant optic atrophy, retinal ganglion degeneration, retinitis pigmentosa, an outer retinal neuropathy, optic nerve neuritis, optic nerve degeneration associated with multiple sclerosis, Kjer's optic neuropathy, ischemic optic neuropathy, chemotherapy-induced peripheral neuropathy, neuromyelitis optica, Charcot Marie Tooth disease, deficiency in vitamin B12, deficiency in folic acid (vitamin B9), isolated vitamin E deficiency syndrome, non-arteritic anterior ischemic optic neuropathy, exposure to ethambutol, exposure to cyanide, traumatic brain injury (TBI), spinal cord injury, traumatic axonal injury or chronic traumatic encephalopathy (CTE).

60. The method of claim 58, wherein the SARM1 RNAi agent is administered to the patient intravenously or subcutaneously.
